(19) United States
(12) Patent Application Publication
(10) Pub. No.: US 2016/0038519 A1
(43) Pub. Date: Feb. 11, 2016

(54) ADMINISTRATION OF KARENITECIN FOR THE TREATMENT OF ADVANCED OVARIAN CANCER, INCLUDING CHEMOTHERAPY-RESISTANT AND/OR THE MUCINOUS ADENOCARCINOMA SUB-TYPES

(71) Applicant: Frederick H. Hausheer, Fair Oaks Ranch, TX (US)

(72) Inventor: Frederick H. Hausheer, Fair Oaks Ranch, TX (US)

(73) Assignee: BioNumerik Pharmaceuticals, Inc.

(21) Appl. No.: 14/455,847

(22) Filed: Aug. 8, 2014

(51) Int. Cl. A61K 31/695; A61K 31/185; A61K 47/48

(52) U.S. Cl. CPC A61K 31/695; A61K 47/48384; A61K 31/185

(57) ABSTRACT

The present invention discloses and claims methods and compositions for the treatment of platinum and/or taxane cancer treating agent-resistant/-refractory sub-populations and/or the mucinous adenocarcinoma sub-type of ovarian cancer subjects with the silicon-containing highly lipophilic camptothecin derivative (HLCD), Karenitecin (also known as BNP1350; cositecan; 7-[(2′-trimethylsilyl)ethyl]-20(S) camptothecin). The administration of Karenitecin by intravenous (i.v.) and/or oral methodologies are also disclosed and claimed. Karenitecin analogues, including but not limited to, Germanium-substituted Karenitecin, Deuterated Karenitecin, and “flipped” E-ring Karenitecin, are disclosed and claimed. In addition, Karenitecin and one or more cancer treating agents administered either concomitantly or in series via oral and/or i.v. means, are also disclosed and claimed. Methods for the administration of Karenitecin to: (i) increase Progression Free Survival (PFS); (ii) increase the platinum-free time interval; (iii) decrease CA-125 marker levels; and (iv) mitigate or prevent chemotherapeutic drug-resistance from developing are disclosed and claimed herein. Methods for the use of Karenitecin to treat advanced solid tumors; refractory or recurrent solid tumors; recurrent malignant glioma; primary malignant glioma; persistent or recurrent epithelial ovarian or primary peritoneal carcinoma; and other identified cancer types are also disclosed and claimed.

### RELATED APPLICATIONS

The present application claims priority to U.S. Provisional Patent Application Ser. No. 61/865,385, with a filing date of Aug. 13, 2013, and entitled: “ADMINISTRATION OF KARENITECIN FOR THE TREATMENT OF CHEMOTHERAPY-RESISTANT AND/OR THE MUCINOUS SUB-TYPE OF OVARIAN CANCER”, the disclosure of which is herein incorporated by reference in its entirety.

### FIELD OF THE INVENTION

The present invention relates to the use of camptothecin derivatives as anti-cancer drugs. More specifically, the present invention is related to the use of the silicon-containing highly lipophilic camptothecin derivative (HLCD), Karenitecin, for the treatment of advanced ovarian cancer, including platinum/taxane cancer treating agent-resistant sub-populations and/or the mucinous adenocarcinoma-subtype of ovarian cancer.

### BACKGROUND OF THE INVENTION

In brief, the present invention discloses methods for the treatment of platinum and/or taxane cancer treating agent-resistant or -refractory sub-populations and/or the mucinous adenocarcinoma-subtype of ovarian cancer subjects with the silicon-containing highly lipophilic camptothecin derivative (HLCD), Karenitecin (also known as BNP1350; cositecan; 7-[(2′-trimethylsilyl)ethyl]-20(S) camptothecin). The administration of Karenitecin by intravenous (i.v.) or oral methodologies are also disclosed. In addition, Karenitecin analogues, including but not limited to, Germanium-substituted Karenitecin, Deuterated Karenitecin, and “flipped” E-ring Karenitecin, are also disclosed.

### I. Ovarian Cancer

It is estimated that gynecological malignancies account for approximately 18.6% of all new cancer cases diagnosed and approximately 15.3% of all cancer related deaths in women worldwide. Of the gynecological malignancies, ovarian carcinoma is the second most common malignancy after cervical cancer. In 2002, ovarian cancer accounted for 204,200 new cases and 124,700 deaths representing approximately 4.0% of new cancer cases and 4.2% of cancer related deaths in women. See, e.g., Modugno F. Ovarian cancer and polymorphisms in the androgen and progesterone receptor genes. Am. J. Epidemiol. 159(4):319-335 (2004).

In the United States, it is estimated that each year there will be at least approximately 22,400 new cases diagnosed and 15,300 deaths due to ovarian carcinoma, accounting for approximately 3.0% of all cancers in women and causing more deaths than any other cancer of the female reproductive system. See, e.g., American Cancer Society: Cancer Facts and Figures 2009. Atlanta, Ga. American Cancer Society 2009. Unfortunately, as ovarian carcinoma is generally asymptomatic; the majority of subjects are diagnosed with advanced stage disease. Although much research has been conducted over the past several decades, the outcome for subjects with advanced stage ovarian cancer still remains poor, with a 5-year survival rate ranging from less than 10% to 35% for women with stage III or IV disease.

Ovarian cancer is a cancerous growth arising from the ovary. Symptoms are frequently very subtle early on and may include: bloating, pelvic pain, frequent urination, and are easily confused with other illnesses. The three major histologic subtypes of ovarian carcinoma, based on pathologic and clinical features, include epithelial tumors, germ cell tumors, and sex cord-stromal tumors. The majority of ovarian cancers are epithelial in origin, accounting for 80% to 90% of ovarian malignancies. See, e.g., Karlan B Y, Markman M A, Eifel P J. Ovarian cancer, peritoneal carcinoma, and fallopian tube carcinoma. In: DeVita V T Jr, Hellman S, Rosenberg S A, eds. Cancer. Principles & Practice of Oncology. 9th ed. Philadelphia, Pa.: Lippincott Williams & Wilkins; 2011:1368-1391. The epithelial tumors arise from the surface epithelium or serosa of the ovary. In the majority of cases, malignant epithelial ovarian tumors disseminate throughout the peritoneal cavity after exfoliation of malignant cells from the surface of the ovary. Tumor spread also occurs via the lymphatics from the ovary, and spread to lymph nodes is common.

Ovarian cancer is a surgically-staged cancer that is staged using the International Federation of Gynecology and Obstetrics (FIGO) staging system for cancer of the ovary. See, Benedet J L, Pecorelli S, Ngan H Y S, Hacker N F. The FIGO Committee on Gynaecologic Oncology. Staging Classifications and Clinical Practice Guidelines of Gynaecological Cancers. 3rd ed. Elsevier; 2006:95-121. Tumors confined to the ovaries are classified as stage I. A tumor which involves one or both ovaries with pelvic extension is classified as stage II. A tumor which involves one or both ovaries with microscopically-confirmed peritoneal metastases outside the pelvis and/or regional lymph nodes metastasis is classified as stage III. Distant metastasis beyond the peritoneal cavity is classified as stage IV. Liver capsule metastasis is considered stage III, and liver parenchymal metastasis is considered stage IV.

### II. Pharmacology of Platinum Compounds

As previously discussed, the present invention discloses and claims methods for the treatment of platinum and/or taxane cancer treating agent-resistant/-refractory sub-populations and/or the Mucinous sub-type of ovarian cancer subjects.

The anti-neoplastic drug cisplatin (cis-diamminedichloroplatinum or “CDDP”), and related platinum based drugs including carboplatin and oxaliplatin, are widely used in the treatment of a variety of malignancies including, but not limited to, cancers of the ovary, lung, colon, bladder, germ cell tumors and head and neck. Platinum complexes are reported to act, in part, by aquation (i.e., to form reactive aqua species), some of which may predominate intracellularly, and subsequently form DNA intra-strand coordination chelation cross-links with purine bases, thereby cross-linking DNA. The currently accepted paradigm with respect to cisplatin's mechanism of action is that the drug induces its cytotoxic properties by forming a reactive monoaquo species that reacts with the N7 nitrogen contained within the imidazole components of guanine and adenosine found in nuclear DNA to form intrastrand platinum-DNA adducts. However, the exact mechanism of action of cisplatin is not completely understood and remains a subject of research interest within the scientific community. Thus, this mechanism is believed to work predominantly through intra-strand cross-links, and less commonly, through inter-strand cross-links, thereby disrupting the DNA structure and function, which is cytotoxic to cancer cells. Platinum-resistant cancer cells are resilient to the cytotoxic actions of these agents. Certain cancers exhibit intrinsic de novo natural resistance to the killing effects of platinum agents and undergo no apoptosis, necrosis or regression following initial platinum compound treatment. In contrast, other types of cancers exhibit cytotoxic sensitivity to platinum drugs, as evidenced by tumor regression following initial treatment, but subsequently develop an increasing level of platinum resistance, which is manifested as a reduced responsiveness and/or tumor growth following treatment with the platinum drug (i.e., “acquired resistance”). Accordingly, new cancer treating agents are continually being sought which will effectively kill tumor cells, but that are also insensitive or less susceptible to tumor-mediated drug resistance mechanisms that are observed with other platinum agents.

The reaction for cisplatin hydrolysis is illustrated below in Scheme I:

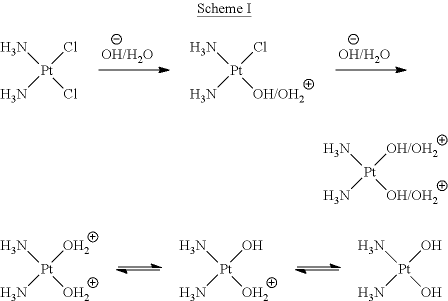

In neutral pH (i.e., pH 7), deionized water, cisplatin hydrolyze to monoaquo/monohydroxy platinum complexes, which is less likely to further hydrolyze to diaqua complexes. However, cisplatin can readily form monoaquo and diaqua complexes by precipitation of chloro ligand with inorganic salts (e.g., silver nitrate, and the like). Also, the chloro ligands can be replaced by existing nucleophile (e.g., nitrogen and sulfur electron donors, etc.) without undergoing aquation intermediates.

Cisplatin is relatively stable in human plasma, where a high concentration of chloride prevents aquation of cisplatin. However, once cisplatin enters a tumor cell, where a much lower concentration of chloride exists, one or both of the chloro ligands of cisplatin is displaced by water to form an aqua-active intermediate form (as shown above), which in turn can react rapidly with DNA purines (i.e., Adenine and Guanine) to form stable platinum-purine-DNA adducts.

Cisplatin enters the cell through both passive diffusion and active transport. The pharmacological behavior of cisplatin is in part determined by hydrolysis reactions that occur once cisplatin is inside the cell where the chloride concentration is essentially zero. In this intracellular milieu, one chlorine ligand is replaced by a water molecule to yield an aquated version of cisplatin. The aquated platinum can then react with a variety of intracellular nucleophiles. Cisplatin binds to RNA more extensively than to DNA and to DNA more extensively than to protein; however, all of these reactions are thought to occur intracellularly. Thus, upon administration, a chloride ligand undergoes slow displacement with water (an aqua ligand) molecules, in a process termed aquation. The aqua ligand in the resulting [PtCl(H₂O)(NH₃)₂]⁺ is easily displaced, allowing cisplatin to coordinate a basic site in DNA. Subsequently, the platinum cross-links two bases via displacement of the other chloride ligand. Cisplatin crosslinks DNA in several different ways, interfering with cell division by mitosis. The damaged DNA elicits various DNA repair mechanisms, which in turn activate apoptosis when repair proves impossible. Most notable among the DNA changes are the 1,2-intrastrand cross-links with purine bases. These include 1,2-intrastrand d(GpG) adducts which form nearly 90% of the adducts and the less common 1,2-intrastrand d(ApG) adducts. 1,3-intrastrand d(GpXpG) adducts may also occur, but are readily excised by the nucleotide excision repair (NER) mechanism. Other adducts include inter-strand crosslinks and nonfunctional adducts that have been postulated to contribute to cisplatin's activity. In some cases, replicative bypass of the platinum 1,2-d(GpG) crosslink can occur allowing the cell to faithfully replicate its DNA in the presence of the platinum cross link, but often if this 1,2-intrastrand d(GpG) crosslink is not repaired, it interferes with DNA replication ultimately resulting in apoptosis.

The formation of cisplatin-DNA adducts that interfere with DNA replication is illustrated in Scheme II:

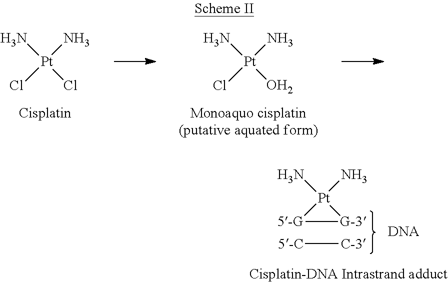

Interaction with cellular proteins, particularly High Mobility Group (HMG) chromosomal domain proteins (which are involved with transcription, replication, recombination, and DNA repair), has also been advanced as a mechanism of interfering with mitosis, although this is probably not its primary method of action. It should also be noted that although cisplatin is frequently designated as an alkylating agent, it has no alkyl group and cannot carry out alkylating reactions. Accordingly, it is more accurately classified as an alkylating-like agent.

### III. Pharmacology of Taxanes

As previously discussed, the present invention discloses and claims methods for the treatment of platinum and/or taxane cancer treating agent-resistant/-refractory sub-populations and/or the Mucinous sub-type of ovarian cancer subjects.

Taxanes are semi-synthetically derived analogues of naturally occurring compounds derived from plants. In particular, taxanes are derived from the needles and twigs of the European yew (Taxus baccata), or the bark of the Pacific yew (Taxus brevifolia). The most widely known taxanes at this time are paclitaxel (Taxol) and docetaxel (Taxotere), which are widely distributed as antineoplastic agents.

Paclitaxel was discovered in the late 1970s, and was found to be an effective antineoplastic agent with a mechanism of action different from then-existing chemotherapeutic agents. Taxanes are recognized as effective agents in the treatment of many solid tumors which are refractory to other antineoplastic agents.

Paclitaxel has the molecular structure shown below as Formula (A):

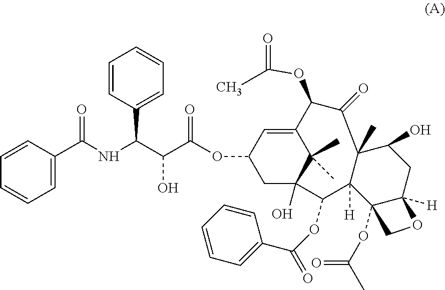

Docetaxel is an analog of Paclitaxel, and has the molecular structure shown below as Formula (B):

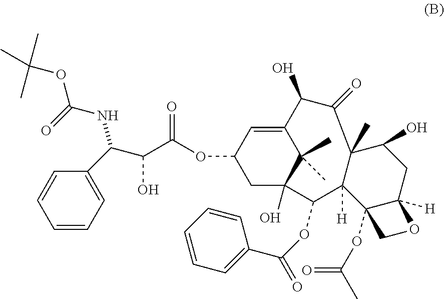

Taxanes exert their biological effects on the cell microtubules and act to promote the polymerization of tubulin, a protein subunit of spindle microtubules. The end result is the inhibition of depolymerization of the microtubules, which causes the formation of stable and nonfunctional microtubules. This disrupts the dynamic equilibrium within the microtubule system, and arrests the cell cycle in the late G₂ and M phases, which inhibits cell replication. Taxanes interfere with the normal function of microtubule growth and arrests the function of microtubules by hyper-stabilizes their structure. This destroys the cell's ability to use its cytoskeleton in a flexible manner.

Taxanes function as an anti-neoplastic agent by binding to the N-terminal 31 amino acid residues of the β-tubulin subunit in tubulin oligomers or polymers, rather than tubulin dimers. Unlike other anti-microtubule agents (e.g., vinca alkaloids) which prevent microtubule assembly, submicromolar concentrations of taxanes function to decrease the lag-time and shift the dynamic equilibrium between tubulin dimers and microtubules (i.e., the hyperpolymerization of tubulin oligomers) toward microtubules assembly and stabilize the newly formed microtubules against depolymerization. The microtubules which are formed are highly stable, thereby inhibiting the dynamic reorganization of the microtubule network. See, e.g., Rowinsky, E. K., et al., Taxol: The prototypic taxane, an important new class of antitumor agents. Semin. Oncol. 19:646 (1992). Tubulin is the “building block” of microtubules, the resulting microtubule/taxane complex does not have the ability to disassemble. Thus, the binding of taxanes inhibit the dynamic reorganization of the microtubule network. This adversely affects cell function because the shortening and lengthening of microtubules (i.e., dynamic instability) is necessary for their function as a mechanism to transport other cellular components. For example, during mitosis, microtubules position the chromosomes during their replication and subsequent separation into the two daughter-cell nuclei.

In addition, even at submicromolar concentrations, the taxanes also induce microtubule bundling in cells, as well as the formation of numerous abnormal mitotic asters (which unlike mitotic asters formed under normal physiological conditions, do not require centrioles for enucleation. Thus, the taxanes function to inhibit the proliferation of cells by inducing a sustained mitotic “block” at the metaphase-anaphase boundary at a much lower concentration than that required to increase microtubule polymer mass and microtubule bundle formation. See, e.g., Rao, S., et al., Direct photoaffinity labeling of tubulin with taxol. J. Natl. Cancer Inst. 84:785 (1992). It should be noted that many of the deleterious side-effects caused by the taxanes are due to the sustained mitotic “block” at the metaphase-anaphase boundary in normal (i.e., non-neoplastic cells).

In addition to stabilizing microtubules, the taxane, paclitaxel, may act as a “molecular sponge” by sequestering free tubulin, thus effectively depleting the cells supply of tubulin monomers and/or dimers. This activity may trigger the aforementioned apoptosis. One common characteristic of most cancer cells is their rapid rate of cell division. In order to accommodate this, the cytoskeleton of the cancer cell undergoes extensive restructuring. Paclitaxel is an effective treatment for aggressive cancers because it adversely affects the process of cell division by preventing this restructuring. Although non-cancerous cells are also adversely affected, the rapid division rate of cancer cells make them far more susceptible to paclitaxel treatment.

Further research has also indicated that paclitaxel induces programmed cell death (apoptosis) in cancer cells by binding to an apoptosis stopping protein called B-cell leukemia 2 (Bcl-2), thus arresting its function.

The molecular structure of taxanes are complex alkaloid esters consisting of a taxane system linked to a four-member oxetan ring at positions C-4 and C-5. The taxane rings of both paclitaxel and docetaxel, but not 10-deacetylbaccatin III, are linked to an ester at the C-13 position. Experimental and clinical studies have demonstrated that analogs lacking the aforementioned linkage have very little activity against mammalian tubulin. Moreover, the moieties at C-2′ and C-3′ are critical with respect to its full biological activity, specifically, for the anti-microtubule hyperpolymerization effect of taxane. The C-2′-OH is of paramount importance for the activity of taxol and the Formula (I) compounds of the present invention, and while the C-2′-OH of taxol can be “substituted” by a sufficiently strong nucleophile (see, PCT/US98/21814; page 62, line 8-27) the biological activity would be greatly diminished. See, e.g., Lataste, H., et al., Relationship between the structures of Taxol and baccatine III derivatives and their in vitro action of the disassembly of mammalian brain. Proc. Natl. Acad. Sci. 81:4090 (1984). For example, it has been demonstrated that the substitution of an acetyl group at the C-2′ position markedly reduces taxane activity. See, e.g., Gueritte-Voegelein, F., et al., Relationships between the structures of taxol analogues and their antimitotic activity. J. Med. Chem. 34:992 (1991).

Taxanes are toxic compounds having a low therapeutic index which have been shown to cause a number of different toxic effects in subjects. The most well-known and severe adverse effects of taxanes are neurotoxicity and hematologic toxicity, particularly anemia and severe neutropenia/thrombocytopenia. Additionally, taxanes also cause hypersensitivity reactions in a large percentage of subjects; gastrointestinal effects (e.g., nausea, diarrhea and vomiting); alopecia; anemia; and various other deleterious physiological effects, even at the recommended dosages. These Taxane medicaments include, in a non-limiting manner, docetaxel or paclitaxel (including the commercially-available paclitaxel derivatives Taxol and Abraxane), polyglutamylated forms of paclitaxel (e.g., Xyotax), liposomal paclitaxel (e.g., Tocosol), and analogs and derivatives thereof.

### SUMMARY OF THE INVENTION

The present invention described and claimed herein has many attributes and embodiments including, but not limited to, those set forth or described or referenced in this Summary section. However, it should be noted that this Summary is not intended to be all-inclusive, nor is the invention described and claimed herein limited to, or by, the features or embodiments identified in said Summary. Moreover, this Summary is included for purposes of illustration only, and not restriction.

The present patent application discloses and claims new and novel inventions which have been derived from the results of a multi-center, multi-national, randomized, open-label, active-controlled, Phase III human clinical study to compare and evaluate the safety and efficacy of the silicon-containing highly lipophilic camptothecin derivative (HLCD) cancer treating drug Karenitecin (also known as BNP1350; cositecan; 7-[(2′-trimethylsilyl)ethyl]-20(S) camptothecin) with that of the camptothecin-analogue chemotherapeutic drug Topotecan; wherein the drugs were administered to the trial subjects as a single, daily intravenous dose of either Karenitecin or Topotecan—[Karenitecin 1.0 mg/m²/day×5 (first 5 consecutive days per cycle) in a 60 minute i.v. infusion or Topotecan 1.5 mg/m²/day×5 (first 5 consecutive days per cycle) in a 30 minute i.v. infusion] every 21 days in subjects with stage III/IV advanced epithelial ovarian cancer who are resistant or refractory to platinum- and taxane-based cancer treating agent regimens, as indicated by relapse/progression while currently on, or within 6 months of completion of, platinum/taxane treatment in a first-line or second-line setting. Such Phase III clinical trial is sometimes referred to herein as the “Karenitecin Phase III Trial”. In addition, subjects with a best response of Stable Disease (“SD”) after a total of 6 cycles of platinum/taxane treatment in the first-line setting were considered platinum-resistant for purposes of the instant Karenitecin Phase III Trial.

In addition, the present patent application discloses and claims new and novel inventions which have been derived from the results of a Phase I clinical trial performed to determine the maximum tolerated dose (MTD) of oral Karenitecin in subjects given in a dose-escalated manner (starting at 0.5 mg/m²) and administered 3-times per week (MWF or TTS) for 3 consecutive weeks followed by a one-week treatment rest. Such Phase I clinical trial of oral Karenitecin is sometimes referred to herein as the “Oral Karenitecin Phase I Trial”.

All subjects admitted to the Karenitecin Phase III Trial were documented to be platinum- and/or taxane-resistant or refractory and have incurable disease. All subjects admitted to the Karenitecin Phase III Trial must have had their disease progress while receiving chemotherapeutic treatment or within 6 months of first or second line platinum- or taxane-based treatment. It is important to note that, currently, there is no FDA-approved chemotherapeutic drug for this specific aforementioned indication.

The Primary Endpoint of the disclosed Karenitecin Phase III Trial was Progression Free Survival (“PFS”); which was defined as the time period from the date of randomization to the date of first radiographically-documented Progressive Disease (“PD”) or date of death due to any cause, taking the event date that occurs first. The date of PD was determined by radiographical objective disease (RECIST) measurement. The Secondary Endpoints of the disclosed Karenitecin Phase III Trial included: (i) Overall Survival (hereinafter “OS”), defined as the time from the date of randomization to the date of death due to any cause; (ii) Incidence of anemia, defined as the proportion of subjects who experience ≧grade 3 anemia based on National Cancer Institute Common Terminology Criteria for Adverse Events (NCI-CTCAE) criteria at any time post-baseline after receiving study treatment; (iii) Incidence of neutropenia (including febrile neutropenia), defined as the proportion of subjects who experience ≧grade 3 neutropenia based on NCI-CTCAE criteria at any time post-baseline after receiving study treatment; and (iv) Incidence of thrombocytopenia, defined as the proportion of subjects who experience ≧grade 3 thrombocytopenia based on NCI-CTCAE criteria at any time post-baseline after receiving study treatment.

Until the results of the Karenitecin Phase III Trial became known, the probability for cure for subjects with advanced ovarian cancer had previously been thought to be remote (with palliation and optimizing the quality of life being the primary treatment goals). The observations in the Karenitecin Phase III Trial regarding increases in the mean and median number of treatment cycles able to be administered to subjects, as well as observations regarding an improved drug administration safety profile, may improve the probability of advanced ovarian cancer being able to be treated as a chronic disease or even for a cure.

In one embodiment of the present invention, a method for the treatment of platinum and/or taxane-refractory or -resistant advanced ovarian cancer, including the mucinous adenocarcinoma-subtype of ovarian cancer, is disclosed, wherein the method is comprised of the oral and/or i.v. administration of Karenitecin in an amount sufficient to provide a therapeutic benefit to the subject having advanced ovarian cancer, including the mucinous adenocarcinoma-subtype of ovarian cancer, which is platinum and/or taxane-refractory or -resistant.

In another embodiment of the present invention, a method for the treatment of a subject having advanced ovarian cancer, including the mucinous adenocarcinoma-subtype of ovarian cancer, and/or where the advanced ovarian cancer is refractory or resistant to platinum and/or taxane cancer treating agents is disclosed, wherein the method is comprised of the oral and/or i.v. administration of Karenitecin in an amount sufficient to provide a therapeutic benefit to the subject having advanced ovarian cancer, including the mucinous adenocarcinoma-subtype of ovarian cancer, and/or where the advanced ovarian cancer is refractory or resistant to platinum and/or taxane cancer treating agents.

In a further embodiment of the present invention, a method for increasing the time period of Progression Free Survival (PFS) in a subject having advanced ovarian cancer, including mucinous adenocarcinoma-subtype of ovarian cancer, is disclosed, wherein the method is comprised of the oral and/or i.v. administration of Karenitecin in an amount sufficient to provide a an increase in the time period of Progression Free Survival (PFS) in the subject having advanced ovarian cancer, including the mucinous adenocarcinoma-subtype of ovarian cancer.

In another embodiment of the present invention, a method for increasing the time period of Progression Free Survival (PFS) in a subject having the mucinous adenocarcinoma-subtype of ovarian cancer and/or where the cancer is refractory or resistant to platinum and/or taxane cancer treating agents is disclosed, wherein the method is comprised of the oral and/or i.v. administration of Karenitecin in an amount sufficient to provide a therapeutic benefit to the subject having the mucinous adenocarcinoma-subtype of ovarian cancer and/or is refractory or resistant to platinum and/or taxane cancer treating agents.

In one embodiment of the present invention, a method for increasing the time period of Progression Free Survival (PFS) while concomitantly reducing cancer treating agent-related toxicities to a subject having advanced ovarian cancer, including the mucinous adenocarcinoma-subtype of ovarian cancer, and/or where the advanced ovarian cancer is refractory or resistant to platinum and/or taxane cancer treating agents is disclosed, wherein the method is comprised of the oral and/or i.v. administration of Karenitecin in an amount sufficient to provide a to provide a an increase in the time period of Progression Free Survival (PFS) while concomitantly reducing various cancer treating agent-related toxicities to the subject having advanced ovarian cancer, including the mucinous adenocarcinoma-subtype of ovarian cancer, and/or where the advanced ovarian cancer is refractory or resistant to platinum and/or taxane cancer treating agents.

The various cancer treating agent-related toxicities include, but are not limited to, hematological, gastrointestinal, anorexia, and other cancer treating agent-related toxicities.

In one embodiment of the present invention, a method for treating a subject having advanced ovarian cancer, including the mucinous adenocarcinoma-subtype of ovarian cancer, and/or where the advanced ovarian cancer is refractory or resistant to platinum and/or taxane cancer treating agents, while also concomitantly reducing the occurrence of cancer treating agent-induced anemia, thrombocytopenia, and/or neutropenia is disclosed, wherein the method is comprised of the oral and/or i.v. administration of Karenitecin in an amount sufficient to provide a therapeutic benefit to the subject having advanced ovarian cancer is disclosed, including the mucinous adenocarcinoma-subtype of ovarian cancer, and/or the advanced ovarian cancer is refractory or resistant to platinum and/or taxane cancer treating agents, while concomitantly reducing the occurrence or grade of occurrence of cancer treating agent-induced anemia, thrombocytopenia, and/or neutropenia.

In one embodiment of the present invention, a method for treating a subject having advanced ovarian cancer while reducing cumulative hematological toxicity to the subject undergoing treatment, including treatment of a subject having the mucinous subtype of ovarian cancer, and/or where the advanced ovarian cancer is refractory or resistant to platinum and/or taxane cancer treating agents is disclosed, wherein the method is comprised of the oral and/or i.v. administration of Karenitecin in an amount sufficient to provide a therapeutic benefit to the subject having advanced ovarian cancer, including the mucinous adenocarcinoma-subtype of ovarian cancer, and/or where the advanced ovarian cancer is refractory or resistant to platinum and/or taxane cancer treating agents.

In another embodiment of the present invention, a method for increasing the total number of cancer treating agent treatment cycles and/or the length of each individual cancer treating cycle in a subject having advanced ovarian cancer, including the mucinous adenocarcinoma-subtype of ovarian cancer, and/or where the advanced ovarian cancer is refractory or resistant to platinum and/or taxane cancer treating agents is disclosed, wherein the method is comprised of the oral and/or i.v. administration of Karenitecin in an amount sufficient to increase the total number of cancer treating agent treatment cycles and/or the length of each individual cancer treating cycle in the subject having advanced ovarian cancer, including the mucinous adenocarcinoma-subtype of ovarian cancer, and/or where the advanced ovarian cancer is refractory or resistant to platinum and/or taxane cancer treating agents.

In one embodiment of the present invention, a method for increasing the platinum-free time interval, the time that elapses after the completion of the initial platinum-based therapy, in a subject having relapsed advanced ovarian cancer, including the mucinous adenocarcinoma-subtype of ovarian cancer, and/or where the advanced ovarian cancer is refractory or resistant to platinum and/or taxane cancer treating agents is disclosed, wherein the method is comprised of the oral and/or i.v. administration of Karenitecin in an amount sufficient to provide a therapeutic benefit to the subject having relapsed advanced ovarian cancer.

In another embodiment of the present invention, a method for increasing the platinum-free time interval, the time that elapses after the completion of the initial platinum-based therapy, in a subject having relapsed advanced ovarian cancer which is refractory or resistant to platinum and/or taxane cancer treating agents is disclosed, wherein the method is comprised of the oral and/or i.v. administration of Karenitecin in an amount sufficient to provide a therapeutic benefit to a subject having relapsed advanced ovarian cancer which is refractory or resistant to platinum and/or taxane cancer treating agents.

In a further embodiment of the present invention, a method for increasing the platinum-free time interval, the time that elapses after the completion of the initial platinum-based therapy, in a subject having a relapse of advanced ovarian cancer, including the mucinous adenocarcinoma-subtype of ovarian cancer, and/or where the cancer is refractory or resistant to platinum and/or taxane cancer treating agents is disclosed, wherein the method is comprised of the oral and/or i.v. administration of Karenitecin in an amount sufficient to provide a therapeutic benefit to the subject having a relapse of advanced ovarian cancer, including the mucinous adenocarcinoma-subtype of ovarian cancer, and/or where the cancer is refractory or resistant to platinum and/or taxane cancer treating agents.

In one embodiment of the present invention, a method to decrease the CA-125 marker levels in a subject having advanced ovarian cancer, including the mucinous adenocarcinoma-subtype of ovarian cancer, and/or where the advanced ovarian cancer is refractory or resistant to platinum and/or taxane cancer treating agents is disclosed, wherein the method is comprised of the oral and/or i.v. administration of Karenitecin in an amount sufficient to decrease the CA-125 marker levels in the subject having advanced ovarian cancer, including the mucinous adenocarcinoma-subtype of ovarian cancer, and/or where the cancer is refractory or resistant to platinum and/or taxane cancer treating agents.

In one embodiment of the present invention, a composition for the treatment of advanced ovarian cancer, including the mucinous adenocarcinoma-subtype of ovarian cancer, and/or where the advanced ovarian cancer is refractory or resistant to platinum and/or taxane cancer treating agents is disclosed, wherein the composition is comprised of Karenitecin and a specific protein-targeting monoclonal antibody (e.g., T-DM1; inotuzumar) attached to or conjugated with the Karenitecin, which is administered (via oral or i.v. means) concomitantly or in series in an amount sufficient to provide a therapeutic benefit to the subject having advanced ovarian cancer, including the mucinous adenocarcinoma-subtype of ovarian cancer, and/or where the cancer is refractory or resistant to platinum and/or taxane cancer treating agents.

In one embodiment of the present invention, a composition for the treatment of advanced ovarian cancer, including the mucinous adenocarcinoma-subtype of ovarian cancer, and/or where the advanced ovarian cancer is refractory or resistant to platinum and/or taxane cancer treating agents is disclosed, wherein the composition is comprised of Karenitecin (administered via oral and/or i.v. means) and one or more cancer treating agents administered concomitantly or in series in an amount sufficient to provide a therapeutic benefit to the subject having advanced ovarian cancer, including the mucinous adenocarcinoma-subtype of ovarian cancer, and/or where the cancer is refractory or resistant to platinum and/or taxane cancer treating agents.

In the aforementioned composition, the cancer treating agents are selected from the group consisting of: fluropyrimidines; pyrimidine nucleosides; purine nucleosides; anti-folates, platinum agents; anthracyclines/anthracenediones; epipodophyllotoxins; camptothecins; vinca alkaloids; taxanes; epothilones; antimicrotubule agents; alkylating agents; antimetabolites; topoisomerase inhibitors; aziridine-containing compounds; antivirals; hormones; hormonal complexes; antihormonals; enzymes, proteins, peptides and polyclonal and/or monoclonal antibodies and various other cytotoxic and cytostatic agents; as well as (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, or

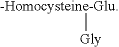

Various other embodiments of the present invention, methods allowing various treatment schedules (e.g., M, W, and F; on a daily basis, etc.) and reductions in hematologic- and non-hematologic-based toxicities in a subject having advanced ovarian cancer, including the mucinous adenocarcinoma-subtype of ovarian cancer, and/or where the cancer is refractory or resistant to platinum and/or taxane cancer treating agents is disclosed, wherein the method is comprised of the oral and/or i.v. administration of Karenitecin in an amount sufficient to provide reduced hematologic- and non-hematologic-based toxicities in the subject having advanced ovarian cancer, including the mucinous adenocarcinoma-subtype of ovarian cancer, and/or where the cancer is refractory or resistant to platinum and/or taxane cancer treating agents.

In a further embodiment of the present invention, a method which may be curative in a subject having advanced ovarian cancer, including the mucinous adenocarcinoma-subtype of ovarian cancer, and/or where the cancer is refractory or resistant to platinum and/or taxane cancer treating agents, due to the ability of the method to circumvent cancer treating agent drug-resistance in said cancer, is disclosed, wherein the method is comprised of the oral and/or i.v. administration of Karenitecin in an amount sufficient to circumvent cancer treating agent drug-resistance in the subject having advanced ovarian cancer, including the mucinous adenocarcinoma-subtype of ovarian cancer, and/or where the cancer is refractory or resistant to platinum and/or taxane cancer treating agents.

In yet another embodiment of the present invention, a composition for the treatment of a subject having advanced ovarian cancer, including the mucinous adenocarcinoma-subtype of ovarian cancer, and/or where the advanced ovarian cancer is refractory or resistant to platinum and/or taxane cancer treating agents is disclosed, wherein the composition is comprised of Karenitecin, Germanium-substituted Karenitecin, deuterated Karenitecin, and/or “flipped” E-ring Karenitecin administered by oral and/or i.v. methodologies in an amount sufficient to provide a therapeutic benefit to the subject having advanced ovarian cancer, including the mucinous adenocarcinoma-subtype of ovarian cancer, and/or where the advanced ovarian cancer is refractory or resistant to platinum and/or taxane cancer treating agents.

In one embodiment of the present invention, a method for treating a subject having advanced ovarian cancer, including the mucinous adenocarcinoma-subtype of ovarian cancer, and/or where the advanced ovarian cancer is refractory or resistant to platinum and/or taxane cancer treating agents, while also concomitantly reducing the occurrence or grade of occurrence of cancer treating agent-induced toxicities and/or improving the side-effect profile of cancer treating agent administration is disclosed, wherein the method is comprised of the oral and/or i.v. administration of one or more camptothecin cancer treating agents in an amount sufficient to provide a therapeutic benefit to the subject having advanced ovarian cancer, including the mucinous adenocarcinoma-subtype of ovarian cancer, and/or the advanced ovarian cancer is refractory or resistant to platinum and/or taxane cancer treating agents, while concomitantly reducing the occurrence or grade of occurrence of cancer treating agent-induced toxicities.

In another embodiment, a method for the treatment of a subject having one or more cancer types selected from the group consisting of: (i) advanced solid tumors; (ii) refractory or recurrent solid tumors; (iii) recurrent malignant glioma; (iv) primary malignant glioma; (v) third-line treatment of persistent or recurrent epithelial ovarian or primary peritoneal carcinoma; (vi) malignant melanoma; and/or (vii) relapsed or refractory non-small cell lung cancer is disclosed; wherein the method is comprised of the oral and/or i.v. administration of Karenitecin in an amount sufficient to provide a therapeutic benefit to the subject having one or more cancer types selected from the group consisting of: (i) advanced solid tumors; (ii) refractory or recurrent solid tumors; (iii) recurrent malignant glioma; (iv) primary malignant glioma; (v) third-line treatment of persistent or recurrent epithelial ovarian or primary peritoneal carcinoma; (vi) malignant melanoma; and/or (vii) relapsed or refractory non-small cell lung cancer.

In an embodiment, a method to increase the progression-free survival (PFS) in a subject diagnosed with the mucinous adenocarcinoma subtype of ovarian cancer is disclosed.

In a further embodiment, a method to induce an increase in the progression-free survival (PFS) of a subject who is either refractory or resistant to platinum- and/or taxane-based cancer treating agents and/or had the mucinous adenocarcinoma sub-type of ovarian cancer is disclosed.

In one embodiment, a method to induce a reduction of grade 3 or 4 anemia events, a reduction of grade 3 or 4 thrombocytopenia events, and a reduction of grade 4 neutropenia events in a subject during treatment with Karenitecin is disclosed.

In a further embodiment, a method for increasing the total number of cancer treating agent treatment cycles and/or the length of each individual cancer treating agent cycle capable of being tolerated by a subject having advanced cancer and/or where the subject's cancer is refractory or resistant to one or more cancer treating agents is disclosed, and wherein the cancer is further selected from the group consisting of: colorectal cancer, gastric cancer, esophageal cancer, cancer of the biliary tract, gallbladder cancer, breast cancer, brain cancer and cancer of the Central Nervous System, cervical cancer, ovarian cancer, endometrial cancer, vaginal cancer, uterine cancer, prostate cancer, hepatic cancer, adenocarcinoma, pancreatic cancer, lung cancer, myeloma, lymphoma, and cancers of the blood.

In another embodiment, a method to adjust the timing and dosage of Karenitecin administered to a subject having advanced ovarian cancer, including the mucinous adenocarcinoma-subtype of ovarian cancer, and/or where the subject's advanced ovarian cancer is resistant or refractory to platinum and/or taxane cancer treating agents is disclosed; wherein the adjustment of the timing and dosage of Karenitecin administration is based upon cancer antigen 125 (CA-125) marker levels in said subject having advanced ovarian cancer, including the mucinous adenocarcinoma-subtype of ovarian cancer, with the CA-125 marker levels being measured: (i) prior to beginning the treatment regimen with Karenitecin, and (ii) during the treatment regimen with Karenitecin, with both the time interval between CA-125 marker level measurements and the amount of Karenitecin administered to said subject being dependent upon the CA-125 marker levels which measured in said subject having advanced ovarian cancer, including the mucinous adenocarcinoma-subtype of ovarian cancer. By way of non-limiting example, a patient receiving treatment with Karenitecin in the Karenitecin Phase III Trial discussed herein was observed to have a reduction in CA-125 marker levels from a high of 2072 U/ML to a low of 167 U/ML. Another patient receiving treatment with Karenitecin in the Karenitecin Phase III Trial discussed herein was observed to have a reduction in CA-125 marker levels from a high of 237 U/ML to a low of 24 U/ML.

In another embodiment, the adjustment of the timing and dosage of Karenitecin administered is based upon the levels of the Mucin 16 marker (MUC16) levels in a subject having advanced ovarian cancer, including the mucinous adenocarcinoma-subtype of ovarian cancer, and/or where the subject's advanced ovarian cancer is refractory or resistant to platinum and/or taxane cancer treating agents is disclosed.

In yet another embodiment, a method to treat cancers histologically-characterized as being of the mucinous type; wherein said method is comprised of the i.v. and/or oral administration of Karenitecin in an amount sufficient to provide a therapeutic benefit to the subject having one or more cancers which have been histologically-characterized as being of the mucinous type; and where the cancer is further selected from the group consisting of: colorectal cancer, gastric cancer, esophageal cancer, cancer of the biliary tract, gallbladder cancer, breast cancer, brain cancer and cancer of the Central Nervous System, cervical cancer, ovarian cancer, endometrial cancer, vaginal cancer, uterine cancer, prostate cancer, hepatic cancer, adenocarcinoma, pancreatic cancer, lung cancer, myeloma, lymphoma, and cancers of the blood.

In another embodiment, a method to treat a subject having advanced ovarian cancer, including the mucinous adenocarcinoma-subtype of ovarian cancer, and/or where the subject's advanced ovarian cancer is refractory or resistant to platinum and/or taxane cancer treating agents, and where said subject is also suffering from cancer treating agent-associated toxicity or toxicities is disclosed.

In a further embodiment, a method for increasing Progression Free Survival (PFS) in a subject with advanced ovarian cancer; where the subject's advance ovarian cancer has been differentiated into detailed ovarian cancer histological sub-categories selected from the group consisting of: (i) the “Histological Stage: G1-well differentiated” sub-category; (ii) the ECOG Performance Status 2″ sub-category; (iii) the ECOG Performance Status 0″ sub-category; (iv) the Histopathology class: Adenocarcinoma (grade ≧2) not otherwise specified sub-category; (v) the Histopathology class: serous adenocarcinoma” sub-category; (vi) the Histological Stage: G2-moderately differentiated” sub-category; (vii) the FIGO Stage IV″ sub-category; (viii) the FIGO Stage IV″ sub-category; (ix) the FIGO Stage IIIB; “Ovary as primary site of disease” sub-category; and (x) the best response stable disease (SD) after 6 cycles in a first-line setting sub-category; and where the method is comprised of the oral and/or i.v. administration of a therapeutically-effective dose of Karenitecin to the subject with advanced ovarian cancer; wherein said subject's advance ovarian cancer has been differentiated into detailed ovarian cancer histological sub-categories is disclosed.

Another embodiment discloses a method for increasing Overall Survival (OS) in a subject with advanced ovarian cancer; where the subject's advance ovarian cancer has been differentiated into detailed ovarian cancer histological sub-categories selected from the group consisting of: (i) the mucinous adenocarcinoma ovarian cancer sub-category; (ii) the advanced epithelial ovarian cancer sub-category (based upon observed increase in average survival time); (iii) the FIGO Stage IV″ sub-category; (iv) the Histopathology class: Adenocarcinoma (grade ≧2) not otherwise specified; (iv) the Histological Stage: G1-well differentiated” sub-category; and (v) the Histopathology Class: Undifferentiated carcinoma” sub-category; and where the method is comprised of the oral and/or i.v. administration of a therapeutically-effective dose of Karenitecin to the subject with advanced ovarian cancer; wherein said subject's advance ovarian cancer has been differentiated into detailed ovarian cancer histological sub-categories.

In another embodiment, a method to reduce or prevent cancer treating agent-induced toxicities in a subject having advanced ovarian cancer, including the mucinous adenocarcinoma-subtype of ovarian cancer, and/or where the subject's advanced ovarian cancer is refractory or resistant to platinum and/or taxane cancer treating agents, and where said subject is also suffering from cancer treating agent-associated toxicity or toxicities; where the method is comprised of the oral and/or i.v. administration of a therapeutically-effective dose of Karenitecin to the subject having advanced ovarian cancer, including the mucinous adenocarcinoma-subtype of ovarian cancer, and/or where the subject's advanced ovarian cancer is refractory or resistant to platinum and/or taxane cancer treating agents, and where the subject is also suffering from cancer treating agent-associated toxicity or toxicities is disclosed.

In yet another embodiment, a method to improve the Quality of Life (QOL) in a subject having advanced ovarian cancer, including the mucinous adenocarcinoma-subtype of ovarian cancer, and/or where the subject's advanced ovarian cancer is refractory or resistant to platinum and/or taxane cancer treating agents, and where said subject is also suffering from cancer treating agent-associated toxicity or toxicities; where the said method is comprised of the oral and/or i.v. administration of a therapeutically-effective dose of Karenitecin to the subject having advanced ovarian cancer, including the mucinous adenocarcinoma-subtype of ovarian cancer, and/or where the subject's advanced ovarian cancer is refractory or resistant to platinum and/or taxane cancer treating agents, and where the subject is also suffering from cancer treating agent-associated toxicity or toxicities is disclosed.

### BRIEF DESCRIPTION OF THE FIGURES

FIG. 1: illustrates, in bar graph form, the relative five (5) year survival of subjects with Stage I to Stage IV invasive epithelial ovarian cancer.

FIG. 2: Summary of experiments evaluating effect of BNP7787 on BNP1350-induced cytotoxicity—JHOM2B data.

FIG. 3: Summary of experiments evaluating effect of BNP7787 on BNP1350-induced cytotoxicity—OMC3 data.

FIG. 4: Summary of experiments evaluating effect of BNP7787 on BNP1350-induced cytotoxicity—COV644 data.

### DETAILED DESCRIPTION OF THE INVENTION

The descriptions and embodiments set forth herein are not intended to be exhaustive, nor do they limit the present invention to the precise forms disclosed. They are included to illustrate the principles of the invention, and its application and practical use by those skilled in the art.

### Listing of Terms Utilized in Present Patent Application

Included is a listing of some of the terms used herein. However, it should be noted that this listing of terms and the definitions set forth herein are provided solely as guidance for the reader. Unless otherwise explained, all technical and scientific terms used herein have the same meaning as commonly understood by one of ordinary skill in the art to which this disclosure belongs. Although methods and materials similar or equivalent to those described herein can be used in the practice of the disclosed methods and compositions, suitable methods and materials are described below. All publications, patent applications, patents, and other references mentioned herein are incorporated by reference in their entirety. In case of conflict, the present Specification, including explanations of terms, will control. In addition, the materials, methods, and examples are for illustrative purposes only, and are not intended to be limiting.

As utilized herein, the term “adenocarcinoma” refers to a cancer that originates in glandular tissue. Glandular tissue comprises organs that synthesize a substance for release such as hormones. Glands can be divided into two general groups: (i) endocrine glands—glands that secrete their product directly onto a surface rather than through a duct, often into the blood stream and (ii) exocrine glands—glands that secrete their products via a duct, often into cavities inside the body or its outer surface. However, it should be noted that to be classified as adenocarcinoma, the tissues or cells do not necessarily need to be part of a gland, as long as they have secretory properties. Adenocarcinoma may be derived from various tissues including, but not limited to, breast, colon, lung, prostate, salivary gland, stomach, liver, gall bladder, pancreas (99% of pancreatic cancers are ductal adenocarcinomas), ovary, cervix, vagina, and uterus, as well as unknown primary adenocarcinomas. Adenocarcinoma is a neoplasm which frequently presents marked difficulty in differentiating from where and from which type of glandular tissue the tumor(s) arose. Thus, an adenocarcinoma identified in the lung may have had its origins (or may have metastasized) from an ovarian adenocarcinoma. Cancer for which a primary site cannot be found is called cancer of unknown primary.

As utilized herein, the term “adjuvant therapy” means additional treatment of a subject with cancer given after the primary treatment or surgery to lower the risk that the cancer will come back. Adjuvant therapy may include treatment with cancer treating agents such as cancer treating agents, radiation therapy, hormones, cytotoxic or cytostatic agents, antibodies, and/or Karenitecin.

As utilized herein, the medical definitions for the terms “adverse effect”, “adverse event”, “adverse experience”, “adverse reaction”, and “unexpected adverse reaction” have previously been agreed to by consensus of the more than thirty Collaborating Centers of the WHO International Drug Monitoring Centre (Uppsala, Sweden). See, Edwards, I. R., et al., Harmonisation in Pharmacovigilance Drug Safety 10(2):93-102 (1994). The following medical definitions, with input from the WHO Collaborative Centre, have been agreed to:

1. Adverse Event (Adverse Effect or Adverse Experience)—Any untoward medical occurrence in a patient or clinical investigation subject administered a pharmaceutical product and which does not necessarily have to have a causal relationship with this treatment. An Adverse Event (AE) can therefore be any unfavorable and unintended sign (including an abnormal laboratory finding, for example), symptom, or disease temporally associated with the use of a medicinal product, whether or not considered related to the medicinal product.

2. Adverse Drug Reaction (ADR)—In the pre-approval clinical experience with a new medicinal product or its new usages, particularly as the therapeutic dose(s) may not be established: all noxious and unintended responses to a medicinal product related to any dose should be considered adverse drug reactions. Drug-related Adverse Events are rated from grade 1 to grade 5 and relate to the severity or intensity of the event. Grade 1 is mild, grade 2 is moderate, grade 3 is severe, grade 4 is life threatening, and grade 5 results in death.

3. Unexpected Adverse Drug Reaction—An adverse reaction, the nature or severity of which is not consistent with the applicable product information.

### Serious Adverse Event or Adverse Drug Reaction:

A Serious Adverse Event (experience or reaction) is any untoward medical occurrence that at any dose:

(a) Results in death or is life-threatening. It should be noted that the term “life-threatening” in the definition of “serious” refers to an event in which the patient was at risk of death at the time of the event; it does not refer to an event which hypothetically might have caused death if it were more severe.
(b) Requires inpatient hospitalization or prolongation of existing hospitalization.
(c) Results in persistent or significant disability/incapacity, or
(d) Is a congenital anomaly/birth defect.

As utilized herein the term “cancer” refers to all known forms of cancer including, solid forms of histopathologically classified forms of cancer (e.g., those that form tumors), lymphomas, and non-solid tumors e.g., leukemias.

As used herein, the phrase “an amount sufficient to provide a therapeutic benefit” or “a therapeutically-effective” amount” in reference to the medicaments, compounds, or compositions of the instant invention refers to the administered dosage that is sufficient to induce a desired biological, pharmacological, or therapeutic outcome(s) in a subject suffering from one or more types of cellular metabolic anomalies or other pathophysiological conditions, including cancer. By way of non-limiting example and with regard to cancer, such outcome(s) can include: (i) cure or remission of previously observed cancer(s); (ii) shrinkage of tumor size; (iii) reduction in the number of tumors; (iv) delay or prevention in the growth or reappearance of cancer; (v) selectively sensitizing cancer cells to the activity of the anti-cancer agents; (vi) restoring or increasing apoptotic effects or sensitivity in tumor cells; and/or (vii) increasing the time of survival of the subject, alone or while concurrently experiencing reduction, prevention, mitigation, delay, shortening the time to resolution of, alleviation of the signs or symptoms of the incidence or occurrence of an expected side-effect(s), toxicity, disorder or condition, or any other untoward alteration in the subject.

As utilized herein the term “cancer” refers to all known forms of cancer including, solid forms of cancer (e.g., tumors), lymphomas, and leukemias.

As used herein, the term “cancer treating agent”, “cancer treating agents”, “cancer treatment agent” or “chemotherapeutic agent” refer to medicament(s) that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. The cancer treating agents of the present invention include, but are not limited to: (i) chemotherapeutic agents (e.g., fluropyrimidines, pyrimidine nucleosides, purine nucleosides, anti-folates, platinum agents, anthracyclines/anthracenediones, epipodophyllotoxins, camptothecins, vinca alkaloids, taxanes, epothilones, antimicrotubule agents, alkylating agents, antimetabolites, topoisomerase inhibitors, and the like); (ii) hormones, hormonal complexes, and antihormonals (e.g., interleukins, interferons, leuprolide, pegasparaginase, and the like); (iii) enzymes, proteins, and peptides; antivirals (e.g., acyclovir, zidovudine, and the like); (iv) cytotoxic agents and cytostatic agents; (v) polyclonal and monoclonal antibodies, including agents selected from the group consisting of crizotinib, gefitinib, erlotinib, cetuximab, afatinib, dacomitinib, ramucirumab, necitumumab, lenvatinib, palbociclib, alectinib, zybrestat, tecemotide, obinutuzumab (GA101), AZD9291, CO-1686, vintafolide, CRLX101, ipilimumab, yervoy, nivolumab, ibrutinib, selumetinib, olaparib, trastuzumab, lucitanib, rucaparib, NOV-002, MPDL3280A, pembrolizumamb, lambrolizumab (MK-3475), MEDI4736, tremelimumab, AMP-514, MEDI6469, RG7446, CRS-207, GVAX, ceritinib (LDK378), IMCgp100, vemurafenib (Zelboraf), cabozantinib, CTL019, LEE011, T-DM1, MM-121, bavituximab, MAGE-A3, axitinib, ipilimumab, protein-targeted monoclonal antibodies, rituximab, tivantinib, and the like; (vi) PD-1 checkpoint receptor inhibiting agents, PD-L1 checkpoint receptor inhibiting agents, and other checkpoint receptor inhibiting agents; (vii) immune checkpoint pathway modulatory antibodies; (viii) kinase inhibitors; (ix) ALK inhibitors; (x) cancer vaccines; (xi) Antibody Drug Conjugates; (xii) chemoenhancing agents; and (xiii) chimeric antigen receptor T-cell (CAR-T) Therapy.

As utilized herein, the terms “cancer treating agent regimen(s)” or “cancer treating agent therapy” or “chemotherapy treatment cycle”, or “treatment cycle” or “cancer treating agent cycle”, or cancer treating agent treatment cycle” refer to treatment using one or more of the cancer treating agents, mentioned above, with or without the use of the sulfur-containing small molecules of the present invention.

As used herein, the terms “cancer treatment agent(s)” or “cancer treatment drug(s)” or “cancer treatment composition(s)” or “chemotherapeutic agent(s)” refer to a medicament or medicaments that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. The cancer treating agents of the present invention include, but are not limited to: (i) chemotherapeutic agents (e.g., fluropyrimidines, pyrimidine nucleosides, purine nucleosides, anti-folates, platinum agents, anthracyclines/anthracenediones, epipodophyllotoxins, camptothecins, vinca alkaloids, taxanes, epothilones, antimicrotubule agents, alkylating agents, antimetabolites, topoisomerase inhibitors, and the like); (ii) hormones, hormonal complexes, and antihormonals (e.g., interleukins, interferons, leuprolide, pegasparaginase, and the like); (iii) enzymes, proteins, and peptides; antivirals (e.g., acyclovir, zidovudine, and the like); (iv) cytotoxic agents and cytostatic agents; (v) polyclonal and monoclonal antibodies, including agents selected from the group consisting of crizotinib, gefitinib, erlotinib, cetuximab, afatinib, dacomitinib, ramucirumab, necitumumab, lenvatinib, palbociclib, alectinib, zybrestat, tecemotide, obinutuzumab (GA101), AZD9291, CO-1686, vintafolide, CRLX101, ipilimumab, yervoy, nivolumab, ibrutinib, selumetinib, olaparib, trastuzumab, lucitanib, rucaparib, NOV-002, MPDL3280A, pembrolizumamb, lambrolizumab (MK-3475), MEDI4736, tremelimumab, AMP-514, MEDI6469, RG7446, CRS-207, GVAX, ceritinib (LDK378), IMCgp100, vemurafenib (Zelboraf), cabozantinib, CTL019, LEE011, T-DM1, MM-121, bavituximab, MAGE-A3, axitinib, ipilimumab, protein-targeted monoclonal antibodies, rituximab, tivantinib, and the like; (vi) PD-1 checkpoint receptor inhibiting agents, PD-L1 checkpoint receptor inhibiting agents, and other checkpoint receptor inhibiting agents; (vii) immune checkpoint pathway modulatory antibodies; (viii) kinase inhibitors; (ix) ALK inhibitors; (x) cancer vaccines; (xi) Antibody Drug Conjugates; (xii) chemoenhancing agents; and (xiii) chimeric antigen receptor T-cell (CAR-T) Therapy.

Cancer treatment agents of the present invention include, but are not limited to: (i) chemotherapeutic agents (e.g., fluropyrimidines, pyrimidine nucleosides, purine nucleosides, anti-folates, platinum agents, anthracyclines/anthracenediones, epipodophyllotoxins, camptothecins, vinca alkaloids, taxanes, epothilones, antimicrotubule agents, alkylating agents, antimetabolites, topoisomerase inhibitors, and the like); (ii) hormones, hormonal complexes, and antihormonals (e.g., interleukins, interferons, leuprolide, pegasparaginase, and the like); (iii) enzymes, proteins, and peptides; antivirals (e.g., acyclovir, zidovudine, and the like); (iv) cytotoxic agents, cytostatic agents; (v) polyclonal and monoclonal antibodies (e.g., crizotinib, gefitinib, erlotinib, cetuximab, afatinib, dacomitinib, ramucirumab, necitumumab, lenvatinib, palbociclib, alectinib, zybrestat, tecemotide, obinutuzumab (GA101), AZD9291, CO-1686, vintafolide, CRLX101, ipilimumab, yervoy, nivolumab, ibrutinib, selumetinib, olaparib, trastuzumab, lucitanib, rucaparib, NOV-002, MPDL3280A, pembrolizumamb, lambrolizumab (MK-3475), MEDI4736, tremelimumab, AMP-514, MEDI6469, RG7446, CRS-207, GVAX, ceritinib (LDK378), IMCgp100, vemurafenib (Zelboraf), cabozantinib, CTL019, LEE011, T-DM1, MM-121, bavituximab, MAGE-A3, axitinib, ipilimumab, rituximab, tivantinib, and the like); (vi) PD-1, PD-L1, and other checkpoint receptor inhibiting agents; (vii) immune checkpoint pathway modulatory antibodies; (viii) kinase inhibitors; (ix) ALK inhibitors; (x) cancer vaccines; (xi) Antibody Drug Conjugates; and (xii) chimeric antigen receptor T-cell (CAR-T) Therapy.

As utilized herein, the terms “cancer treating agent effect” or “chemotherapeutic effect” or “cytotoxic or cytostatic activities” refer to the ability of an agent/medicament/composition to reduce, prevent, mitigate, limit, and/or delay the growth of metastases or neoplasms, or kill neoplastic cells directly by necrosis or apoptosis of neoplasms or any other mechanism, or that can be otherwise used to reduce, prevent, mitigate, limit, and/or delay the growth of metastases or neoplasms in a subject with neoplastic disease.

As utilized herein, the terms “cancer treating agent cycle”, or “cancer treating agent regimen(s)” refer to treatment using the above-mentioned cancer treating agents with or without the compounds of the present invention.

As utilized herein, the term “cancer treatment agent effect” refers to the ability of an agent/medicament/composition to reduce, prevent, mitigate, limit, and/or delay the growth of metastases or neoplasms, or kill neoplastic cells directly by necrosis or apoptosis of neoplasms or any other mechanism, or that can be otherwise used to reduce, prevent, mitigate, limit, and/or delay the growth of metastases or neoplasms in a subject with neoplastic disease.

As utilized herein, the term “chemoenhancing agent(s)” refers to agents that enhance or improve the cancer treating ability or performance of cancer treating agents. The term chemoenhancing agents includes: (i) 2,2′-dithio-bis-ethane sulfonate, including disodium 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly,

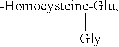

-Homocysteine-Glu-Gly, Homocysteine-Glu, and pharmaceutically-acceptable salts thereof.

As utilized herein, the term “cumulative treatment-related toxicity” refers to cumulative and/or irreversible toxicities that frequently occur with repeated cycles of cancer treating agents. Recognition of recurrent ovarian cancer as a disease with significant secondary responses and remissions has led to an increase in the need for oncologists to plan for the long-term therapy of patients. However, many of the currently available front-line and salvage agents used in advanced ovarian cancer are associated with cumulative and/or irreversible toxicities that pose challenges in such long-term planning. The irreversible effects associated with some of these therapies may render patients less tolerant to subsequent treatments and lead to a cycle of diminishing treatment options with each remission and disease relapse. Additionally, the potential for patients to experience cumulative toxicity must be carefully weighed against the goals of prolonging the disease-free interval and improving patient quality of life. A number of agents are available in the treatment armamentarium (e.g., platinum, paclitaxel, gemcitabine, etoposide, liposomal doxorubicin, and topotecan), many, but not all, of which are associated with cumulative toxicity. Although therapeutic regimens that couple platinum-based therapy with other cytotoxic agents are the current standard of care for advanced ovarian cancer patients, the cumulative toxicities of cisplatin and carboplatin can present barriers to the long-term use of these agents. Further, the severe myelogenic toxicity and greater incidences of secondary myelodysplasia and leukemia associated with prolonged and cumulative etoposide treatment must also be considered. Cumulative doxorubicin and paclitaxel exposure must also be monitored to minimize the risk of patient morbidity due to cardiotoxicity and neuropathy, respectively. Gemcitabine shares many overlapping toxicities with other agents, and care must be taken during combination regimens to avoid synergy of these effects. The main toxicity associated with topotecan is noncumulative, manageable myelosuppression.

As utilized herein, the terms “cycle” “cancer treating agent cycle” or “cancer treatment cycle” or “chemotherapeutic cycle” refers to the administration of a complete regimen of medicaments to the patient in need thereof in a defined time period.

As used herein, the term “cytostatic agents” are mechanism-based agents that slow the progression of neoplastic disease and include drugs, biological agents, and radiation. As used herein the term “cytotoxic agents” are any agents or processes that kill neoplastic cells and include drugs, biological agents, immunotherapy; and radiation. In addition, the term “cytotoxic” is inclusive of the term “cytostatic”.

As used herein, “a therapeutically-effective amount”, “therapeutically-effective dose” or “a pharmaceutically-effective amount” in reference to the compounds or compositions of the instant invention refers to the amount that is sufficient to induce a desired biological, pharmacological, or therapeutic outcome in a subject with neoplastic disease. That result can be reduction, prevention, mitigation, delay, shortening the time to resolution of, alleviation of the signs or symptoms of, or exert a medically-beneficial effect upon the underlying pathophysiology or pathogenesis of an expected or observed side-effect, toxicity, disorder or condition, or any other desired alteration of a biological system. In the present invention, the result will generally include or allow the reduction, prevention, mitigation, delay in the onset of, and/or attenuation of the severity of cancer treating agent-associated toxicity; an increase in the frequency and/or number of treatments; an increase in duration of cancer treating agent therapy; an increase or improvement in Progression Free Survival (PFS); an increase or improvement in Overall Survival (OS); and/or Complete Remission (CR).

As utilized herein, the terms “Hazard Ratio”, “HR”, and “hazard ratio” refer to the chance of an event occurring with treatment “A” divided by the chance of the event occurring with treatment “B”. The hazard ratio is an expression of the hazard or chance of events occurring in one treatment arm as a ratio of the hazard of the events occurring in the other treatment arm. A hazard ratio less than 1.0 means that treatment “A” is more favorable than treatment “B” in terms of the result being measured. As described herein for purposes of data references to hazard ratios, treatment “A” refers to treatment with Karenitecin and treatment “B” refers to treatment with Topotecan. Accordingly, a hazard ratio less than 1.0 relating to Karenitecin treatment refers to a more favorable outcome in the result being measured for Karenitecin treatment in comparison to the result being measured for the treatment other than Karenitecin. References to an “improvement” or “reduction” in the hazard ratio in favor of Karenitecin refer to a more favorable outcome in the result being measured for Karenitecin treatment in comparison to the result being measured for the treatment other than Karenitecin.

As used herein, the term “Highly Lipophilic Camptothecin Derivatives (HLCD) is defined as camptothecin analogs having a water solubility of less than 5 μg/mL of water.

As utilized herein, the term “maintenance therapy” means the ongoing or chronic use of an agent to help lower the risk of recurrence (return of cancer) after it has disappeared or been substantially reduced or diminished or not detectable following initial therapy or surgery. Maintenance therapy also may be used for patients with advanced cancer (cancer that cannot be cured) to help keep it from growing and spreading farther.

As used herein, the term “mg/m²” represents the amount of a given compound or formulation in milligrams per square meter of the total body surface area of the subject to whom the compound or formulation is administered.

As utilized herein, the term “mucinous tumors” are part of the surface epithelial-stromal tumor group of ovarian neoplasms, and account for approximately 36% of all ovarian tumors. Approximately 75% are benign, 10% are borderline, and 15% are malignant. Rarely, the tumor is seen bilaterally, with only approximately 5% of primary mucinous tumors being bilateral in nature. Benign mucinous tumors are typically multilocular (have several lobes), and the cysts have a smooth lining of epithelium that resembles endocervical epithelial cells with small numbers of gastrointestinal-type epithelial cells. Borderline and malignant mucinous tumors often have papillae and solid areas. There may also be hemorrhage and necrosis. It is well documented that malignancy may be only focally present in mucinous neoplasms of the ovary, so thorough sampling of the entire tumor is imperative. The major distinguishing features of mucinous tumors are that the tumors are filled with a mucus-like material, which gives them their name; this mucus is produced by mucus-secreting goblet cells, very similar to the cells lining normal intestine. In addition, mucinous tumors may become very large, with some weighing as much as 25 kilograms.

As utilized herein, the term “neoadjuvant therapy” means treatment given as a first step to shrink a tumor before the main treatment or surgery is conducted. Neoadjuvant therapy may include treatment with cancer treating agents such as chemotherapeutic agents, radiation therapy, hormones, cytotoxic or cytostatic agents, antibodies and/or Karenitecin. Neoadjuvant therapy is intended to make later treatment or surgery easier and more likely to succeed, and reduce the consequences of a more extensive treatment or surgical technique that would be required if the tumor wasn't reduced in size or extent.

As used herein, the terms “platinum cancer treating agents” or “platinum medicaments” or “platinum compounds” include all compounds, compositions, and formulations which contain a platinum ligand in the structure of the molecule. By way of non-limiting example, the valence of the platinum ligand contained therein may be platinum II or platinum IV. The platinum medicaments or platinum compounds disclosed in the present invention include, in a non-limiting manner, cisplatin, oxaliplatin, carboplatin, satraplatin, and analogs and derivatives thereof.

The term “platinum-free interval” is defined herein as the time that elapses after the completion of the initial platinum-based therapy (including combination therapies involving the administration of platinum-based agents) in a subject having a relapse of ovarian cancer before further treatment of the subject with a platinum- and/or taxane-based chemotherapeutic agent occurs.

The term “platinum sensitivity” is defined herein as the disease-free or treatment-free interval in a subject having ovarian cancer, after the treatment of the subject with a platinum-based cancer treatment agent, including combination therapy involving a platinum-based agent. Platinum sensitivity has emerged as an important and significant predictive indicator of response to second-line cancer treatment agent regimen.

As utilized herein, the term “protein-targeted monoclonal antibody” refers to a new class of monoclonal antibodies that can effectively reach inside a cancer cell, a key goal for these important anticancer agents, as most proteins that cause or are associated with cancer are located intracellularly. Scientists from Memorial Sloan Kettering Cancer Center and Eureka Therapeutics have collaborated to develop the new monoclonal antibody, ESK1, that targets the intracellular, oncogenic WT1 protein. WT1, which is overexpressed in a range of leukemias and other cancers, including myeloma, breast, ovarian, and colorectal cancers. ESK1 was engineered to mimic the functions of a T cell receptor, a key component of the immune system. T cells have a receptor system that is designed to recognize proteins that are inside the cell. As intracellular proteins are degraded as part of normal cellular processes, HLA molecules carry fragments of those proteins to the cell surface. When T cells recognize certain peptides as abnormal, the T cell kills the diseased cell.

As used herein the term “Quality of Life” or “QOL” refers, in a non-limiting manner, to a maintenance or increase in a cancer subject's overall physical and mental state (e.g., cognitive ability, ability to communicate and interact with others, decreased dependence upon analgesics for pain control, maintenance of ambulatory ability, maintenance of appetite and body weight (lack of cachexia), lack of or diminished feeling of “hopelessness”; continued interest in playing a role in their treatment, and other similar mental and physical states). An improvement in Quality of Life includes, without limitation, improvements due to: the ease of administration of a cancer treating agent, an increase in the number of cancer treating agent treatment cycles able to be tolerated by the subject; and/or the reduced impact of the toxicity or toxic effects of a cancer treating agent.

As used herein, the term “reducing” includes preventing and/or attenuating the overall severity of, delaying the initial onset of, and/or expediting the resolution of the acute and/or chronic pathophysiology associated with malignancy in a subject.

As used herein, the term “refractory”, refers to a subject who is suffering from an ovarian cancer which fails to respond reasonably in a favorable manner in terms of tumor shrinkage or duration of stabilization or shrinkage in response to treatment with a platinum and/or taxane cancer treatment agent(s) in the first line setting. Such patients have a best response of stable disease or their tumor(s) progress during such treatment and have a poor prognosis.

As used herein, the term “resistant”, with respect to a platinum- and/or taxane-based cancer treatment agent, refers to a subject who is suffering from an ovarian cancer which fails to respond to treatment with a platinum and/or taxane cancer treatment agent(s) for a time of greater than 6 months or more. By way of non-limiting example, subjects with a best response of Stable Disease (“SD”) after a total of 6 cycles of platinum and/or taxane treatment in the first-line setting were considered to be platinum-resistant for purposes of the Karenitecin Phase III clinical trial disclosed in the present patent application.

As used herein, the term “taxane cancer treating agents” or “taxane medicaments” or taxane chemotherapy agents” “include, in a non-limiting manner, docetaxel or paclitaxel (including the commercially-available paclitaxel derivatives Taxol® and Abraxane®), polyglutamylated forms of paclitaxel (e.g., Xyotax®), liposomal paclitaxel (e.g., Tocosol®), and analogs and derivatives thereof.

During the past 30 years, the camptothecins (CPTs), of which Karenitecin (also known as BNP1350; cositecan; 7-[(2′-trimethylsilyl)ethyl]-20(S) camptothecin) is a member, have emerged as an important new class of antitumor drugs. Currently, two water-soluble CPT derivatives have been approved by the United States Food and Drug Administration (FDA) for use in the treatment of subjects with cancer. The first, Camptosar (irinotecan HCl, Pfizer, Inc; hereinafter referred to as “CPT-11”), is a water-soluble CPT analog that is indicated as a component of first-line therapy in combination with 5-fluorouracil and leucovorin for subjects with metastatic carcinoma of the colon or rectum, and is also indicated for subjects with metastatic carcinoma of the colon or rectum whose disease has recurred or progressed following initial fluorouracil-based therapy. See, Camptosar [package insert]. New York, N.Y.: Pfizer, Inc. (2006).

The second, Hycamtin (Topotecan HCl, GlaxoSmithKline; hereinafter referred to as “Topotecan”), is a water-soluble CPT analogue that is indicated for the treatment of metastatic carcinoma of the ovary after failure of initial or subsequent cancer treating agent regimen(s); small cell lung cancer sensitive disease after failure of first-line cancer treating agent therapy; and stage IV-B, recurrent, or persistent carcinoma of the cervix which is not amenable to curative treatment with surgery and/or radiation therapy. See, Hycamtin [package insert]. Research Triangle Park, NC: GlaxoSmithKline (2006).

The objective of the Karenitecin Phase III Trial, as disclosed in the present patent application, was to determine the safety and efficacy of intravenous Karenitecin versus Topotecan in subjects with platinum- and/or taxane-resistant or refractory advanced ovarian cancer.

### I. Ovarian Cancer

It has been estimated that gynecological malignancies account for approximately 18.6% of all new cancer cases diagnosed and approximately 15.3% of all cancer related deaths in women worldwide. Of the gynecological malignancies, ovarian carcinoma is the second most common malignancy after cervical cancer. In 2002, ovarian cancer accounted for 204,200 new cases and 124,700 deaths representing approximately 4.0% of new cancer cases and 4.2% of cancer related deaths in women. See, e.g., Modugno F. Ovarian cancer and polymorphisms in the androgen and progesterone receptor genes. Am. J. Epidemiol. 159(4):319-335 (2004).

In the United States, it is estimated that each year there will be at least approximately 22,400 new cases diagnosed and 15,300 deaths due to ovarian carcinoma, accounting for approximately 3.0% of all cancers in women and causing more deaths than any other cancer of the female reproductive system. See, e.g., American Cancer Society: Cancer Facts and Figures 2009. Atlanta, Ga. American Cancer Society 2009. The lifelong risk of developing sporadic epithelial ovarian cancer is approximately 1.7%, although subjects with a familial predisposition have a much higher lifetime risk, in the range of 10% to 40%. See, e.g., Jemal, A. et al., Cancer Statistics 2009. CA Cancer J. Clin. 59:225 (2009). The median age of diagnosis for sporadic disease is 60 years old, although subjects with a genetic predisposition may develop this type of tumor earlier, often in their fifth decade. The age-specific incidence of sporadic disease increases with age from 15-16 per 100,000 in the 40- to 44-year old age group, to a peak rate of 57 per 100,000 in the 70- to 74-year old age group. See, Id.

Unfortunately, as ovarian carcinoma is generally asymptomatic; the majority of subjects are diagnosed with advanced stage disease. Although much research has been conducted over the past several decades, the outcome for subjects with advanced stage ovarian cancer still remains poor, with a 5-year survival rate ranging from less than 10% to 35% for women with stage III or IV disease.

Ovarian cancer is a cancerous growth arising from the ovary. Symptoms are frequently very subtle early on and may include: bloating, pelvic pain, frequent urination, and are easily confused with other illnesses. The three major histologic subtypes of ovarian carcinoma, based on pathologic and clinical features, include epithelial tumors, germ cell tumors, and sex cord-stromal tumors. The majority of ovarian cancers are epithelial in origin, accounting for 80% to 90% of ovarian malignancies. See, e.g., Karlan B Y, Markman M A, Eifel P J. Ovarian cancer, peritoneal carcinoma, and fallopian tube carcinoma. In: DeVita V T Jr, Hellman S, Rosenberg S A, eds. Cancer. Principles & Practice of Oncology. 9th ed. Philadelphia, Pa.: Lippincott Williams & Wilkins; 2011:1368-1391. The epithelial tumors arise from the surface epithelium or serosa of the ovary. In the majority of cases, malignant epithelial ovarian tumors disseminate throughout the peritoneal cavity after exfoliation of malignant cells from the surface of the ovary. Tumor spread also occurs via the lymphatics from the ovary, and spread to lymph nodes is common.

Ovarian cancer is a surgically-staged cancer using the International Federation of Gynecology and Obstetrics (FIGO) staging system for cancer of the ovary and uses information obtained after surgery, which can include a total abdominal hysterectomy, removal of (usually) both ovaries and fallopian tubes, removal of (usually) the omentum, and pelvic (peritoneal) washing to assess any cytopathology therein. See, Benedet J L, Pecorelli S, Ngan H Y S, Hacker N F. The FIGO Committee on Gynecologic Oncology. Staging Classifications and Clinical Practice Guidelines of Gynaecological Cancers. 3rd ed. Elsevier; 2006:95-121. The ovaries contain the ova and secrete the hormones that control the reproductive cycle. Removing the ovaries and the fallopian tubes greatly reduces the amount of the hormones estrogen and progesterone circulating in the body. This can halt or slow breast and ovarian cancers that need these hormones to grow.

The general FIGO stages for ovarian cancer are set forth below:
Stage I—limited to one or both ovaries
   IA—involves one ovary; capsule intact; no tumor on ovarian surface; no malignant cells in ascites or peritoneal washings
   IB—involves both ovaries; capsule intact; no tumor on ovarian surface; negative washings
   IC—tumor limited to ovaries with any of the following: capsule ruptured, tumor on ovarian surface, positive washings
Stage II—pelvic extension or implants
   IIA—extension or implants onto uterus or fallopian tube; negative washings
   IIB—extension or implants onto other pelvic structures; negative washings
   IIC—pelvic extension or implants with positive peritoneal washings
Stage III—peritoneal implants outside of the pelvis; or limited to the pelvis with extension to the small bowel or omentum
   IIIA—microscopic peritoneal metastases beyond pelvis
   IIIB—macroscopic peritoneal metastases beyond pelvis <2 cm in size
   IIIC—peritoneal metastases beyond pelvis >2 cm or lymph node metastases
Stage IV—distant metastases to the liver or outside the peritoneal cavity

FIGO histopathologic classification of epithelial ovarian neoplasms includes: (i) serous tumors; (ii) mucinous tumors; (iii) endometrioid tumors; (iv) clear cell tumors; (v) Brenner tumors; (vi) undifferentiated tumors (of epithelial structure, but are poorly-differentiated); and (vii) mixed epithelial tumors. Epithelial ovarian tumors are then further sub-classified by grading: (a) Gx—grade cannot be assessed; (b) G1—well differentiated; (c) G2—moderately differentiated; and (d) G3—poorly differentiated.

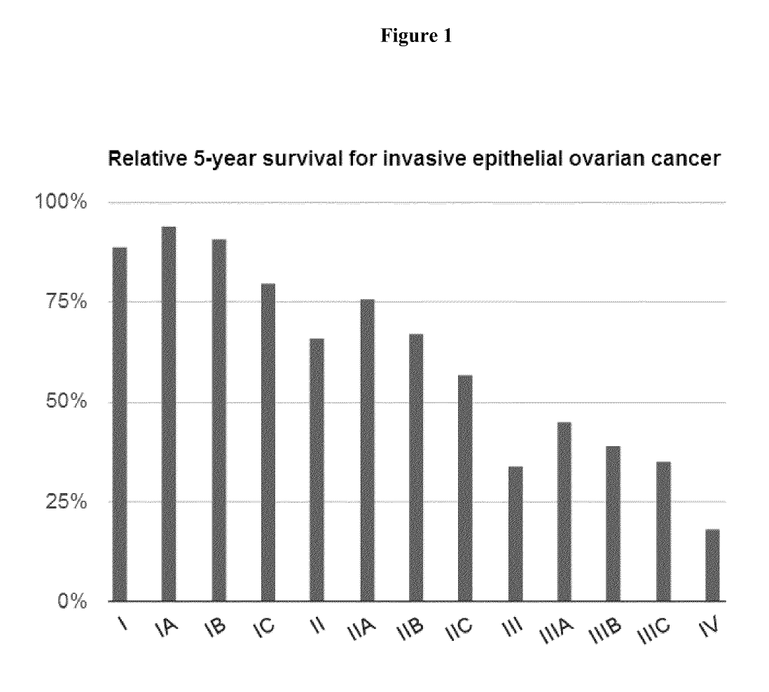

Generally, the prognoses of all ovarian tumors are independently affected by the following: (i) the specific stage of the cancer at time of diagnosis; (ii) the histological subtype and grading; and (iii) the volume of residual disease. Other important prognostic factors include: (a) performance status; (b) the platinum-free interval; (c) response of CA-125 levels to initial treatment; and (d) progression of the disease. See, e.g., Benedet J L, Pecorelli S, Ngan H Y S, Hacker N F. The FIGO Committee on Gynecologic Oncology. Staging Classifications and Clinical Practice Guidelines of Gynaecological Cancers. 3rd ed. Elsevier; 2006:95-121. Epithelial carcinoma of the ovary is often described as a “silent killer” because the majority of subjects do not present with symptoms until the disease has spread outside the ovary and pelvis (approximately 70% of subjects with epithelial cancers of the ovary present with stage III or IV disease). See, e.g., Karlan B Y, Markman M A, Eifel P J. Ovarian cancer, peritoneal carcinoma, and fallopian tube carcinoma. In: DeVita V T Jr, Hellman S, Rosenberg S A, eds. Cancer. Principles & Practice of Oncology. 9th ed. Philadelphia, Pa.: Lippincott Williams & Wilkins; 2011:1368-1391. Patients with stage III disease have a 5-year survival rate of approximately 35%, which is dependent on the volume of disease in the upper abdomen. Patients with stage IV disease have a 5-year survival rate of less than 10%. After the administration of a post-operative platinum-based combination cancer treating agent therapy (including combination therapy with paclitaxel), 4-year survival rates for subjects with optimal stage III disease (defined as only microscopic residual disease) is approximately 60%. See, Id. The relative five (5) year survival of subjects with Stage I to Stage V invasive epithelial ovarian cancer is illustrated in FIG. 1.

In most cases, the exact cause of ovarian cancer remains unknown. Epithelial ovarian cancer is a clonal disease that arises from a single cell in more than 90% of cases. Multiple genetic changes must occur in the ovarian surface epithelium (OSE) to produce malignant transformation. Repeated rupture and repair (ovulation) of the OSE provides this opportunity for genetic aberrations. Hereditary factors are implicated in approximately 5-13% of all ovarian cancers. Thus far, the syndromes that have been identified are: (i) the Breast-Ovarian Cancer Syndrome, linked to an inherited mutation in the BRCA1 and the BRCA2 genes (this includes site specific Ovarian Cancer Syndrome); and (ii) Type II Lynch Syndrome, which also includes colon, breast, endometrial and prostate cancer in affected individuals. See, e.g., Lynch H T, Watson P, Lynch J F, Conway T A, Fili M. Hereditary ovarian cancer. Heterogeneity in age at onset. Cancer 71(2 Suppl): 573-81 (1993); Struewing J P, Hartge P, Wacholder S, et al. The risk of cancer associated with specific mutations of BRCA1 and BRCA2 among Ashkenazi Jews. N. Engl. J. Med. 336:1401-1408 (1997).

As discussed above, the BRCA1 and BRCA2 genes, encoding breast cancer susceptibility protein, type 1 and 2, respectively, account for 5-13% of ovarian cancers and certain populations (e.g., Ashkenazi Jewish women) are at a higher risk of both breast cancer and ovarian cancer, often at an earlier age than the general population. See, e.g., Lakhani S R, Manek S, Penault-Llorca F., et al., Pathology of ovarian cancer in BRCA1 and BRCA2 carriers. Clin. Cancer Res. 10(7):2473-2481 (2004).

In the United States, 10-20% of women with ovarian cancer have a first- or second-degree relative with either breast or ovarian cancer. Mutations in either of two major susceptibility genes, BRCA1 and BRCA2, confer a lifetime risk of breast cancer of between 60-85% and a lifetime risk of ovarian cancer of between 15-40%. However, mutations in these genes account for only 2-3% of all breast cancers. See, e.g., Wooster R, Weber B L. Breast and Ovarian Cancer N. Engl. J. Med. 348(23):2339-2347 (2003).

A strong family history of uterine cancer, colon cancer, or other gastrointestinal cancers may indicate the presence of a syndrome known as hereditary nonpolyposis colorectal cancer (HNPCC, also known as Type II Lynch syndrome), which confers a higher risk for developing ovarian cancer. Patients with strong genetic risk for ovarian cancer may consider the use of prophylactic oophorectomy (i.e., the surgical removal of both ovaries) after completion of childbearing years. Prophylactic oophorectomy significantly reduces the chances of developing both breast cancer and ovarian cancer. Women with BRCA gene mutations generally also have their fallopian tubes removed at the same time (salpingo-oophorectomy), since they also have an increased risk of fallopian tube cancer.

Hereditary breast-ovarian cancer syndrome (HBOC) produce higher than normal levels of both breast cancer and ovarian cancer in genetically related families (either one individual suffered from both, or several individuals in the families suffered from one or the other disease). The hereditary factors may be proven or suspected to cause the pattern of breast and ovarian cancer occurrences in the family.

Several other molecular abnormalities have been identified in subjects with epithelial ovarian cancer, although their contribution to malignant transformation is poorly understood. These abnormalities include: (i) deletions of 3p, 6q, 8p, and 10q; (ii) loss of heterozygosity is commonly observed on 11p, 13q, 16q, 17p, and 17q; (iii) mutations of the p53 oncogene occurs in over 50% of subjects; (iv) amplification of HER2/neu gene is found in approximately 8% of subjects and confers of poorer prognosis; and (v) expression of angiogenic cytokines such as vascular endothelial growth factor (VEGF) is frequently observed and confers a worse prognosis. See, e.g., Karlan B Y, Markman M A, Eifel P J. Ovarian cancer, peritoneal carcinoma, and fallopian tube carcinoma. In: DeVita V T Jr, Hellman S, Rosenberg S A, eds. Cancer. Principles & Practice of Oncology. 9th ed. Philadelphia, Pa.: Lippincott Williams & Wilkins; 2011:1368-1391.

The increased risk of developing ovarian cancer appears to be affected by several factors, including, but not limited to: (i) older women, and in those who have a first or second degree relative with the disease; (ii) hereditary forms of ovarian cancer can be caused by mutations in specific genes (most notably BRCA1, BRCA2, and genes for hereditary nonpolyposis colorectal cancer); (iii) infertile women; (iv) women with endometriosis; and (v) women who have used or currently use postmenopausal estrogen replacement therapy.

Combination oral contraceptive pills have been shown to provide a protective factor for ovarian cancer. See, e.g., Bandera, C A. Advances in the understanding of risk factors for ovarian cancer. J Reprod Med 50(6):399-406 (2005). The relationship between use of oral contraceptives and ovarian cancer was shown in a summary of results of 45 case-control and prospective studies. Cumulatively these studies show a protective effect for ovarian cancers. Women who used oral contraceptives for 10 years had about a 60% reduction in risk of ovarian cancer. (a risk ratio 0.42 with statistically significant confidence intervals given the large study size). This was, by far, the largest epidemiological study to date on the subject (45 studies, over 20,000 women with ovarian cancer and about 80,000 controls). See, e.g., Collaborative Group on Epidemiological Studies of Ovarian Cancer, Beral V, Doll R, Hermon C, Peto R, Reeves G. Ovarian cancer and oral contraceptives: collaborative reanalysis of data from 45 epidemiological studies including 23,257 women with ovarian cancer and 87,303 controls. Lancet 371:(9609):303-314 (2008).

The link to the use of fertility medications (e.g., Clomiphene citrate) has been controversial. An analysis in 1991 raised the possibility that use of drugs may increase the risk of ovarian cancer. However, several cohort studies and case-control studies have been subsequently conducted without demonstrating conclusive evidence for such a link. Thus, it will remain a complex topic to study as the infertile population differs in parity from the “normal” population. See, Id.

Early age at first pregnancy, older age of final pregnancy, and the use of low dose hormonal contraception have also been shown to have a protective effect. The risk is also lower in women who have had their fallopian tubes blocked surgically (tubal ligation). See, e.g., Bandera C A. Advances in the understanding of risk factors for ovarian cancer. J. Reprod. Med. 50(6): 399-406 (2005). Tentative evidence suggests that breastfeeding lowers the risk developing ovarian cancer. See, e.g., Hunn, J; Rodriguez, G C. Ovarian cancer: etiology, risk factors, and epidemiology. Clin. Obstet. Gynecol. 55(1): 3-23 (2012).

### II. Mucinous Adenocarcinoma Sub-Type of Ovarian Cancer

Mucinous tumors, in general, are part of the surface epithelial-stromal tumor group of ovarian neoplasms. Mucinous adenocarcinomas account for approximately 36% of all ovarian tumors. See, e.g., Smith J A and Wolf J K. Ovarian Cancer. In: Pharmacotherapy: A pathophysiologic approach, 8th ed. Dipiro J T, Talbert R L, Yee G C, et al., —editors, New York: McGraw-Hill 2361-2375 (2008). Approximately 75% of mucinous tumors are benign, 10% are borderline, and 15% are malignant. Rarely, the tumor is seen bilaterally, with only approximately 5% of primary mucinous tumors being bilateral in nature.

Benign mucinous tumors are typically multilocular (have several lobes), and the cysts have a smooth lining of epithelium that resembles endocervical epithelial cells with small numbers of gastrointestinal-type epithelial cells. Borderline and malignant mucinous tumors often have papillae and solid areas. There may also be hemorrhage and necrosis. It is well documented that malignancy may be only focally present in mucinous neoplasms of the ovary, so thorough sampling of the entire tumor is imperative.

The major distinguishing features of mucinous tumors are that the tumors are filled with a mucus-like material, which gives them their name; this mucus is produced by mucus-secreting goblet cells, very similar to the cells lining normal intestine. In addition, mucinous tumors may become very large, with some weighing as much as 25 kilograms.

Cystadenocarcinomas (malignant tumors) contain a more solid growth pattern with the hallmarks of malignancy: (i) cellular atypia and stratification, (ii) loss of the normal architecture of the tissue, and (iii) necrosis. The appearance can look similar to colonic cancer. Clear stromal invasion is used to differentiate borderline tumors from malignant tumors. Pseudomyxoma peritonei may present as a result of an ovarian mucinous tumor, however this is a rare cause of this condition, which is a rare condition. A more common cause of Pseudomyxoma peritonei is a mucin-producing tumor of the appendix.

Since mucinous tumors arising from the ovary usually only involve one ovary, the presence of involvement in both ovaries with a mucinous tumor suggests that the tumor may have arisen in another location, and further study is warranted. The risk of mucinous tumors is significantly associated with smoking: relative risk for current smokers 2.22 (2.22 times the risk for non-smokers) and 2.02 for past smokers. Risk is also associated with smoking duration: relative risk per 20 years was 1.44. See article by Tworoger S S in Cancer Mar. 1, 2008 using data from the Nurses Health Study.

As previously stated, mucinous adenocarcinomas comprise approximately 36% of ovarian epithelial neoplasms. Mucinous carcinomas generally occur in women in the fourth to seventh decades. The mean age is 54 years. The clinical presentation is similar to that of benign mucinous neoplasms, inasmuch as the majority of cases are Stage I. Rather, as these tumors can be quite large, the clinical presentation is generally that of a large pelvic or abdominal mass and abdominal distention. Recurrence and mortality in Stage I mucinous carcinoma occurs in 8-12% of cases, similar to well-staged Stage I serous carcinomas. Advanced stage mucinous carcinoma (FIGO III and IV) is currently viewed as uniformly fatal, as is serous carcinoma of advanced stage. Limited data are available on FIGO Stage II tumors, as only 10% of subjects are in Stage II. Similarly, little data are available on high grade Stage I mucinous carcinomas. Occult advanced stage disease is rare compared to serous carcinoma.

Molecular characterization of mucinous ovarian cancers has demonstrated that between approximately 20% of mucinous ovarian carcinomas are HER2+, as verified by both IHC and FISH criteria. See, e.g., Anglesio M, Kommoss S, Tolcher M, et al., Molecular Characterization of Mucinous Ovarian Tumors Supports a Stratified Treatment Approach with HER2 Targeting in 18% of Carcinomas. J. Pathol. 16:45-58 (2012); McAlpine J N, Wiegand K C, Vang R, et al., HER2 overexpression and amplification is present in a subset of ovarian mucinous carcinomas and can be targeted with trastuzumab therapy. BMC Cancer 9:433-441 (2009). A small series of women with HER2+ recurrent mucinous carcinoma that were concomitantly treated with trastuzumab demonstrated favorable response.

As the mucinous histotype of ovarian cancer is relatively treatment resistant and has a worse outcome than the other ovarian cancer subtypes, the identification of putative therapeutic target deserves further evaluation. Accordingly, subjects with HER2+ mucinous ovarian cancer could also be treated with an HER2+ inhibitor (e.g., trastuzumab or lapatinib) in combination with one or more cancer treating agents.

### III. The CA-125 and MUC16 Tumor Markers

CA-125 (cancer antigen 125) or Mucin 16 (MUC 16) is a protein that, in humans, is encoded by the MUC 16 gene. See, e.g., Yin B W, Dnistrian A, Lloyd K O. Ovarian cancer antigen CA125 is encoded by the MUC16 mucin gene. Int. J. Cancer 98(5):737-70 (2002). MUC 16 is a member of the mucin family glycoproteins. CA-125 has been identified as a tumor maker or biomarker whose levels may be elevated in the blood sera of some subjects with specific types of cancers.

Mucin 16 is a membrane associated mucin that possesses a single transmembrane domain and contains 22,000 amino acid residues, making it the largest membrane associated mucin protein. MUC 16 is made of three different domains; an N-terminal domain, a tandem repeat domain, and a C-terminal. The N-terminal domain and tandem repeat domain are both entirely extracellular and are highly O-glycosylated. The tandem repeat domain has repeating sequences high in serine, threonine, and proline residues. The C-terminal domain contains multiple extracellular SEA (sea urchin sperm protein, enterokinase, and agrin) modules, a transmembrane domain, and a cytoplasmic tail. The extracellular region of MUC16 can be released from the cell surface by undergoing proteolytic cleavage at a site thought to be located in the SEA modules.

CA-125 is the most frequently used biomarker for ovarian cancer detection. See, e.g., Suh K S, Park S W, Castro A, Patel H, Blake P, Liang M, Goy A. Ovarian cancer biomarkers for molecular biosensors and translational medicine. Expert Rev. Mol. Diagnostics 10(8):1069-1083 (2010). Approximately 90% of women with advanced ovarian cancer have been shown to possess elevated levels of CA-125 in their blood serum, making CA-125 a useful tool for detecting ovarian cancer after the onset of symptoms. See, Id. Monitoring CA-125 blood serum levels is also useful for determining how ovarian cancer is responding to treatment (with the duration of disease-free survival correlating with the rate of fall of CA-125) and for predicting a subject's prognosis following treatment. See, e.g., Santillan A, Garg R, Zahurak M L, Gardner G J, Giuntoli R L, Armstrong D K, Bristow R E. Risk of epithelial ovarian cancer recurrence in subjects with rising serum CA-125 levels within the normal range. J. Clin. Oncol. 23(36):9338-9343 (2005). In contrast, the persistence of high levels of CA-125 during therapy is associated with poor survival rates in subjects. See, Id. Similarly, an increase in CA-125 levels within individuals in a remission is a strong predictor of the recurrence of ovarian cancer. See, e.g., Santillan A, Garg R, Zahurak M L, Gardner G J, Giuntoli R L, Armstrong D K, Bristow R E. Risk of epithelial ovarian cancer recurrence in subjects with rising serum CA-125 levels within the normal range. J. Clin. Oncol. 23(36):9338-9343 (2005). Moreover, rising CA-125 levels may precede clinical evidence of disease relapse by an interval of 3 to 6 months.

Prognosis relates to both the initial and post-treatment CA-125 values. A pre-operative value >65 U/mL suggests a poor prognosis. Persistent elevations following cancer treatment agent therapy indicate a poor prognosis. The half-life of CA-125 after cancer treatment agent therapy correlates with prognosis (subjects with CA-125 half-life <20 days show improved survival). Time-to-normalization (rate of fall of CA-125) affects prognosis with more rapid normalization within 3 cycles of cancer treatment agent therapy correlating with improved survival. See, Mais D D, Leonard G R (2009). Quick Compendium Companion for Clinical Pathology (2nd ed.). Chicago: American Society for Clinical Pathology. p. 352.

In April 2011 the UK's National Institute for Health and Clinical Excellence (NICE) recommended that women with symptoms that could be caused by ovarian cancer should be offered a CA-125 blood test. The aim of this guideline is to help diagnose the disease at an earlier stage, when treatment is more likely to be successful. Women with higher levels of the marker in their blood would then be offered an ultrasound scan to determine whether they need further tests.

The potential role of CA-125 for the early detection of ovarian cancer is controversial and has not yet been adopted for widespread screening efforts in asymptomatic women. The major issues with using the CA-125 biomarker are its lack of sensitivity, particularly for detecting early stages of ovarian cancer, and its lack of specificity, especially in premenopausal women. See, e.g., Nossov V, Amneus M, Su F, Lang J, Janco J M, Reddy S T, Farias-Eisner R. The early detection of ovarian cancer: from traditional methods to proteomics. Can we really do better than serum CA-125? Am. J. Obstet. Gynecol. 199(3):215-223 (2008). These limitations mean that CA-125 testing often gives false positives for ovarian cancer and puts subjects through unnecessary further screening (sometimes including surgery) and anxiety. Also, these limitations mean that many women with early stage ovarian cancer may receive a false negative from CA-125 testing and not get further treatment for their condition.

Wang, et al. showed a male subject with IgE myeloma possessed elevated level of serum CA-125. See, Man-ling Wang, Qiang Huang, and Tian-xin Yang. IgE myeloma with elevated level of serum CA125. J. Zhejiang. Univ. Sci. B. 10(7):559-562 (2009).

CA-125 has limited specificity for ovarian cancer because elevated CA-125 levels can be found in individuals without ovarian cancer. For example, while CA-125 is best known as a marker for ovarian cancer, it may also be elevated in other cancers, including endometrial, fallopian tube, lung, breast, and gastrointestinal cancers. See, e.g., Bast R C, Xu F J, Yu Y H, Barnhill S, Zhang Z, Mills G B. CA 125: the past and the future. Int. J. Biol. Markers 13(4):179-187 (1998). CA-125 may also be elevated in a number of relatively benign conditions, such as endometriosis, several diseases of the ovary, menstruation, and pregnancy. It also tends to be elevated in the presence of any inflammatory condition in the abdominal area, both cancerous and benign. Thus, CA-125 testing is not perfectly specific for ovarian cancer and often results in false positives. The specificity of CA-125 is particularly low in premenopausal women because many benign conditions that cause fluctuations in CA-125 levels, such as menstruation, pregnancy, and pelvic inflammatory disease (PID), are seen in this population. Elevations in CA-125 can also be seen in cirrhosis and diabetes mellitus.

CA-125 testing is also not perfectly sensitive for detecting ovarian cancer because not every subject with cancer will have elevated levels of CA-125 in their blood. For example, 79% of all ovarian cancers are positive for CA-125, whereas the remainder do not express this antigen at all. See, e.g., Rosen D G, Wang L, Atkinson J N, Yu Y, Lu K H, Diamandis E P, Hellstrom I, Mok S C, Liu J, Bast R C. Potential markers that complement expression of CA125 in epithelial ovarian cancer. Gynecol. Oncol. 99(2):267-277 (2005). Also, only about 50% of subjects with early stage ovarian cancer have elevated CA-125 levels. Since many subjects with early stage ovarian cancer do not have elevated levels of CA-125, this biomarker has poor sensitivity for ovarian cancer, especially before the onset of symptoms.

While this test is not generally regarded as useful for large scale screening by the medical community, a high value may be an indication that the woman should receive further diagnostic screening or treatment. Normal values range from 0 to 35 U/mL. See, e.g., Nossov V, Amneus M, Su F, Lang J, Janco J M, Reddy S T, Farias-Eisner R. The early detection of ovarian cancer: from traditional methods to proteomics. Can we really do better than serum CA-125? Am. J. Obstet. Gynecol. 199(3):215-223 (2008). Elevated levels in post-menopausal women are usually an indication that further screening is necessary. In premenopausal women, the test is less reliable as values are often elevated due to a number of non-cancerous causes, and a value above 35 is not necessarily a cause for concern.

In a subject who is clinically selected for testing due to the presence of an adnexal/pelvic mass, CA-125 has great utility to differentiate benign from malignant processes. In a post-menopausal woman with a palpable adnexal mass and CA-125 level greater than 65 U/mL, the positive predictive value is >95% for ovarian malignancy. In subjects who are not as carefully selected clinically, the utility of this test decreases, thus highlighting the need for careful clinical scrutiny.

MUC16 has been shown to play a role in advancing tumorigenesis and tumor proliferation by several different mechanisms. One mechanism by which MUC16 aids in the growth of tumors is by suppressing the response of Natural Killer Cells, thus protecting cancer cells from the immune response. See, e.g., Patankar M S, Jing Y, Morrison J C, Belisle J A, Lattanzio F A, Deng Y, Wong N K, Morris H R, Dell A, Clark G F. Potent suppression of natural killer cell response mediated by the ovarian tumor marker CA125. Gynecol. Oncol. 99(3):704-713 (2005). Further evidence of the role of MUC16 in allowing tumor cells to evade the immune system is the discovery that the heavily glycosylated tandem replete domain of MUC16 can bind galectin-1, an immunosuppressive protein.

MUC16 is also thought to participate in cell-to-cell interactions that allow for the metastasis of tumor cells. This is supported by evidence showing that MUC16 binds selectively to mesothelin, a glycoprotein normally expressed by the mesothelial cells of the peritoneum. MUC16 and mesothelin interactions are thought to provide the first step in tumor cell invasion of the peritoneum. See, e.g., Rump A, Morikawa Y, Tanaka M, Minami S, Umesaki N, Takeuchi M, Miyajima A. Binding of ovarian cancer antigen CA125/MUC16 to mesothelin mediates cell adhesion. J. Biol. Chem. 279(10):9190-9198 (2005). Mesothelin has also been found to be expressed in several types of cancers including mesothelioma, ovarian cancer and squamous cell carcinoma. Since mesothelin is expressed by tumor cells, MUC16 and mesothelial interactions may aid in the gathering of other tumor cells to the location of a metastasis, thus increasing the size of the metastasis. See, Id.

Evidence suggests that the cytoplasmic tail of MUC16 enables tumor cells to grow and become motile and invasive. This appears to be due to the ability of the C-terminal domain of MUC16 to decrease the expression of E-cadherin and increase the expression of N-cadherin and vimentin, which are expression patterns consistent with epithelial-mesenchymal transition. See, e.g., Thériault C, Pinard M, Comamala M, Migneault M, Beaudin J, Matte I, Boivin M, Piché A, Rancourt C. MUC16 (CA125) regulates epithelial ovarian cancer cell growth, tumorigenesis and metastasis. Gynecol. Oncol. 121(3):434-443 (2011).

MUC16 may also play a role in reducing the sensitivity of ovarian cancer tumor cells to drug therapy. Overexpression of MUC16 has been shown to protect cells from the effects of genotoxic drugs, such as cisplatin. See, e.g., Boivin M, Lane D, Piché A, Rancourt C. CA125 (MUC16) tumor antigen selectively modulates the sensitivity of ovarian cancer cells to genotoxic drug-induced apoptosis. Gynecol. Oncol. 115(3):407-413 (2009).

### III. Current Treatment Options for Advanced Ovarian Cancer

The probability for cure for subjects with advanced ovarian cancer had previously been thought to be remote, with palliation and optimizing the quality of life being the primary treatment goals (see, e.g., Gordon A N, Fleagle J T, Guthrie D, Parkin D E, Gore M E, Lacave A J. Recurrent epithelial ovarian carcinoma: a randomized phase III study of pegylated liposomal doxorubicin versus Topotecan. J. Clin. Oncol. 19(14):3312-3322 (2001)). Accordingly, the observations in the instant Karenitecin Phase III Trial regarding the ability of the subjects receiving Karenitecin: (i) to tolerate the full treatment cycle; (ii) to tolerate a greater number of chemotherapeutic cycles; and (iii) to have a reduction in cancer treating agent-related toxicities, may markedly improve the probability of advanced ovarian cancer being able to be treated as a chronic disease or even for a cure.

The existing recommended treatment strategy for subjects with advanced-stage ovarian cancer (stage III/IV) includes cytoreductive surgery (i.e., removal of all visible tumor) followed by platinum- and/or taxane-based cancer treating agent therapy—generally cisplatin or carboplatin with paclitaxel. See, e.g., Karlan B Y, Markman M A, Eifel P J. Ovarian cancer, peritoneal carcinoma, and fallopian tube carcinoma. In: DeVita V T Jr, Hellman S, Rosenberg S A, eds. Cancer. Principles & Practice of Oncology. 9th ed. Philadelphia, Pa.: Lippincott Williams & Wilkins; 2011:1368-1391. While almost 80% of previously-untreated subjects with advanced stage disease achieve a complete clinical remission (CR) after platinum and taxane cancer treating agent therapy; between 50% and 75% of subjects with advanced-stage disease ultimately experience relapse. Even subjects who are surgically-confirmed to be in complete remission still remain at high-risk, with a relapse rate of 30% to 50% after platinum-based cancer treating agent therapy. See, Id.

The treatment strategy for subjects with recurrent ovarian cancer is based upon the initial cancer treating agent regimen used and on the initial response to treatment. Patients who respond to a platinum-based cancer treating agent regimen and then experience a relapse after a disease-free interval of more than 6 months are considered platinum-sensitive (i.e., having a likelihood of achieving a secondary response, wherein said likelihood increases as the duration of disease-free interval increases); and are retreated with a platinum-based cancer treating agent regimen. A single-agent carboplatin regimen is the currently preferred platinum compound for treatment of such platinum-sensitive recurrent disease. See, Id.

Although platinum-sensitive subjects are frequently considered as candidates for re-treatment with regimens similar to those received in the first-line setting, there is no evidence from prospective, randomized trials that combination multi-agent cancer treatment regimens achieve superior outcomes in terms of survival or quality of life compared to the use of sequential single agents. In addition, early re-treatment with platinum places the subject at risk for cumulative hematologic and non-hematologic toxicity that can limit further therapy and diminish the overall quality of life. Most subjects, however, eventually develop platinum-resistance, and salvage cancer treating agent therapy is much less effective than first-line therapy. See, e.g., Piccart M J, Green J A, Lacave A J, et al. Oxaliplatin or paclitaxel in subjects with platinum pretreated advanced ovarian cancer: a randomized phase II study of the European Organization for Research and Treatment of Cancer Gynecology Group. J Clin Oncol. 2000; 18(6):1193-1202. In some subjects, the use of a non-platinum regimen may extend the platinum-free interval with less risk of cumulative toxicity. See, e.g., Markman M, Bookman M A. Second-line treatment of ovarian cancer. The Oncologist 5:26-35 (2000).

### IV. Treatment of Platinum/Taxane-Resistant Ovarian Cancer

The optimal therapeutic approach for the treatment of relapsed, advanced ovarian cancer would be based upon an understanding of the molecular genetics and biology of the disease; as well as the limitations of the initial therapy utilized. See, e.g., Ozols R F. Treatment of recurrent ovarian cancer: increasing options—“recurrent” results. J. Clin. Oncol. 15:2177-2180 (1997). As previously discussed, the current standard of care for newly diagnosed advanced-stage ovarian cancer includes cytoreductive surgery followed by combination cancer treating agent therapy with platinum (i.e., either cisplatin or carboplatin) and paclitaxel. However, despite the high overall clinical response rates achieved with platinum-based therapy (up to 80%; including a high proportion of complete responses), most subjects subsequently relapse and require additional cancer treating agent treatment. See, Id. The primary goal of this subsequent second-line therapy is to control of disease so as to maintain quality of life and extend survival. Therapeutic options include retreatment with platinum and/or paclitaxel, although it should be noted that subjects retreated with carboplatin and paclitaxel are at increased risk from cumulative hematologic toxicity (e.g., anemia, thrombocytopenia, neutropenia). Other treatment options in the relapsed setting include initiation of a non-cross-resistant cancer treating agent or the use of investigational agents in the context of a clinical trial.

The outcome of second-line cancer treating agent therapy in relapsed ovarian cancer can be partially predicted by whether the disease is drug-sensitive (i.e., response duration more than six months) or drug-resistant (i.e., response duration less than six months). See, e.g., McGuire W P, Ozols R F. Chemotherapy of advanced ovarian cancer. Semin. Oncol. 25:340-348 (1998). Patients with tumors classified as drug sensitive have demonstrated relatively high response rates (40-50%) to second-line platinum-based therapy. The time that elapses after the completion of the initial platinum-based therapy is defined as the platinum-free interval. In general, the longer the platinum-free interval, the higher the response rate to subsequent retreatment of the subject. In contrast, subjects who do not respond to a platinum- and/or taxane-based cancer treating agent regimen or who relapse within 6 months after completing a platinum- and/or taxane-based regimen are considered to be refractory or resistant to platinum and/or taxane cancer treating agents; and are generally not re-treated with these same regimens, as they have response rates to second-line platinum-based therapy as low as 10%. It should be noted, that the aforementioned difference between drug-sensitive and drug-resistant tumors was initially described in relationship to platinum-based therapy, but is also applicable to other cancer treating agent regimens.

Extending the platinum-free interval in recurrent ovarian cancer after first relapse by using an alternative cancer treating agent regimen may help to increase the response to platinum analogue drugs that may be subsequently administered at the time of further disease progression. The availability of newer non-cross-resistant cancer treating agents (which will be discussed infra) has expanded the treatment options for subjects with relapsed disease. By way of non-limiting example, the camptothecin derivative, topotecan, has demonstrated efficacy comparable to paclitaxel across all categories of platinum sensitivity and exhibited activity in subjects with ovarian cancer which has shown to be resistant to both platinum and paclitaxel. See, e.g., ten Bokkel Huinink W, Gore M, Carmichael J, et al., Topotecan versus paclitaxel for the treatment of recurrent epithelial ovarian cancer. J. Clin. Oncol. 15(6):2183-2193 (1997).

Although many clinicians recommend early retreatment with platinum and paclitaxel, this approach has not been shown to be superior to the use of non-platinum-based therapy and places the subject at greater risk for cumulative non-hematologic toxicity.

A. Platinum Cancer Treating Agent Drug Sensitivity

There are a number of indicators which can be utilized in prognostic manner in relapsed ovarian cancer including, but not limited to: (i) performance status; (ii) tumor volume; (iii) tumor histology; and (iv) CA-125 levels. See. Table I; Thigpen J T, Vance R B, Khansur T. Second-line chemotherapy for recurrent carcinoma of the ovary. Cancer 71:1559-1564 (1993).

TABLE I
Prognostic factors in relapsed ovarian cancer

| Poor Prognosis | Good Prognosis |
|---|---|
| Disease-free (treatment-free) | Disease-free (treatment-free) |
| interval <6 months | interval >6 months |
| Poor performance status | Good performance status |
| (Zubrod 3-4/Karnofsky 10-40) | (Zubrod 1-2/Karnofsky 50-100) |
| Serum CA-125 level >35 U/ml | Serum CA-125 level <35 U/ml |
| Multiple disease sites/large tumor | Single disease site/small tumor |
| volume | volume |
| Mucinous or clear cell tumor | Papillary serous histology |
| histology | |

Platinum sensitivity, defined herein as the disease-free or treatment-free interval, has emerged as an important and significant predictor of response to second-line cancer treating agent therapy in a number of clinical studies conducted over the past two decades. See, e.g., Kavanagh H, Tresukosol D, Edwards C., et al., Carboplatin reinduction after taxane in subjects with platinum-refractory epithelial ovarian cancer. J. Clin. Oncol. 13:1584-1588 (1995). Patients who responded to first-line therapy and demonstrated a significant treatment-free interval also had a high probability of responding again to platinum-based treatment. See, e.g., Thigpen J T, Vance R B, Khansur T. Second-line chemotherapy for recurrent carcinoma of the ovary. Cancer 71:1559-1564 (1993).

Although there has been variability in the definition of platinum sensitivity in the literature, subjects can generally be stratified into three categories. The first category is platinum-sensitive disease, which includes subjects who have relapsed more than six months after completing prior platinum therapy, usually in association with a clinical complete remission. Within this heterogeneous group, subjects who have been platinum-free (and disease-free) for more than two years have the greatest likelihood of response and are often retreated with a combination of platinum and paclitaxel. See, e.g., McGuire W P, Ozols R F. Chemotherapy of advanced ovarian cancer. Semin. Oncol. 25:340-348 (1998). Those subjects who relapse between six and 24 months after first-line therapy are often treated with a new second-line agent, or retreated with platinum and/or paclitaxel, although the likelihood of achieving a long-term remission is diminished. Platinum-resistant disease includes subjects that have relapsed within six months of prior platinum therapy. As a group, the expected response rate to retreatment with platinum is less than 20%, although some subjects may remain platinum-sensitive. See, e.g., Markman M. Recurrence within 6 months of platinum therapy: an adequate definition of “platinum-refractory” ovarian cancer? Gynecol. Oncol. 69:91-92 (1998). The third category is platinum-refractory disease, which can be defined as disease that progressed or was stable during prior platinum therapy.

B. The Importance of the Platinum-Free Interval

In second-line and salvage treatment settings, the probability of response to retreatment with platinum-based cancer treating agents and compounds and various other agents increases as the platinum-free and treatment-free intervals increase. This has been well documented in the medical literature.

Some of the seminal studies examining relapsed ovarian cancer and the impact of the platinum-free interval include:

(i) Blackledge G, Lawton F, Redman C., et al., Response of subjects in phase II studies of chemotherapy in ovarian cancer: implications for subject treatment and the design of phase II trials. Br. J. Cancer 59:650-653 (1989)—Univariate and multivariate analyses of a total of five phase II clinical studies, established that the platinum-free interval was the most significant variable in predicting the response to second-line cancer treating agent therapy. Specifically, among 92 subjects with relapsed ovarian cancer, 62% of subjects with a treatment-free interval of seven months were found to respond to second-line cancer treating agent therapy, compared with only 10% of subjects with a treatment-free interval of six months. Patients with a treatment-free interval of >21 months had the highest response rates, 94%, to second-line therapy.
(ii) Gore M E, Fryatt I, Wiltshaw E, et al., Treatment of relapsed carcinoma of the ovary with cisplatin or carboplatin following initial treatment with these compounds. Gynecol. Oncol. 36:207-211 (1990)—A study of ovarian cancer subjects who subsequently relapsed following initial treatment with cisplatin or carboplatin and were either crossed over to the other platinum compound (n=43) or retreated with the same drug (n=11). Similarly, the progression-free interval was a highly significant prognostic variable for response to treatment. Among subjects who relapsed 18 months after the end of initial platinum-based therapy, 53% responded to crossover treatment or retreatment. In contrast, among those subjects who relapsed within 18 months, only 17% responded (p=0.006).
(iii) Markman M, Rothman R, Hakes T, et al., Second-line platinum therapy in subjects with ovarian cancer previously treated with cisplatin. J. Clin. Oncol. 9:389-393 (1991)—A retrospective analysis of subjects who relapsed after initial platinum-based therapy (n=82). Patients had a platinum-free interval of 5 to 12 months (n=26), 13 to 24 months (n=23), or >24 months (n=33). In addition, 29 subjects received a course of non-platinum-based therapy between platinum regimens. The overall response rate for the 72 assessable subjects was 43%, which included 10 complete and 21 partial responses. When results were analyzed according to the platinum-free interval, response rates increased with greater distance from the initial therapy. The difference in response rates between a platinum-free interval of >24 months versus <24 months was statistically significant (p<0.025). Response rates were also examined according to the treatment-free interval, defined as the time elapsed after the last anti-neoplastic treatment (regardless of the drugs used) and initiation of second-line platinum therapy. Similarly, the response rate was greatest in subjects with the longest treatment-free interval.
(iv) Kavanagh H, Tresukosol D, Edwards C, et al., Carboplatin reinduction after taxane in subjects with platinum-refractory epithelial ovarian cancer. J. Clin. Oncol. 13:1584-1588 (1995)—An important study which demonstrated that extending the platinum-free interval in relapsed ovarian cancer by using a non-platinum agent is associated with higher responses to platinum retreatment. In this study, subjects had originally received the two drug combination of cyclophosphamide and cisplatin or carboplatin. A total of 33 subjects with platinum-refractory disease subsequently relapsed or progressed on a taxane (paclitaxel or docetaxel) and were then retreated with single-agent carboplatin. The median time from previous platinum-based therapy to platinum reinduction after taxane failure was 15 months (with a range of 8 to 33 months); there were 26 subjects with a platinum-free interval of 12 months. The overall response rate to platinum reinduction was 21%, with 7/33 subjects achieving a partial response. Another 18% (6 subjects) had stable disease. The median duration of response was 7+ months. Responses were noted only in subjects with a platinum-free interval of 12 months and initial taxane sensitivity; whereas no responses were noted in any subject who was taxane-resistant. The response rate for the subgroup of 26 subjects with a platinum-free interval of 12 months was 27% (7/26). Of clinical importance were three subjects who had progressed on initial platinum treatment before taxane therapy and who then achieved partial responses to carboplatin reinduction. Kavanagh and colleagues suggested that since all of the subjects who responded to carboplatin reinduction were taxane-sensitive: (a) taxane exposure may have eliminated the platinum-resistant clone and/or (b) the prolonged platinum-free interval may have resulted in loss of acquired platinum resistance. Therefore, they surmised that the likelihood of achieving a response in subjects with initially platinum-sensitive tumors increases when the interval from the last cycle of initial platinum-based treatment is longer than 12 months. That time interval may allow for the regrowth of platinum-sensitive cells or for resistant cells to lose their resistance to the cytotoxic drugs.

As previously discussed, the combination of a platinum analogue and paclitaxel has become the standard of care for the management of newly diagnosed advanced disease. However, even with paclitaxel-based regimens, the majority of subjects will eventually relapse and develop drug-resistant disease that is resistant to the paclitaxel and/or other drugs utilized in such regimen. When relapse occurs, it has been common practice to retreat platinum-sensitive subjects using a combination of platinum and paclitaxel, and high response rates have been reported. See, e.g., Rose P G, Fusco N, Fluellen L. et al., Second-line therapy with paclitaxel and carboplatin for recurrent disease following first-line therapy with paclitaxel and platinum in ovarian or peritoneal carcinoma. J. Clin. Oncol. 16:1494-1497 (1998). However, both cisplatin and carboplatin are associated with potential long-term hematologic and non-hematologic toxicities that may limit the amount and/or duration of therapy. Recurrent ovarian cancer is generally incurable, and therapeutic strategies need to be integrated with maintenance of quality of life and associated endpoints. In this regard, the use of non-platinum-based therapies to extend the platinum-free interval and reduce the impact of cancer treating agent-related side effects and toxicities may maintain a higher quality of life and improve the likelihood of responding to subsequent retreatment with platinum with better tolerability.

Cancer treating agents that have been shown to have some activity in subjects with platinum- and/or paclitaxel-resistant ovarian cancer include, but are not limited to, Topotecan, oral etoposide, gemcitabine, liposomal doxorubicin, vinorelbine, and altretamine. See, e.g., Karlan B Y, Markman M A, Eifel P J. Ovarian cancer, peritoneal carcinoma, and fallopian tube carcinoma. In: DeVita V T Jr, Hellman S, Rosenberg S A, eds. Cancer. Principles & Practice of Oncology. 9th ed. Philadelphia, Pa.: Lippincott Williams & Wilkins; 2011:1368-1391. Drugs that are currently approved by the United States Food and Drug Administration (FDA) for the treatment of recurrent ovarian cancer include: (i) Taxol (paclitaxel, Bristol-Myers Squibb Company); (ii) Hycamtin (Topotecan, GlaxoSmithKline); (iii) Doxil (doxorubicin HCl liposomal, Janssen); and (iv) Gemzar (gemcitabine, Eli Lilly) administered in combination with carboplatin.

Doxorubicin liposomal-formulation (Doxil) is an anthracycline antibiotic, closely related to the natural product daunomycin. Like all anthracyclines, it works by intercalating DNA. The drug is administered intravenously, as the hydrochloride salt. It may also be sold under the brand names Adriamycin PFS, Adriamycin RDF, or Rubex. Doxil is typically administered one a month at a dose of 50 mg/m² of body surface area, at a rate of approximately 1 mg/min. The most serious adverse effect of doxorubicin administration is life-threatening heart damage and/or heart conditions (e.g., congestive heart failure, cardiac arrhythmias, and the like). See, e.g., Sneader, Walter (2005). Drug Discovery: A History. New York: Wiley. p. 467.

Gemcitabine (Gemzar) is a nucleoside analog in which the hydrogen atoms on the 2′ carbon of deoxycytidine are replaced by fluorine. As with fluorouracil and other analogues of pyrimidines, the triphosphate analogue of gemcitabine replaces one of the nucleic acid building blocks, in this case cytidine, during DNA replication. This replacement process arrests tumor growth, as only one additional nucleoside can be attached to the “faulty” nucleoside, resulting in cellular apoptosis. Another target of gemcitabine is the enzyme ribonucleotide reductase (RNR). The diphosphate analogue binds to RNR active site and irreversibly inactivates the enzyme. Once RNR is inhibited, the cell cannot produce the deoxyribonucleotides required for DNA replication and repair; thus cellular apoptosis is induced. Gemcitabine is administered by the intravenous route, as it is extensively metabolized by the gastrointestinal tract. Doses range from 1-1.2 g/m² of body surface area, according to type of cancer treated. The most commonly reported serious adverse effects were hematologic in nature, with neutropenia occurring in up to 90% of subjects. See, e.g., Sneader, Walter (2005). Drug Discovery: A History. New York: Wiley. p. 259.

The camptothecin, Topotecan, is a treatment option for subjects with advanced epithelial ovarian cancer. See, e.g., Herzog T J. Update on the role of Topotecan in the treatment of recurrent ovarian cancer. The Oncologist. 7:3-10 (2002). The principal toxicity of Topotecan is myelosuppression, which may limit its use in platinum-resistant subjects due to often incomplete bone marrow recovery following previous platinum treatment.

The silicon-containing HLCD, Karenitecin, was specifically developed to markedly improve key limitations of other camptothecins, which include: (i) safety; (ii) antitumor activity; (iii) potency; (iv) metabolism; (v) bioavailability; and (vi) sensitivity to multi-drug resistance proteins. Additionally, Karenitecin is at least 600-fold less water soluble than camptothecin. Karenitecin has demonstrated clinical activity that appears to be superior to that of Topotecan. Furthermore, the safety profile of Karenitecin suggests a reduced incidence of severe (NCI-CTCAE≧grade 3) hematologic toxicity. This is of particular importance since an improved hematologic toxicity profile may reduce the need for frequent monitoring of bone marrow function and treatment interventions (e.g., treatment delays, dose reductions, red blood cell [RBC] transfusions, growth factor support, and the like), thus improving subject safety and compliance, as well as the overall clinical benefit.

Results from three Phase I studies indicate that Karenitecin can be safely administered to subjects at the dose level of 1.0 mg/m²/day i.v. over a one hour period of time for 5 consecutive days in a 3-week treatment cycle. The principal and dose-limiting toxicity is non-cumulative, reversible myelosuppression. Any gastrointestinal toxicity is generally ≦grade 2 and is not dose-limiting.

In four Phase II studies, Karenitecin demonstrated a good safety profile and evidence of clinical activity in subjects with metastatic melanoma, advanced ovarian and peritoneal cancer, malignant glioma, and advanced non-small cell lung cancer (NSCLC).

The Phase II study in subjects with advanced ovarian cancer who had failed prior treatments demonstrated potential efficacy outcomes with Karenitecin as evidenced by prolonged Time to Progression (TTP) as compared with historical results reported with Topotecan.

It should be noted that the aforementioned previously performed pre-clinical studies and earlier-stage clinical trials were of importance in the determination and development of both the actual cancer treating agent drug regimen for the recently conducted Phase III clinical trial. The data from the aforementioned pre-clinical and clinical trials had to be collected and carefully analyzed by the Inventors of the present patent application in order to seek and obtain permission from the various regulatory agencies (i.e., the FDA) to engage in more advanced clinical studies (e.g., Phase II→Phase III) with increased complexity and substantially greater numbers of subjects. Conducting the Phase III clinical trial was critical for the inventions disclosed in the instant patent application in order to allow the evaluation of Karenitecin in this specific and rigorously selected subject population with a larger number of subjects in which detailed measurements of PFS, total number of subject treatment cycles, safety related events, and other important clinical observations could be made in a larger subject population. By way of non-limiting example, PFS was radiographically determined by an Independent Radiological Committee (IRC). Additionally, highly specific or advanced methodologies were used to, e.g., initially select, diagnosis, and/or define the subject population of the instant Phase III clinical trial. As described herein, the subject population selected for inclusion in the instant Phase III clinical trial were all refractory and/or resistant to a platinum- and/or taxane-based cancer treating agent regimen. In addition, the Karenitecin Phase III clinical trial disclosed herein was designed to allow subjects to continue receiving the study treatment until such time as their disease progressed and was also designed to carefully monitor the clinical study treatment cycles.

### IV. Discovery and Initial Development of Camptothecin

In the late 1950s, CPT was isolated from Camptotheca acuminata, a tree native to China, its chemical structure was characterized, and evidence of potent antitumor activity was documented. See, e.g., Wall, M. E., et al., Plant antitumor agents. I. The isolation and structure of camptothecin, a novel alkaloidal leukemia and tumor inhibitor from Camptotheca acuminata. J. Am. Chem. Soc. 88:3888-3890 (1966). Although the naturally-occurring CPTs possessed antitumor activity, product formulation and delivery were problematic due to poor water solubility. As a means to address the poor water solubility, the water-soluble sodium salt form (or carboxylate form) of CPT was used in early clinical trials.

The structure of this originally isolated camptothecin (CPT) is shown below:

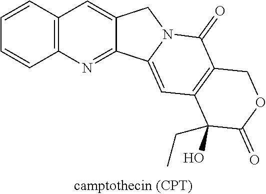 [SMILES: CC[C@@]1(O)C(=O)OCc2c1cc1n(c2=O)Cc2cc3ccccc3nc2-1]

These early clinical studies produced disappointing results in terms of both efficacy and safety by demonstrating substantially less antitumor activity than expected, and severe and unpredictable toxicity including hemorrhagic cystitis, diarrhea, and myelosuppression. See, e.g., Muggia, F. M., et al., Phase I clinical trial of weekly and daily treatment with camptothecin (NSC-100880): correlation with preclinical studies. Cancer Chemother. Rep. 56(4):515-521 (1972).

It was later discovered that an intact 20(S) lactone ring (i.e., lactone form) plays an essential role in the observed antitumor activity of CPTs; and that hydrolysis of the CPT lactone ring yields a carboxylate form of the molecule which has substantially lower antitumor activity. Because of its critical role in the antitumor activity of CPTs, the stability of the 20(S) lactone ring has been studied extensively in a variety of CPTs. See, e.g., Giovanella, B. C., et al., Dependence of anticancer activity of camptothecins on maintaining their lactone function. In: Liehr, J. G., Giovanella, B. C., and Verschraegen, C. F., eds. The Camptothecins. Unfolding Their Anticancer Potential. Ann. New York Acad. Sci. 922:27-35 (2000).

### V. Biochemical and Molecular Pharmacology of Camptothecins

A. Topoisomerase I

Topoisomerase I, the target enzyme of the camptothecins, is a 100 kDa protein composed of 765 amino acids. The enzymatic activity of topoisomerase I is found in a 67.7 kDa region located at the carboxyl-terminal end of the protein. The topoisomerase I gene is located on human chromosome 20, and it consists of 21 exons extending over 85 kilobases of DNA. Expression of topoisomerase I is found in nearly all mammalian cells at a high copy number, estimated at approximately 10⁶ per cell. See, e.g., Kunze, N., et al., The structure of the human type I DNA topoisomerase gene. J. Biol. Chem. 266:9610-9615 (1991).

Topoisomerase I acts to relax supercoiled double-stranded DNA, a function it partially shares with the related enzyme topoisomerase II. Unwinding of the DNA helix is essential for normal DNA function such as DNA replication or RNA transcription. This unwinding generates torsional strains in the DNA resulting form supercoiling of the helix above and below the region of ongoing nucleic acid synthesis. Topoisomerase I relaxes both positively- and negatively-supercoiled DNA and allows these functions to proceed in an orderly fashion. Although its exact role has not been fully elucidated, the involvement of topoisomerase I in RNA transcription has been postulated. High levels of topoisomerase I have been localized by immunohistochemical methods to regions of the nucleus that are active in RNA transcription, such as the nucleolus. See, e.g., Muller, M., et al., Eukaryotic type I topoisomerase is enriched in the nucleolus and catalytically active on ribosomal DNA. EMBO J. 4:1237 (1988). Although little is known about the regulation of topoisomerase I activity, phosphorylation by protein kinase C appears to activate the enzyme.

Unlike other topoisomerases, topoisomerase I is constitutively expressed at relatively constant levels throughout the cell cycle, even in cells that are actively dividing. Thus inhibitors of topoisomerase I, such as the camptothecins, may potentially be active in tumors that have low growth fractions and are resistant to other anticancer agents. In comparative studies, higher levels of topoisomerase I protein and mRNA were found in malignant colon and prostate tumors relative to their normal counterparts. See, e.g., Hussain, I, et al., Elevation of topoisomerase I messenger RNA, protein, and catalytic activity in human tumors. Cancer Res. 54:539 (1994). Consequently, the camptothecins may be selectively toxic to tumor cells compared with normal tissues.

B. Topoisomerase Molecular Function

Many of the steps involved in the topoisomerase I-mediated reaction that relaxes supercoiled DNA have been characterized at the molecular level. Topoisomerase I preferentially binds to supercoiled double-stranded DNA and cleaves the phosphodiester bond, resulting in a single-strand nick. During this process, the topoisomerase I enzyme is temporarily bound by a covalent bond between a tyrosine residue at position 723 and the 3′-terminus of the single-strand break in the DNA. This normally short-lived intermediate has been called the cleavable complex, and once it has been formed, free rotation of the DNA molecule can occur about the remaining intact phosphodiester bond, allowing for the relaxation of the torsional strain in the DNA. Finally, relegation of the strand break restores the integrity of the double-stranded DNA, and the enzyme dissociates from the now relaxed double helix. Typically, this reaction is very rapid, and topoisomerase I protein bound to DNA cannot be isolated under normal conditions.

C. Mechanism of Action of the Camptothecins

In the presence of camptothecins, the topoisomerase I enzymatic reaction is altered, resulting in a drug-induced stabilization of the cleavable complex. See, e.g., Potmesil, M., Camptothecins from bench research to hospital ward. Cancer Res. 54:1431 (1994). Camptothecins interact non-covalently with the DNA-bound topoisomerase I and inhibit the relegation step of the reaction. Consequently, there is accumulation of stabilized cleavable complexes and a persistence of single-stranded DNA breaks. However, this DNA damage alone is not toxic to the cell, because the lesions are highly reversible and can be repaired rapidly once the drug is removed. Instead, ongoing DNA synthesis is required in order to convert these stabilized cleavable complexes into more lethal DNA damage. See, e.g., D'Arpa, P., et al., Involvement of nucleic acid synthesis in cell killing mechanisms of topoisomerase poisons. Cancer Res. 50:6919 (1990). According to the “fork collision model,” irreversible damage to the DNA occurs only when a DNA replication fork encounters a cleavable complex, resulting in the formation of a complete double-stranded break in the DNA. See, e.g., Tsao, Y. P., et al., Interaction between replication forks and topoisomerase I DNA cleavage complexes: studies in a cell-free SV-40 replication system. Cancer Res. 53:5908 (1993). Thus, the cleavable complexes are necessary, but not sufficient for drug toxicity. In support of this theory are observations that inhibitors of DNA synthesis, such as aphidicolin or hydroxyurea, can protect cells from camptothecin-induced cytotoxicity. If ongoing, DNA synthesis is truly necessary for drug-induced toxicity, then the camptothecins should be highly S-phase cell-cycle-specific in their action. This finding has been confirmed in most, but not all experimental studies. This point has important implications for the design of clinical therapeutic regimens, because highly S-phase-specific cytotoxic agents require prolonged exposures to drug concentrations above a minimum threshold in order to maximize the fractional cell kill.

Although the camptothecins can clearly produce irreversible DNA damage in the presence of ongoing DNA synthesis, the events responsible for cell death once these lesions occur have been elucidated fully. The camptothecins, as well as other DNA-damaging agents, can cause cell-cycle arrest, typically in the G₂ phase. The molecular mechanisms responsible for regulation this block in the cell cycle have been examined. Camptothecin-induced DNA damage correlates with altered activity of the p34^{cdc2}/cyclin B complex, which has been tightly linked to regulation of the G₂ to M-phase transition in the cell cycle. See, e.g., Tsao, Y. P., et al., Interaction between replication forks and topoisomerase I DNA cleavage complexes: studies in a cell-free SV-40 replication system. Cancer Res. 53:5908 (1993). The relevance of this G₂ block to cytotoxicity is not clear. Failure of cells to arrest at the G₂ checkpoint following drug treatment may be associated with increase camptothecin toxicity. A preliminary report suggests that the differential sensitivity of various cell lines to the camptothecins inversely correlates with their ability to undergo G₂ arrest following drug exposure. See, e.g., Goldwasser, F., et al., Integrity of G₂ checkpoint is a critical determinant for sensitivity to camptothecin. In: Programs and Abstracts. The Fifth Conference on DNA Topoisomerases in Therapy. New York, pg. 51 (1994). Other reported actions of the camptothecins include the induction of differential in human promonocytic leukemia cells and the increased expression of the c-jun early-response gene. Finally, the camptothecins cytotoxicity also has been associated with the endonucleolytic degradation of DNA, resulting in a pattern of DNA fragmentation similar to that described for programmed cell death or apoptosis. Id. Further studies on the nature of cellular events that occur as a result of camptothecin-induced DNA damage are required.

Camptothecins also can damage DNA at the chromosomal level. Dose-dependent increases in sister chromatid exchange (SCE) and chromosomal aberrations were detected in the peripheral blood lymphocytes obtained from subjects following irinotecan treatment. See, e.g., Kojima, A., et al., Cytogenetic effects of CPT-11 and its active metabolite, SN-38, on human lymphocytes. Jpn. J. Clin. Oncol. 23:116 (1993). The chromosomal damage was manifested mainly by chromatid gaps and breaks. Although little is known concerning the possible long-term side effects of camptothecin therapy, other DNA-damaging agents, such as alkylating agents, have been associated with mutagenicity. Additional study will be required to determine whether these risks are also relevant to the camptothecins.

The presence of topoisomerase I is essential for the generation of camptothecin-induced cytotoxicity. Mutant yeast cells that lack functional topoisomerase I are completely resistant to the camptothecins. However, when topoisomerase I is transfected into these mutants, drug sensitivity is restored. See, e.g., Nitiss, J. and Wang, J. C., DNA topoisomerase targeting antitumor drugs can be studied in yeast. Proc. Natl. Acad. Sci. U.S.A. 85:7501 (1988). These experiments illustrate how camptothecin's mechanism of action differs from the more traditional pharmacologic inhibition of an essential enzyme. In order to generate drug toxicity, complete inhibition of topoisomerase I is not necessary or even required. Instead, the camptothecins generate drug toxicity by converting a normally functioning constitutive protein, topoisomerase I, into a cellular poison.

D. Cleavage Site Sequence Specificity

Topoisomerase I cleavage is not a random event and the single-strand nicks appear with increase frequency at specific sequence sites in the DNA. See, e.g., Jaxel, C., et al., Effect of local DNA sequence on topoisomerase I cleavage in the presence or absence of camptothecin. J. Biol. Chem. 266:20418 (1991). Interestingly, camptothecin does not stabilize all topoisomerase I cleavable complexes equally. Instead, enhanced stabilization of cleavage sites by camptothecin occurs when a guanine residue is immediately 3′ to the phosphodiester bond normally cleaved by the enzyme. In the absence of drug, topoisomerase I has no specific base preference at this location, suggesting that only a subset of the total topoisomerase I cleavage sites is stabilized by camptothecin. This has led to a proposed camptothecin stacking model in which the drug specifically interacts with guanine residues at the topoisomerase I-DNA cleavage site. Preliminary findings also suggest that various camptothecin derivatives may stabilize different topoisomerase I cleavage sites. See, e.g., Tanizawa, A., et al., Comparison of topoisomerase I inhibition, DNA damage, and cytotoxicity of camptothecin derivatives presently in clinical trials. J. Natl. Cancer Inst. 86(11):836 (1994). An in-depth understanding of these site-specific interactions will be greatly facilitated by crystallographic characterization of the molecular structure of the camptothecin-stabilized cleavable complex.

E. Mechanisms of Camptothecin Drug Resistance

Although little is known about the mechanisms of camptothecin resistance in human tumors, in vitro camptothecin resistance has been characterized in several different cell lines. Single-base mutations in the topoisomerase I enzyme can decrease its interaction with the camptothecins, resulting in drug resistance. Recently, several different amino acid substitutions have been characterized in human topoisomerase I that confer a relative resistance to the camptothecins. These substitutions span a large portion of the protein, and they include changing a glycine 363 to cysteine, threonine 729 to alanine, and phenylalanine 301 to serine or aspartic acid 533 or 583 to glycine. See, e.g., The Camptothecins. In: Cancer Chemotherapy and Biotherapy, 2^{nd} Edition. B. A. Chabner and D. L. Longo, eds. Lippincott-Raven Publishing New York, N.Y. (1996). Further research is likely to identify additional topoisomerase I mutants with relative camptothecin resistance. Insensitivity to the camptothecins also can result from decreased expression of topoisomerase I. In an in vitro study, a resistant subline of cells containing less than 4% of the topoisomerase I activity compared with wild-type parental cells was 1000-fold less sensitive to camptothecin. The decreased expression of topoisomerase I in this cell line was compensated for by a corresponding increase in topoisomerase II expression. Postulated mechanisms responsible for the decreased expression of topoisomerase I include chromosomal deletions or by permethylation of the topoisomerase I gene. See, e.g., Tan, K. B., et al., Nonproductive rearrangement of DNA topoisomerase I and II genes: correlation with resistance to topoisomerase inhibitors. J. Natl. Cancer Inst. 81:1732 (1994).

The role for the P-glycoprotein-associated multidrug resistance (MDR) phenotype in camptothecin resistance has not been clearly defined. In comparison studies, MDR-expressing sublines were nine-fold more resistant to Topotecan and two-fold more resistant to 9-aminocamptothecin than the parental wild-type cells. See, e.g., Chen, A. U., et al., Camptothecin overcomes MDR1-mediated resistance in human KB carcinoma cell lines. Cancer Res. 51:6039 (1991). No increase in resistance was observed for camptothecin or 10,11-methylenedioxycamptothecin. While other investigators have confirmed these observations, this degree of MDR-associated resistance is much less than the 200-fold change in sensitivity typically described for classic MDR substrates, such as doxorubicin or etoposide, in the same experiments. See, e.g., Mattern, M. R., et al., In vitro and in vivo effects of clinically important camptothecin analogues on multidrug-resistant cells. Oncology Res. 5:467 (1993). The relevance of these observations to the clinical setting requires further study.

Another potential mechanism for camptothecin resistance is decreased intracellular drug accumulation, which was observed in vitro. The biochemical processes responsible for these decreased intracellular drug levels have not been identified, and unfortunately, little is currently known about the mechanisms of camptothecin influx and efflux from cells. Finally, resistance to a camptothecin prodrug, such as irinotecan, may result from decreased intracellular production of the active metabolite SN-38 by the irinotecan converting-enzyme carboxylesterase. A preliminary association has been reported between the measured converting-enzyme activity in different tumor cell lines and their relative sensitivity to irinotecan. See, e.g., Chen, S. F., et al., Human tumor carboxylesterase activity correlates with CPT-11 cytotoxicity in vitro. Proc. Am. Cancer Res. 35:365 (1994).

The key biochemical or molecular determinants of tumor response to clinical camptothecin therapy have not yet been identified. Because of the complex, stepwise pattern of drug-induced perturbations in cellular metabolism, it is possible that no single parameter will completely identify sensitive or resistant tumors. While the overall levels of topoisomerase I are important, other factors are also essential, including the degree of drug sensitivity of the topoisomerase I enzyme, the number of cleavable complexes stabilized, and the extent of ongoing DNA synthesis. Equally important, but even less understood, events that contribute to camptothecin cytotoxicity include DNA damage repair, the triggering of apoptosis, and alteration of the integrity of cell cycle control by, for example, G₂ check-point arrest. A detailed understanding of the relationship between each of these processes and camptothecin-induced cell death remains an important research goal.

F. Clinical Limitations of Currently-Available Camptothecin Analogues

An important limitation of currently-available camptothecins (CPTs), which contain the native 20(S) lactone E-ring, is that the lactone species (i.e., the biologically active moiety) persists in low concentrations (≦20%) of the total drug concentration) in human plasma at physiologic pH. It is well recognized that the lactone form of CPTs demonstrates substantially greater antitumor activity relative to the carboxylate form. The reported concentrations of the CPT lactone species are substantially higher in mice than in humans for CPT-11 and SN-38 (the active metabolite of CPT-11), 9-amino-camptothecin (9-NH₂—CPT, or 9-AC), 9-nitro-camptothecin (9-NO₂—CPT), and CPT. The low proportion and concentration of the lactone species of the CPTs in human plasma is thought to have a substantial effect in reducing the antitumor activity of CPTs containing the 20(S) lactone E-ring moiety.

In addition, the clinical utility of commercially available water-soluble CPTs may be limited by the following: reduced tissue diffusion and uptake, unfavorable variability in drug activation and/or metabolism, common clinical toxicities that can be dose-limiting, and susceptibility to tumor-mediated drug resistance mechanisms.

### VI. Karenitecin

Highly lipophilic camptothecin derivatives (HLCDs), particularly those containing silicon-based moieties, are effective anti-cancer drugs. One of the most noted of the silicon-containing HLCDs is Karenitecin (also known as BNP1350; cositecan; IUPAC Nomenclature: (4S)-4-ethyl-4-hydroxy-11-[2-(trimethylsilyl)ethyl]-1H-pyrano[3′:4′:6,7]indolizino[1,2-b]quinoline-3,14(4H,12H)-dione, and also referred to as 7-(2′-trimethylsilyl)ethyl camptothecin)). Karenitecin has been tested in human clinical trials, ranging from Phase I to Phase III, in the United States and internationally. U.S. Pat. Nos. 5,910,491; 6,194,579; 7,687,487; 7,687,496; and 7,687,497; and U.S. patent application Ser. No. 13/068,244, filed May 6, 2011; Ser. No. 13/573,294, filed Sep. 7, 2012; and Ser. No. 13/694,255, filed Nov. 13, 2012, which are all incorporated by reference herein in their entirety, describe certain compositions, formulations, and processes for synthesizing Karenitecin and other related HLCDs.

The molecular structure of Karenitecin is shown in (A), below).

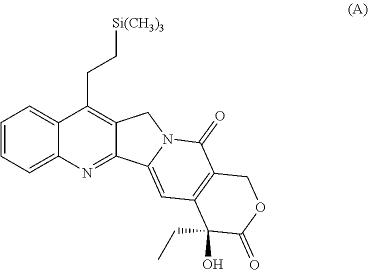

Karenitecin, and various analogs thereof, represent a novel class of cancer treating agents that have exhibited potent antineoplastic activity against common types of cancer including but not limited to cancers of the lung, breast, prostate, pancreas, head and neck, ovary, colon, as well as melanoma. While Karenitecin possesses Topoisomerase I inhibitory activity similar to that of other camptothecin derivatives, it also possess novel structural modifications that are rationally designed for superior bioavailability and tissue penetration, while concomitantly avoiding untoward metabolism and drug resistance mechanisms which are common in human and other mammalian cancers.

It may be ascertained from pharmacological and biochemical data, that many of the previously synthesized camptothecin analogs possess a number of inherent limitations which markedly decreases their usefulness as anti-cancer agents. In contrast, Karenitecin is a HLCD characterized by substantial lactone stability and long plasma half-life. In vitro studies conducted on a panel of over twenty (20) human cancer cell lines indicate that Karenitecin is a significantly more potent antitumor agent than either Topotecan or SN-38, the active metabolite of Irinotecan. Equilibrium dialysis studies with human plasma demonstrated that Karenitecin is 98 to 99% protein-bound. The free drug concentration in blood plasma is generally considered to be the pharmacologically active form in clinical pharmacology.

In addition, Karenitecin has significant utility as a highly efficacious cancer treating agent, and is significantly less toxic than previously disclosed camptothecin derivatives. Karenitecin also does not undergo A-ring or B-ring glucuronidation (and implicitly deglucuronidation). The lack of glucuronidation decreases deleterious physiological side-effects (e.g., diarrhea, leukopenia) and may also mitigate substantial intersubject variability in drug levels of the free metabolite and its glucuronide conjugate. Furthermore, Karenitecin is not a prodrug, thus it requires no metabolic activation.

Thus, in summation, Karenitecin: (i) possesses potent antitumor activity (i.e., in nanomolar or sub-nanomolar concentrations) for inhibiting the growth of human and animal tumor cells in vitro; (ii) is a potent inhibitor of Topoisomerase I; (iii) lacks susceptibility to MDR/MRP drug resistance; (iv) requires no metabolic drug activation; (v) lacks glucuronidation of the A-ring or B-ring; (vi) reduces drug-binding affinity to plasma proteins; (vii) maintains lactone stability; (viii) maintains drug potency; and (ix) possesses a low molecular weight (e.g., MW<600).

VII. Summary of Non-Clinical and Clinical Data with Karenitecin

Karenitecin is a novel, HLCD and is distinguished from other camptothecins on the basis of its highly novel chemical structure, possessing a tri-methyl silicon moiety. As previously discussed, some of the novel characteristics displayed by Karenitecin include, but are not limited to: (i) possesses potent antitumor activity (i.e., in nanomolar or sub-nanomolar concentrations) for inhibiting the growth of human and animal tumor cells in vitro; (ii) is a potent inhibitor of Topoisomerase I; (iii) lacks susceptibility to MDR/MRP drug resistance; (iv) requires no metabolic drug activation; (v) lacks glucuronidation of the A-ring or B-ring (which reduces inter-subject variability and gastrointestinal toxicity); (vi) reduces drug-binding affinity to plasma proteins; (vii) maintains lactone stability; (viii) maintains drug potency; and (ix) possesses a low molecular weight (e.g., MW<600). See, e.g., Yao, S., et al., Studies of the protein binding properties of Karenitecin (BNP1350), a novel highly lipophilic camptothecin analogue. AACR Abstract 1786 (2003); Hausheer, F. H., et al., Karenitecins: new preclinical developments with BNP1350; a novel, potent highly lipophilic camptothecin. AACR Abstract 741 (1999).

Karenitecin has a longer half-life in plasma, when compared with reported half-lives of various other commercially-available camptothecins. Karenitecin also appears to be insensitive to all commonly-known tumor-mediated drug resistance mechanisms, including Breast Cancer Resistance Protein (BCRP), which is recognized to be a tumor-mediated drug resistance factor in human cancer for camptothecins. See, e.g., Maliepaard, M., et al., Circumvention of breast cancer resistance protein (BCRP)-mediated resistance to camptothecins in vitro using non-substrate drugs or the BCRP inhibitor GF120918. Clin. Cancer Res. 7:935-941 (2001).

Karenitecin has demonstrated significant anti-tumor activity in vitro and in vivo against various human xenograft tumor models for various tumor types including, but not limited to, human central nervous system (CNS), colon, melanoma, lung, breast, ovarian carcinoma, and glioblastoma multiform. See, e.g., Van Hattum, A. H., et al., Novel camptothecin derivative BNP1350 in experimental human ovarian cancer: determination of efficacy and possible mechanisms of resistance. Int. J. Cancer. 100:22-29 (2002); Keir, S. T., Hausheer, F. H., et al., Therapeutic activity of 7-[(2-trimethylsilyl)ethyl)]-20(s)-camptothecin against central nervous system tumor-derived xenografts in athymic mice. Cancer Chemother. Pharmacol. 48:83-87 (2001); Van Hattum, A. H., et al., New highly lipophilic camptothecin BNP1350 is an effective drug in experimental human cancer. Int. J. Cancer 88:260-266 (2000); Hausheer, F. H., et al., Karenitecins: further developments with BNP1350: a novel, highly lipophilic, lactone stable camptothecin [abstract]. AACR Abstract 1360 (2000).

The relative antitumor activity of Karenitecin in preclinical models is similar or superior to the antitumor activity observed with other camptothecins, and Karenitecin has demonstrated a high degree (e.g., approximately 85%) of lactone stability in humans.

Preclinical toxicology studies of Karenitecin administered intravenously as a single daily dose for five consecutive days in Fischer rats (bolus) and beagle dogs (one-hour infusion) demonstrated reversible myelosuppression (predominantly neutropenia), diarrhea, emesis and mucositis (canine) and mild to moderate, reversible (one-hour duration) hypersensitivity histamine release-related reactions (canine) A preclinical toxicology study of various doses of Karenitecin administered orally as single daily doses for five consecutive days in beagle dogs demonstrated toxicities including: anorexia; weight loss; gastrointestinal toxicity (manifested as diarrhea with hemorrhage); occasional vomiting; and myelosuppression (evidenced by neutropenia, thrombocytopenia, lymphopenia, and transient decreases in erythrocyte numbers). The Maximum Tolerated Dose (MTD) of orally administered Karenitecin was 0.075 mg/kg; approximately 2-times that of intravenous (i.v.) Karenitecin. In addition, a toxicology study of various single daily doses of Karenitecin administered orally and intravenously for five consecutive days showed good tolerability of oral doses. Toxicities were reversible and included: anorexia (seen in i.v. groups, but not oral groups); weight loss (high in i.v. groups, negligible in oral groups); infusional toxicities; gastrointestinal toxicities (diarrhea and vomiting); and myelosuppression (neutropenia, thrombocytopenia, and lymphopenia). Gastrointestinal toxicities were dose-dependent, and were more severe in the higher drug treatment groups. Oral gastrointestinal toxicities were delayed and mild compared with i.v.-associated toxicities. Other clinical observations included: infusional toxicities, excitement, hyperpnea, facial and pinnae edema, pruritis, forced bowel movements, vomiting, increased tearing, and ptyalism.

To date, there have been no hypersensitivity reactions reported by subjects receiving Karenitecin. The dose-limiting toxicities (DLTs) of Karenitecin in humans, as determined in initial Phase I clinical studies, are reversible and non-cumulative neutropenia and thrombocytopenia.

Intravenous administration of Karenitecin has been evaluated in three Phase I clinical studies in subjects with the following cancer types: (i) advanced solid tumors (adult subjects) (see, e.g., Schilsky, R. L., Hausheer, F. H., et al., Phase I trial of karenitecin administered intravenously daily for five consecutive days in subjects with advanced solid tumors using accelerated dose titration [abstract]. ASCO Abstract 758 (2000)); (ii) refractory or recurrent solid tumors (pediatric subjects); and (iii) recurrent malignant glioma (adult subjects). Intravenous administration of Karenitecin has also been evaluated in four Phase 2 clinical studies in adult subjects with the following cancer types: (i) primary malignant glioma; (ii) third-line treatment of persistent or recurrent epithelial ovarian or primary peritoneal carcinoma; (iii) malignant melanoma (see, e.g., Hausheer, F. H., et al., Phase II trial of Karenitecin (BNP1350) in malignant melanoma: clinical and translational study [abstract]. ASCO Abstract 7554 (2004); and (iv) relapsed or refractory non-small cell lung cancer (see, e.g., Miller, A. A., et al., MR for the Cancer and Leukemia Group B. Phase II trial of karenitecin in subjects with relapsed or refractory non-small cell lung cancer (CALGB 30004) Lung Cancer 48:399-407 (2005)).

### VIII. Oral Administration of Karenitecin

A Phase I clinical trial (the “Oral Karenitecin Phase I Trial”) was performed to determine the maximum tolerated dose (MTD) of oral Karenitecin in subjects with solid tumors given in a dose-escalated manner (starting at 0.5 mg/m²) and administered 3-times per week (MWF or TTS) for 3 consecutive weeks followed by a one-week treatment rest. The objectives of the Oral Karenitecin Phase I Trial are described below.

Stage I Objectives
To characterize and compare the pharmacokinetics of oral and intravenous Karenitecin.
To assess the safety profile of oral Karenitecin given in a dose escalated manner (starting at 0.5 mg/m²) administered 3-times per week for three consecutive weeks followed by one-week rest (one treatment cycle) using the protocol-defined treatment schedule.

Stage I Study Design

In Stage I, subjects received Karenitecin as a single oral dose 3 times per week for 3 consecutive weeks followed by a one-week treatment rest. In Stage 1, some of subjects underwent pharmacokinetic sampling, and first received a single i.v. dose of Karenitecin (at the appropriate dosage level) followed by oral Karenitecin 4 to 7 days following the i.v. dose on a MWF or TTS schedule. Patients then received oral Karenitecin for all subsequent treatments. Patients who did not undergo pharmacokinetic sampling, only received oral Karenitecin on a MWF or TTS schedule.

Table II below, illustrates the dose escalations (or de-escalations) utilized in Stage 1 using an accelerated dose titration study design.

TABLE 2
Dose Escalation Schedule for Patients Participating in Stage I

| | Daily Dose of Oral Karenitecin | Dose of IV Karenitecin |
|---|---|---|
| | (mg/m²/day) | (mg/m²) (Day 7 only) |
| | (% increase/decrease | (% increase/decrease |
| Dose Level | from previous dose level) | from previous dose level) |
| −1 | 0.25 | 0.25 |
| 0 (starting | 0.5 (−50%) | 0.5 (−50%) |
| dose) | | |
| 1 | 1.0 (100%) | 1.0 (100%) |
| 2 | 1.5 (50%) | 1.5 (50%) |
| 3 | 2.0 (33%) | 2.0 (33%) |
| 4 | 2.5 (25%) | 2.5 (25%) |
| N | Previous dose level + | Previous dose level + |
| | 0.5 mg/m²/day | 0.5 mg/m²/day |

i.v. doses should be rounded to the nearest 0.1 mg (≦0.04 round down, ≧0.05 round up).
Oral doses should be rounded to the nearest 0.25 mg daily dose.

Stage 2 Objectives
To determine the MTD of orally administered karenitecin as a single daily dose, for 5 consecutive days (Monday through Friday) for 3 consecutive weeks followed by a one-week treatment rest.
To assess the pharmacokinetics of oral karenitecin.
To assess the safety profile of oral Karenitecin given in a dose escalated manner (starting at 0.5 mg/m²) using a protocol-defined treatment schedule.

Stage 2 Study Design

In Stage 2, Karenitecin was administered in a dose-escalated manner to approximately 16 subjects with solid tumors in order to determine the maximum tolerated dose (MTD). The initial dose escalation involved cohorts of one new subject per dose level. The starting dose was 0.5 mg/m² and dose escalation proceeded in 0.5 mg/m² increments. One-subject cohorts were utilized until the occurrence of a dose-limiting toxicity (DLT) or NCI CTCAE≧grade 2 toxicity was observed during the DLT observation period. Patients in Stage 2 received oral Karenitecin at the appropriate dose level on a MTWTF schedule for three (3) consecutive weeks, followed by a one-week rest period.

Summary of Oral Karenitecin Phase I Trial Results

In the Phase I clinical trial of orally administered Karenitecin (the “Oral Karenitecin Phase I Trial”), the oral administration of Karenitecin was generally well-tolerated. Additionally, in the Oral Karenitecin Phase I Trial, no progressive disease was observed for several months in heavily pre-treated patients with advanced forms of cancer who had failed prior therapies. On average, subjects enrolled in the Oral Karenitecin Phase I Trial had received approximately three (3) prior chemotherapy agent regimens, where often such regimens were comprised of more than one or two agents. Because this was a Phase I dose-finding study, the primary goal was to find the maximum tolerated dose and many subjects enrolled are usually not expected, in general, to remain on the study for long durations without evidence of disease progression.

A patient with advanced ovarian cancer participating in the Phase I trial of orally administered Karenitecin remained on the trial for approximately 16 months with no evidence of progressive disease. A patient with neuroendocrine cancer participating in the oral Karenitecin trial remained on the trial for approximately 23 months with no evidence of progressive disease. A patient with breast cancer participating in the oral Karenitecin trial remained on the trial for approximately 19.5 months with no evidence of progressive disease. A patient with non-small cell lung cancer participating in the oral Karenitecin trial remained on the trial for approximately 36 months with no evidence of progressive disease. A patient with pancreatic cancer participating in the oral Karenitecin trial remained on the trial for approximately 7 months with no evidence of progressive disease. A patient with melanoma participating in the oral Karenitecin trial remained on the trial for approximately 15 months with no evidence of progressive disease. A patient with peritoneal papillary adenocarcinoma cancer participating in the oral Karenitecin trial remained on the trial for approximately 8 months with no evidence of progressive disease. A patient with leiomyosarcoma participating in the oral Karenitecin trial remained on the trial for approximately 7 months with no evidence of progressive disease. A patient with refractory small cell cancer participating in the oral Karenitecin trial remained on the trial for approximately 6 months with no evidence of progressive disease. A patient with colorectal cancer participating in the oral Karenitecin trial remained on the trial for approximately 8 months with no evidence of progressive disease. A patient with prostate cancer participating in the oral Karenitecin trial remained on the trial for approximately 7 months with no evidence of progressive disease. A patient with sarcoma participating in the oral Karenitecin trial remained on the trial for approximately 9 months with no evidence of progressive disease. A patient with carcinoma of the parotid gland participating in the oral Karenitecin trial remained on the trial for approximately 12 months with no evidence of progressive disease.

### IX. Extending the Platinum-Free Interval Using Camptothecin Derivatives

The natural progression of ovarian cancer is typically characterized by the subsequent development of broad cross-resistance to various cancer treating agents. This resistance may develop from changes in drug-host metabolism, from the expansion of tumor cells to sites that are poorly responsive to cancer treating agents, from changes in expression levels of certain genes, and/or from biochemical changes at the cellular or subcellular level. The availability of novel non-cross-resistant cancer treating agents, such as Karenitecin, allows clinicians the opportunity to extend the platinum free interval at the time of first relapse and increase the likelihood of response to platinum re-induction at the second relapse, and even overcome or delay acquired platinum resistance in some subjects. The rationale for using a camptothecin derivative such as Karenitecin includes its novel mechanism of action, its comparable efficacy compared with paclitaxel in subjects with recurrent drug-sensitive disease, and its non-cumulative, well-characterized toxicity profile based upon clinical trial experience, including a recent Phase III clinical trial.

As fully discussed in Section V, supra, camptothecins such as Karenitecin have a novel mechanism of action distinct from platinum compounds, taxanes, alkylating agents, and various other cancer treating agents previously used in the treatment of ovarian cancer. Topoisomerase I is an enzyme that is critical for cell growth and proliferation. It catalyzes the cutting and ligating of a single DNA strand and is required for DNA replication, DNA repair, and gene expression. Karenitecin exerts its cytotoxic effect by disrupting this DNA breakage/religation process that occurs during replication, thus resulting in tumor cell death. The general mechanisms of antineoplastic drug resistance include decreased drug accumulation, altered drug metabolism, altered drug targets, and enhanced DNA repair capacity. Interestingly, Topoisomerases may be the final common cytotoxic pathway for several different classes of cancer treating agents (including platinum compounds). Drugs that act through topoisomerases prevent the re-ligation of DNA as a consequence of cleavable complex formation. Through this mechanism, Karenitecin can potentially interfere with repair from cisplatin-induced DNA damage (a major mechanism of cisplatin resistance).

Efficacy in Relation to Platinum Sensitivity

Upon relapse of disease, second-line therapy with a camptothecin derivative, such as Karenitecin, rather than the re-administration of platinum, could theoretically maximize the effectiveness of both agents. In various clinical trials, responses to the camptothecin derivative, topotecan, are highest when used in first-relapse, drug-sensitive disease; as well as responses to platinum therapy are highest when the platinum-free interval is extended. Conversely, second-line therapy with platinum prior to topotecan can actually promote the development of drug resistance and reduce the subsequent likelihood of response to topotecan.

Non-Cumulative Toxicity Profile

In addition to the overall cytotoxicity efficacy, a number of safety-related factors must be considered when selecting a second-line regimen in relapsed ovarian cancer, including quality of life, the type and degree of prior platinum- and taxane-induced toxicities, and subject preferences about their treatment. Guidelines developed at a consensus conference of the National Institutes of Health (NIH) state that the goals of follow-up and treatment of relapsed ovarian cancer need to incorporate quality-of-life considerations as an integral part of treatment. See, e.g., NIH Consensus Development Panel on Ovarian Cancer. Ovarian cancer: screening, treatment, and follow-up. JAMA 273:491-497 (1995). In addition to various cumulative hematological toxicities, approximately one-third of subjects treated with platinum/taxane doublet regimens still have persistent neuropathy at the time of relapse. Patients with cumulative peripheral neuropathy may suffer a significant loss of manual dexterity, balance, and coordination that interferes with daily activities and markedly reduces quality of life. It is important to avoid potentially neuropathic agents in this population. As described earlier, Karenitecin has a predictable, manageable, and non-cumulative side effect profile and is unlikely to add to the cumulative non-hematologic toxicities caused by prior therapy, including peripheral neuropathy and renal toxicity. Retreatment with platinum compounds in relapsed ovarian cancer may be compromised by cumulative toxicities, especially in heavily pretreated subjects, older subjects, and subjects with preexisting nephrotoxicity. Administration of cancer treating agents with substantial toxicity to subjects with multiple prior therapies may require dose delays or dose reductions because of, e.g., diminished bone marrow reserves (from carboplatin) or renal impairment (from cisplatin). Earlier utilization of Karenitecin may reduce the likelihood of dose-limiting hematologic toxicities.

In sum, despite the high overall clinical response rates achieved with combination platinum-taxane therapy (up to 80%), including a high proportion of complete responses, most subjects subsequently relapse and develop drug-resistant disease. Therefore, the primary goal of therapy in relapsed ovarian cancer is to extend survival by maximizing all available therapies while minimizing deleterious side effects and preserving quality of life. See, e.g., NIH Consensus Development Panel on Ovarian Cancer. Ovarian cancer: screening, treatment, and follow-up. JAMA 273:491-497 (1995). The choice of therapy for relapsed ovarian cancer is generally based upon the timing and characteristics of the relapse, as well as the type and extent of the prior treatment. Current guidelines recognize the importance of platinum sensitivity with regard to prognosis and likelihood of response to retreatment. See, Id. Further prolongation of the platinum free interval through early use of a camptothecin derivative, such as Karenitecin, can maximize the therapeutic utility of each drug while enabling clinicians to better manage individual toxicity profiles, thereby optimizing quality of life.

### X. Summary of BNP1350, SN28, and Topotecan Testing in Mucinous Cell Lines

In vitro studies were conducted in human mucinous ovarian cancer cell lines including JHOM2B, OMC3, and COV644. In one series of experiments, the dose response curves for BNP1350 (also known as Karenitecin), SN-38 (Irinotecan), and Topotecan were obtained and IC50 values were determined. In a second series of experiments, the effect of BNP7787 (2,2′-dithio-bis-ethane sulfonate; Tavocept) on BNP1350-induced cytotoxicity at selected BNP1350 concentrations was examined.

In brief, Tavocept is a sulfur-containing, amino acid-specific, small molecule that possesses the ability to function as a multi-target modifier and/or modulator of the function of multiple target molecules of therapeutic interest. Tavocept mediates the non-enzymatic xenobiotic modification of sulfur-containing amino acid residues (e.g., cysteine) on proteins. As an engineered, non-naturally occurring agent (i.e., xenobiotic), Tavocept is autocatalytic and requires no protein cofactor to cause the xenobiotic modification of cysteine, but appears to be specific for cysteine residues located within a particular structural context (i.e., not all cysteines in a protein are so modified). Tavocept-mediated, xenobiotic modification represents a novel mechanism of action for a cancer treating agent and can be compared, to a degree, with post-translational modifications of cysteine residues in proteins (see, Table 3, below). By way of non-limiting example, some of the important elements of Tavocept's effectiveness as a compound in the treatment of cancer are its selectivity for normal cells versus cancer cells and its absence of interference with the anti-cancer activity of cancer treating agents. In vitro studies demonstrated that Tavocept does not interfere with paclitaxel induced apoptosis, as assessed by PARP cleavage, Bcl-2 phosphorylation, and DNA laddering in human breast, ovarian and lymphoma cancer cell lines. Additionally, Tavocept was shown not to interfere with paclitaxel- and platinum-induced cytotoxicity in human cancer cell lines.

The believed mechanisms underlying the absence of interference with anti-cancer activity by Tavocept are multifactorial and, as previously discussed, may involve its selectivity for normal cells versus cancer cells, inherent chemical properties that have minimal impact in normal cells on critical plasma and cellular thiol-disulfide balances, and its interactions with cellular oxidoreductases, which are key in the cellular oxidative/reduction (redox) maintenance systems.

In addition to the absence of interference with anti-cancer activity, results from in vivo studies have shown that Tavocept may elicit the restoration of apoptotic sensitivity in tumor cells through, e.g., thioredoxin- and glutaredoxin-mediated mechanisms and this may be an important element of its effectiveness as a cancer treating agent.

TABLE 3
Tavocept Cysteine-Specific Protein Modifications

| | | Protein |
|---|---|---|
| | | Cofactor(s) |
| Modification | Specificity | Required? |
| Tavocept-mediated | Cysteines near or in α-helices, | No |
| xenobiotic | with nearby residues to accept the | |
| modification | cysteine thiol proton and | |
| | stabilize the cysteinyl thiolate | |
| Glutathionylation | May involve cysteines with altered | Can be |
| | pKa's (vicinal to lysine, | autocatalytic |
| | arginine or histidine) | or protein |
| | | catalyzed |
| Nitrosylation | Possible specificity at the tertiary | No |
| | environment level around cysteine | |
| Prenylation | Varied sequences around target | Yes |
| (Farnesylation, | cysteine with a CaaX motif (a = | |
| geranylgeranylation) | aliphatic amino acid; X = one | |
| | of several amino acids depending | |
| | on protein) | |
| Palmitoylation | Varied Sequences | Can be |
| | | autocatalytic |
| | | or protein |
| | | catalyzed |

In the studies conducted in human mucinous ovarian cancer cell lines, the percent of cell survival was determined using the SRB assay relative to survival seen in cells that were not treated with any test article.
For dose/response curves and IC₅₀ determinations, cells were exposed to increasing drug concentrations as set forth below:
   BNP1350 (0.0156-2 μM)
   SN-38 (0.0156-2 μM)
   Topotecan (1.56-20 μM)
   No drug control (DMSO solvent only) were included and did not have any effect on cell growth for any cell line.
For effects of BNP7787 on BNP1350-induced cytotoxicity, cells were treated with:
   BNP7787 at 1 and 10 mM with approximate IC₂₅ values of BNP1350 for each cell line.
   BNP7787 at 1 mM and 10 mM with approximate IC₅₀ values of BNP1350 for each cell line.
   Controls consisting of no drug treatment, BNP7787 as a single agent, and BNP1350 as a single agent were included.

### A. Materials and Methods

The JHOM2B and OMC3 cell lines were purchased from Riken Cell Bank (Japan). The COV644 cell line was purchased from SigmaAldrich. Cells were maintained as monolayered cultures in T-75 flasks and then seeded to microtiter plate wells for experiments. Population doubling-times for these three cell lines were long (i.e., exceeding 40 hours, and for some of the cell lines approaching 96 hours); therefore, the Applicants conducted initial pilot experiments and determined optimal seeding density to obtain A₅₇₀ values in untreated controls that would fall within the linear range of the SRB assay after seven (7) total days of cell growth. JHOM2B cells were cultured in DMEM:F12 (1:1)+0.1 mM non-essential amino acids (NEAA)+10% FBS and were seeded at 6,000 cells per well. OMC3 cells were cultured in HamF12+10% FBS and were seeded at 8,000 cells per well. COV644 cells were cultured in DMEM+2 mM glutamine+10% FBS and were seeded at 6,000 cells per well.

The cells were manipulated in a Microzone laminar flow hood traditionally used for maintaining aseptic environments around automated instrumentation such as microplate robots, and the like. Cells were grown and maintained at 37° C. in a humidified atmosphere containing 5% CO₂ in a water jacketed cell culture incubator (Forma Scientific). Cells were counted using a ViCell counter (Beckman-Coulter). An automated plate washer/stacker (ELx405/BioStak from BioTek Instruments) and a microplate reader (SpectraMax Plus from Molecular Devices) were used for conducting the SRB assay and determining A₅₇₀ values for wells in the plates and the percentage of control values. Prior to SRB assays, cell viability was monitored by evaluation of microtiter plate wells. Dead cells detach and float while living cells remain attached to the bottom of the cell well.

### B. Test Articles

SN-38 (Irinotecan; Sigma H0165, lot MKBQ4981) and Topotecan (Sigma T2705, lot 052M4728V) were purchased from SigmaAldrich. BNP1350 (lot KN-C192-135) and BNP7787 (lot 205001) were synthesized at BioNumerik Pharmaceuticals, Inc. (San Antonio, Tex.). BNP1350, SN38, and Topotecan were prepared as 2.5, 2.5, and 25 mM stocks in DMSO (ATCC, cell culture grade).

### C. Assay Approach

The sulforhodamine B (SRB) cytotoxicity assay was used to assess cell survival. Briefly, after the medium was decanted from individual plate wells, trichloroacetic acid (100 μL of 10.0 percent solution) was added to each well, and the plates were incubated at 4° C. for 1 hour. The plates were washed five times with water using an automated microplate washer (Model ELx405, Bio-Tek Instruments), SRB solution (100 μL of 0.4% SRB dissolved in 1.0 percent acetic acid) was added, and plates remained at room temperature for 15 minutes. The plates were then washed five times using acetic acid (1.0 percent), air dried, and bound dye was solubilized in Tris base (150 μL, 10 mM). Plates were agitated (gently) for 5 minutes and the absorbance values of the SRB dye-protein adduct at 570 nanometers (A₅₇₀) were determined using an automated microtiter plate reader (SpectraMax Plus, Molecular Devices). IC₅₀ values were extrapolated from dose response curves of the data using OriginLab Software.

### Results

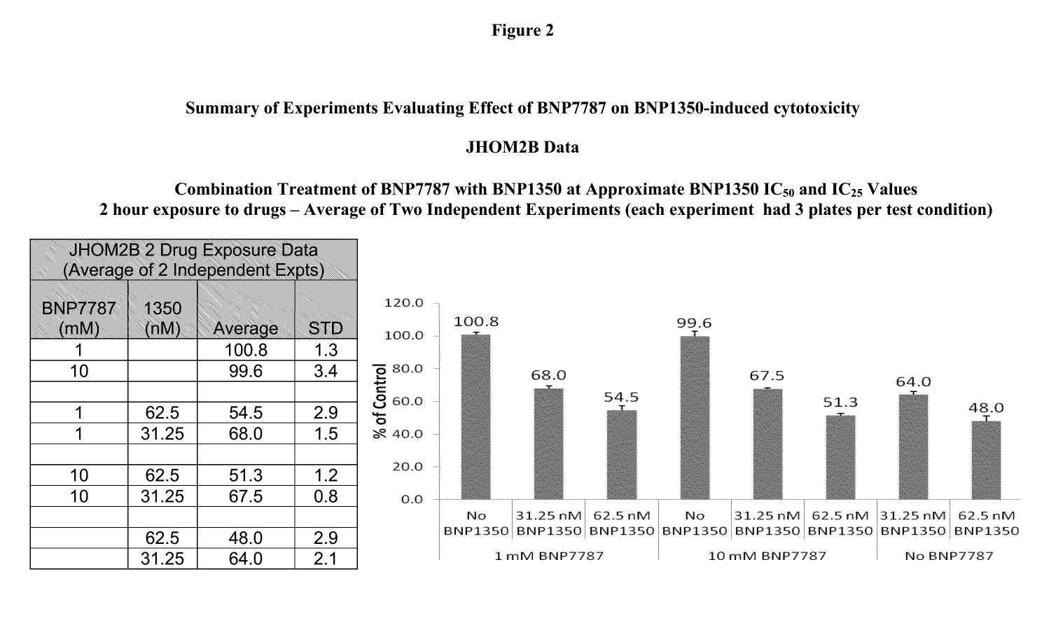

IC₅₀ Values Determined from Dose-Response Curves
IC₅₀ values determined from dose-response curves are summarized in the tables below.
Tables 4a and 4b illustrate IC₅₀ values determined from does-response results in tabular summaries for JHOM2B data. FIG. 2 illustrates a summary of experiments evaluating effect of BNP7787 on BNP1350-induced cytotoxicity for JHOM2B data.

TABLE 4a
JHOM2B IC₅₀ Tabular Summary
JHOM2B Cell Line
2 hour drug exposure - IC₅₀ micromolar

| | Test 1 (2 | Test 2 (3 | | Average |
|---|---|---|---|---|
| Compound | replicate plates) | replicate plates) | Average | STD |
| 1350 | 0.061 ± 0.004 | 0.078 ± 0.009 | 0.0695 | 0.012 |
| SN38 | 0.61 ± 0.13 | 0.723 ± 0.028 | 0.6665 | 0.080 |
| Topotecan | 4.75 ± 0.25 | 5.59 ± 0.28 | 5.17 | 0.590 |

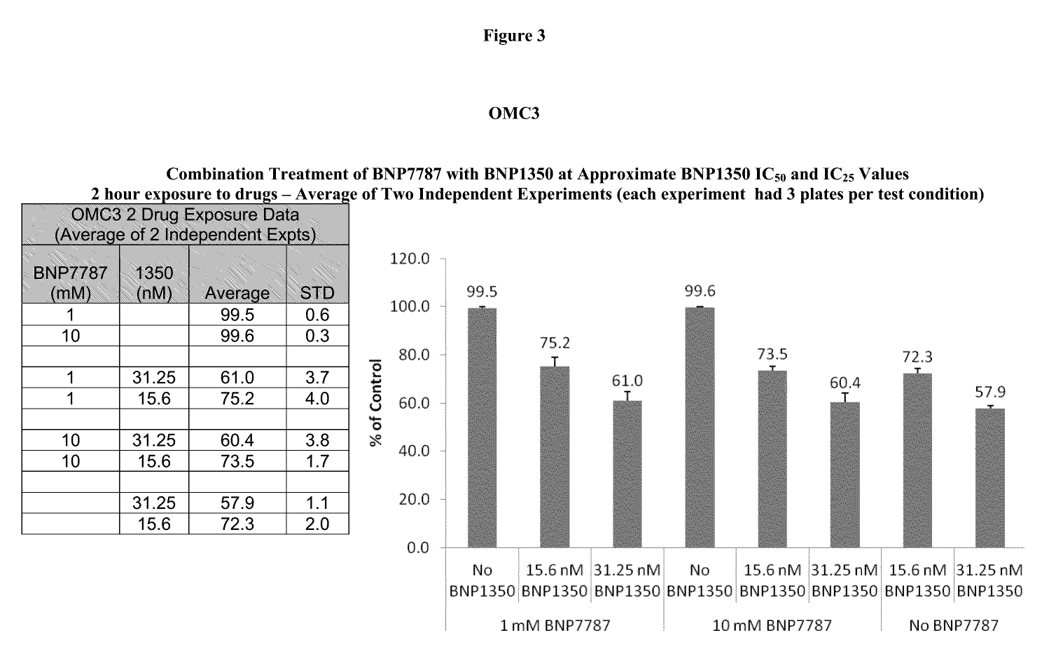

Tables 5a and 5b illustrate IC₅₀ values determined from dose-response results in tabular summaries for OMC3 data. FIG. 3 illustrates a summary of experiments evaluating effect of BNP7787 on BNP1350-induced cytotoxicity for OMC3 data.

TABLE 5a
OMC3 IC₅₀ Tabular Summary
OMC3 Cell Line
2 hour drug exposure - IC₅₀ micromolar

| | Test 1 (2 | Test 2 (3 | | Average |
|---|---|---|---|---|
| Compound | replicate plates) | replicate plates) | Average | STD |
| 1350 | 0.057 ± 0.004 | 0.042 ± 0.006 | 0.0495 | 0.011 |
| SN38 | 0.55 ± 0.06 | 0.58 ± 0.127 | 0.565 | 0.021 |
| Topotecan | 3.12 ± 0.18 | 4.76 ± 0.25 | 3.94 | 1.16 |

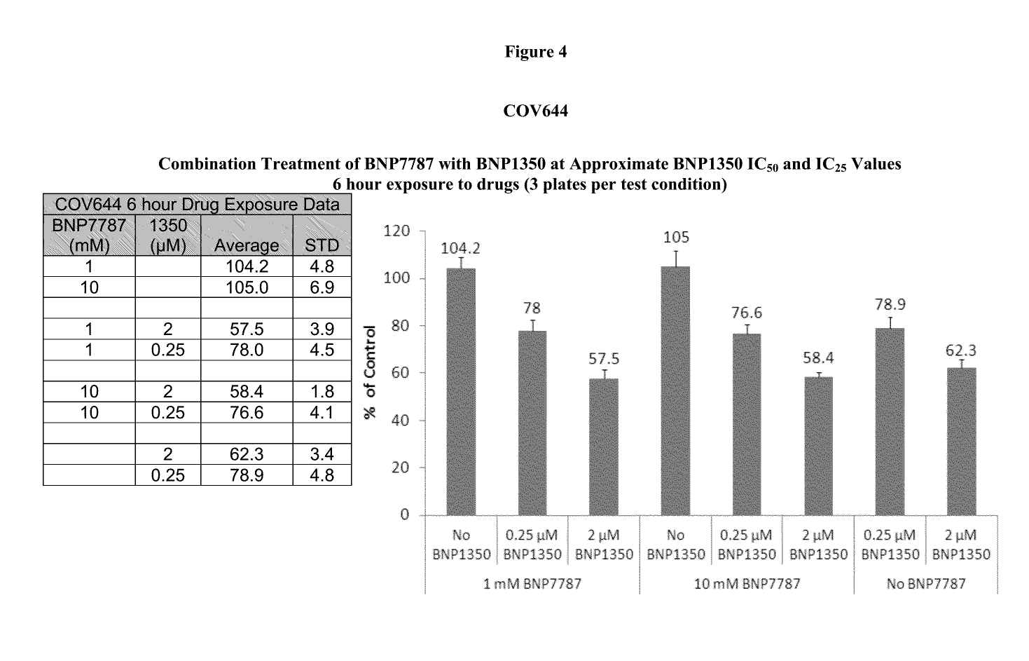

Tables 6a and 6b illustrate IC₅₀ values determined from does-response results in a tabular summaries for COV644 data. FIG. 4 illustrates a summary of experiments evaluating effect of BNP7787 on BNP1350-induced cytotoxicity for COV644 data.

Table 6a COV644 Attempts to Determine IC₅₀ Summary

The 1050 values exceeded the solubility in medium for each compound—the limit of solubility in cell culture medium for the compounds were as follows: 2 micromolar for BNP1350 and SN38, and 20 micromolar for Topotecan. Cells were treated for periods of 2.5 hours or 6 hours and, in each case, the 1050 values were not reached. Longer treatments were not performed.

TABLE 6a
COV644 Cell Line
2, 5, and 6 hour drug exposures IC₅₀ micromolar
(Treatments for did not reach 50% of Control in experiment variations)

| | Test 1 (2 | Test 2 (2 to 3 | | Average |
|---|---|---|---|---|
| Compound | replicate plates) | replicate plates) | Average | STD |
| 1350 | >2 | >2 | >2 | ND |
| SN38 | >2 | >2 | >2 | ND |
| Topotecan | >20 | >20 | >20 | ND |

*Note that data from 6 hour drug exposure was very similar to the data from the 5 hour drug exposures shown below.

### Major Conclusions From Above Cell Line Studies:

Dose response curves showed that BNP1350 (Karenitecin) was much more potent compared with SN38 and topotecan in this study for each of the mucinous cell lines (JHOM2B, OMC3, and COV644).
BNP1350 IC₅₀ values were: JHOM2B 69.5 nM; OMC3 49.5 nM; and COV644>2 μM.
SN38 IC₅₀ values were: JHOM2B 667 nM; OMC3 565 nM; and COV644>2 μM.
Topotecan IC₅₀ values were: JHOM2B 5.17 μM; OMC3 3.94 μM; and COV644>20 μM.
Combination treatment where BNP7787 (1 or 10 mM) was added simultaneously with approximate IC₅₀ and IC₂₅ values of BNP1350 for each cell lines indicated no notable effect on BNP1350-induced cytotoxicity.
BNP7787 alone (1 and 10 mM) did not affect cell survival.

### XI. Karenitecin Phase III Trial Design

Conducting the Karenitecin Phase III Trial disclosed in the instant patent application was critical to allow the evaluation of Karenitecin in this specific and rigorously selected subject population with a larger number of subjects in which detailed measurements of PFS, total number of subjects treated, safety related events, and other important clinical observations could be made in a larger subject population. Additionally, highly specific or more advanced methodologies were used to, e.g., initially select or diagnosis the subject population of the instant Phase III clinical trial. By way of non-limiting example, PFS was radiographically determined by an Independent Radiological Committee (IRC).

Prior to the instant Phase III clinical trial, the primary treatment goals in subjects with recurrent advanced ovarian cancer was improvement in quality of life and overall length of life; as these subjects were generally regarded as not curable. Chemotherapy was administered to these subjects as palliative treatment, and there is subjective evidence that cancer treating agents can improve quality of life in these subjects, but, as yet, there are no randomized studies performed which have compared cancer treating agents to best supportive care. Accordingly, a new treatment in this setting that has similar or better efficacy and/or more tolerability would be potentially highly beneficial for this subject population.

The Karenitecin Phase III Trial was a multi-center, multi-national, randomized, open-label, active-controlled, Phase III clinical study to evaluate the safety and efficacy of Karenitecin compared with Topotecan; wherein the drugs were administered to each trial subject as a single, daily intravenous dose of either Karenitecin or Topotecan—[Karenitecin 1.0 mg/m²/day×5 (first 5 consecutive days per cycle) in a 60 minute i.v. infusion or Topotecan 1.5 mg/m²/day×5 (first 5 days consecutive days per cycle) in a 30 minute i.v. infusion] every 21 days in subjects with stage III/IV advanced epithelial ovarian cancer who are resistant or refractory to platinum- and taxane-based cancer treating agent regimens, as indicated by relapse/progression while currently on, or within 6 months of completion of, platinum/taxane treatment in a first-line or second-line setting. In addition, subjects with a best response of stable disease (hereinafter “SD”) after a total of 6 cycles of platinum/taxane treatment in the first-line setting were be considered platinum-resistant.

All subjects admitted to the Karenitecin Phase III Trial were documented to be platinum- and/or taxane-resistant or refractory and have incurable disease. All subjects admitted to the clinical study must have had their disease progress while receiving cancer treating agent treatment or within 6 months of first or second line platinum- and/or taxane-based treatment. It is important to note that, currently, there is no FDA-approved cancer treating agent for this specific aforementioned indication.

Approximately 80 study centers participated in this clinical study. The primary endpoint was Progression Free Survival (PFS); defined as the time period from the date of randomization to the date of first radiographically documented progressive disease or disease progression (“PD”) or date of death due to any cause, taking the event date that occurs first.

Several cancer treating agents are approved for use in subjects who have failed initial treatment for advanced ovarian cancer. Most, if not all approved agents for the treatment of subjects with advanced ovarian cancer are associated with significant toxicity, and therefore new agents need to be developed to assist in the achievement of the treatment goals. Topotecan has been approved by the FDA for the treatment of metastatic carcinoma of the ovary after failure of initial or subsequent chemotherapy agent therapy. Topotecan has shown a trend to comparable or superior efficacy compared with both paclitaxel and Doxil in subjects with platinum-resistant or refractory ovarian cancer. See, e.g., Gordon A N, Fleagle J T, Guthrie D, Parkin D E, Gore M E, Lacave A J. Recurrent epithelial ovarian carcinoma: a randomized phase III study of pegylated liposomal doxorubicin versus Topotecan. J. Clin. Oncol. 19(14):3312-3322 (1991); Ten Bokkel Huinink W, Gore M, Carmichael J, et al. Topotecan versus paclitaxel for the treatment of recurrent epithelial ovarian cancer. J. Clin. Oncol. 15(6):2183-2193 (1997).

The hallmark toxicity of Topotecan is myelosuppression, which may also compound bone marrow toxicity from prior platinum therapy, thus necessitating very careful monitoring of hemoglobin (Hgb) levels, platelet counts, and neutrophil counts; as well as treatment interventions that include treatment delays, dose reductions, growth factor support, and RBC transfusions. See, e.g., Armstrong D K, Spriggs D, Levin J, Poulin R, Lane S. Hematologic safety and tolerability of Topotecan in recurrent ovarian cancer and small cell lung cancer: an integrated analysis. Oncologist. 10(9):686-694 (2005).

The Primary and Secondary Endpoints of the Karenitecin Phase III Trial disclosed and claimed herein were as follows:

### Primary Endpoint:

The primary endpoint was Progression Free Survival (PFS); defined as the time period from the date of randomization to the date of first radiographically documented progressive disease (PD) or date of death due to any cause, taking the event date that occurs first. The date of PD was determined by radiographically objective disease (RECIST) measurement.

### Secondary Endpoints:

Overall Survival (OS), defined as the time from the date of randomization to the date of death due to any cause.
Incidence of anemia, defined as the proportion of subjects who experience ≧grade 3 anemia based on National Cancer Institute Common Terminology Criteria for Adverse Events (NCI-CTCAE) criteria at any time post-baseline after receiving study treatment.
Incidence of neutropenia (including febrile neutropenia), defined as the proportion of subjects who experience ≧grade 3 neutropenia based on NCI-CTCAE criteria at any time post-baseline after receiving study treatment.
Incidence of thrombocytopenia, defined as the proportion of subjects who experience ≧grade 3 thrombocytopenia based on NCI-CTCAE criteria at any time post-baseline after receiving study treatment.

Patients underwent procedures throughout four (4) defined periods in this study, which are briefly described below. Various clinical, laboratory, and disease evaluations were required for each of these for each periods.

Period I (Screening and Randomization):

Patient eligibility was determined by compliance with protocol-specified inclusion and exclusion criteria. Patients who signed the informed consent, and successfully complete the screening process, including documentation of disease status by radiographic measures, were randomized to receive study treatment.

Period II (Active Treatment):

During this period, subjects received either Karenitecin (1.0 mg/m²/day administered as a 60-minute intravenous infusion) or Topotecan (1.5 mg/m²/day administered as a 30-minute intravenous infusion) daily for 5 consecutive days, every 21 days (one treatment cycle=21 days). Patients continued the study treatment until they meet one of the criteria listed below:
Patients with PD discontinued study treatment, and progressed to Period III.
Patients with SD or partial response (PR) could continue study treatment provided that they (a) continued to have evidence of clinical benefit (either objective tumor response or the absence of PD), and (b) they did not experience unacceptable treatment-related toxicity that was deemed by the treating physician to endanger the safety of the subject if they were to continue study treatment.
Patients who experienced a documented (radiographic) complete response (CR) at any time would continue treatment for 2 cycles (approximately 6 additional weeks) following the initial documentation of CR, provided that the subject did not experience any unacceptable treatment-related toxicity.

After completion of the additional 2 cycles (6 weeks) of treatment, subjects had repeat radiographic documentation of the extent of disease to confirm the CR. After confirmation (radiographic) of the CR, the subject has completed study treatment, and will progress to Period III.

Period III (End of Treatment):

Within ±3 days of date of treatment discontinuation, end-of treatment procedures were conducted during Period III. Tumor measurements and response assessments need to be completed during Period III if the regularly-scheduled tumor measurements/response assessments fall into the Period III time interval. Tumor measurements/response assessments must remain on the 6 week schedule. Radiographic scans, response assessments, and CA-125 levels will continue to be collected as described in Period IV.

Period IV (Follow-Up for Progression and Survival):

All subjects were followed for progression and survival. All subjects were assessed for best overall response at the time they reach PD or start any alternative therapy.

Follow-Up for Progression (Patients Discontinuing for Reasons Other than PD):
Patients who discontinue from the study for any reason other than PD must continue to undergo radiographic scans, response assessments, and CA-125 level assessments every 6 weeks (±5 days) until PD or until the initiation of new treatment, after which they will proceed to follow-up for survival.

Follow-Up for Survival (all Subjects):
Patients with documented PD will be followed up for survival (and date of any alternative therapy) by telephone and/or letter confirmation every 3 months until death.

### XII. Phase III Clinical Trials Results

Simultaneous statistical analyses were performed on the clinical trial data from the time points when 254 and 338 Progression Free Survival (PFS) events occurred during the aforementioned Karenitecin Phase III Trial and the following results were observed. Unless otherwise noted, reported data is based on all patient events available at the time of analysis.
A greater than 6 week median progression-free survival (PFS) advantage in favor of Karenitecin (as compared with Topotecan) in subjects having advanced epithelial ovarian cancer which exhibits evidence of being refractory or resistant to platinum/taxane-based cancer treating agent therapy (P-value=0.261; Hazard Ratio (HR)=0.885). Median PFS was approximately 24.3 weeks for the Karenitecin arm compared to 17.8 weeks for the Topotecan arm. The PFS for Karenitecin and Topotecan was determined using an Independent Radiologic Committee (IRC).
A 2 month median progression-free survival (PFS) advantage in favor of Karenitecin (as compared with Topotecan) was found in the “Histopathology class: Mucinous adenocarcinoma” subtype of trial subjects. The PFS benefit for Karenitecin in comparison to Topotecan was determined using an Independent Review Committee (IRC). Median PFS was approximately 17.8 weeks for the Karenitecin arm for this subtype compared to 9.1 weeks for the Topotecan arm for this subtype. In addition, an overall survival (OS) of 35 months was observed in the third quartile of the Karenitecin arm for the Mucinous adenocarcinoma ovarian cancer subtype as compared to an OS of 19.4 months for the third quartile of the Topotecan arm for this subtype. An improvement in the overall survival hazard ratio in favor of Karenitecin (as compared with Topotecan) was also observed in the “Histopathology class: Mucinous adenocarcinoma” subtype of trial subjects, resulting in an observed hazard ratio of 0.841, P-value 0.7321.
A median progression-free survival (PFS) of approximately 8.2 weeks in favor of Karenitecin (as compared with Topotecan) was found in the subpopulation of subjects who were either refractory or resistant to platinum- and/or taxane-based cancer treating agent therapy and/or had the mucinous adenocarcinoma sub-type of ovarian cancer (P-value=0.0849; HR=0.770 in the first 254 subjects). Median PFS was approximately 26.9 weeks for the Karenitecin arm for this subpopulation of patients compared to 18.7 weeks for the Topotecan arm for this subpopulation. The PFS benefit for Karenitecin in comparison to Topotecan was determined using an Independent Radiologic Committee (IRC).
A 3 month median progression-free survival (PFS) advantage in favor of Karenitecin (as compared with Topotecan) was found in the “Best response stable disease (SD) after 6 cycles in a first-line setting” sub-category of trial subjects (P-value=0.9908; HR=0.992). The PFS benefit for Karenitecin and Topotecan was determined using an Independent Radiologic Committee (IRC).
A 6.5 week median progression-free survival (PFS) advantage in favor of Karenitecin (as compared with Topotecan) was found in the “Ovary as primary site of disease” sub-category of trial subjects (P-value=0.2606; HR=0.885). The PFS benefit for Karenitecin in comparison to Topotecan was determined using an Independent Radiologic Committee (IRC).
A 4.2 month median progression-free survival (PFS) advantage in favor of Karenitecin (as compared with Topotecan) was found in the “FIGO Stage IIIB” sub-category of trial subjects (P-value=0.6225; HR=0.722). The PFS benefit for Karenitecin in comparison to Topotecan was determined using an Independent Radiologic Committee (IRC).
A 2.7 month median progression-free survival (PFS) advantage in favor of Karenitecin (as compared with Topotecan) was found in the “FIGO Stage IV” sub-category of trial subjects (P-value=0.0556; HR=0.741). The PFS benefit for Karenitecin in comparison to Topotecan was determined using an Independent Radiologic Committee (IRC). An improvement in the overall survival hazard ratio in favor of Karenitecin (as compared with Topotecan) was also observed in the “FIGO Stage IV” subtype of trial subjects, resulting in an observed hazard ratio of 0.892, P-value 0.4516.
A 2.8 month median progression-free survival (PFS) advantage in favor of Karenitecin (as compared with Topotecan) was found in the “FIGO Stage IV” sub-category of trial subjects, with the analysis of subjects based on the first 254 PFS events in the clinical trial (P-value=0.0260; HR=0.669). The PFS benefit for Karenitecin in comparison to Topotecan was determined using an Independent Radiologic Committee (IRC).
A 2.5 month median progression-free survival (PFS) advantage in favor of Karenitecin (as compared with Topotecan) was found in the “Histological Stage: G2-moderately differentiated” sub-category of trial subjects (P-value=0.2982; HR=0.835). The PFS benefit for Karenitecin in comparison to Topotecan was determined using an Independent Radiologic Committee (IRC).
A 1.6 month median progression-free survival (PFS) advantage in favor of Karenitecin (as compared with Topotecan) was found in the “Histopathology class: serous adenocarcinoma” sub-category of trial subjects (P-value=0.2829; HR=0.873). The PFS benefit for Karenitecin in comparison to Topotecan was determined using an Independent Radiologic Committee (IRC).
A 2.7 month median progression-free survival (PFS) advantage in favor of Karenitecin (as compared with Topotecan) was found in the “Histopathology class: Adenocarcinoma (grade ≧2) not otherwise specified” sub-category of trial subjects (P-value=0.3700; HR=0.684). The PFS benefit for Karenitecin in comparison to Topotecan was determined using an Independent Radiologic Committee (IRC). An improvement in the overall survival hazard ratio in favor of Karenitecin (as compared with Topotecan) was also observed in the “Histopathology class: Adenocarcinoma (grade ≧2) not otherwise specified” subtype of trial subjects, resulting in an observed hazard ratio of 0.898, P-value 0.7893.
A 3.9 month median progression-free survival (PFS) advantage in favor of Karenitecin (as compared with Topotecan) was found in the “ECOG Performance Status 0” sub-category of trial subjects (P-value=0.0249; HR=0.662). The PFS benefit for Karenitecin in comparison to Topotecan was determined using an Independent Radiologic Committee (IRC).
A 1.5 month median progression-free survival (PFS) advantage in favor of Karenitecin (as compared with Topotecan) was found in the “ECOG Performance Status 2” sub-category of trial subjects (P-value=0.7481; HR=0.896). The PFS benefit for Karenitecin in comparison to Topotecan was determined using an Independent Radiologic Committee (IRC).
Consistent with the overall trial population, the median progression-free survival (PFS) advantage in favor of Karenitecin (as compared with Topotecan) was also observed to improve for the resistant subject sub-population (P-value=0.144; HR=0.822).
A 1.7 week median progression-free survival (PFS) advantage in favor of Karenitecin (as compared with Topotecan) was found in the “Histological Stage: G1-well differentiated” sub-category of trial subjects (P-value=0.8561; HR=0.904). The PFS benefit for Karenitecin in comparison to Topotecan was determined using an Independent Radiologic Committee (IRC). An improvement in the overall survival hazard ratio in favor of Karenitecin (as compared with Topotecan) was also observed in the “Histological Stage: G1-well differentiated” subtype of trial subjects, resulting in an observed hazard ratio of 0.9674, P-value 0.9612.
An improvement in the overall survival hazard ratio in favor of Karenitecin (as compared with Topotecan) was also observed in the “Histopathology Class: Undifferentiated carcinoma” subtype of trial subjects, resulting in an observed hazard ratio of 0.701, P-value 0.6426.
An increase in the median number of treatment cycles able to be given to patients was observed for Karenitecin compared to Topotecan in the Karenitecin Phase III Trial, with a median of 6.0 treatment cycles for the Karenitecin arm of the Karenitecin Phase III Trial compared to a median of 5.0 treatment cycles for the Topotecan arm of the Karenitecin Phase III Trial.
The Karenitecin arm also demonstrated important safety/toxicity advantages with respect to the reduction of anemia (P-value=0.049) and thrombocytopenia (P-value=0.073). The number of grade 3 or 4 anemia events was reduced by 27.3% in the Karenitecin arm of the Karenitecin Phase III Trial in comparison to the Topotecan arm of the Karenitecin Phase III Trial. The number of grade 3 or 4 thrombocytopenia events was reduced by 37.4% in the Karenitecin arm of the Karenitecin Phase III Trial in comparison to the Topotecan arm of the Karenitecin Phase III Trial. A reduction in grade 4 neutropenia for the Karenitecin arm of the Karenitecin Phase III Trial was observed as well, with grade 4 neutropenia reduced by 38.2% in the Karenitecin arm of the Karenitecin Phase III Trial in comparison to the Topotecan arm of the Karenitecin Phase III Trial. In addition, no safety concerns related to Karenitecin were noted during the entire duration of the instant Phase III clinical study as monitored by the independent Data and Safety Monitoring Board (DSMB), and there were also no reports of reportable safety events related to Karenitecin which occurred to the Inventor's knowledge. These observed safety/toxicity profile advantages, as well as Karenitecin's oral bioavailability, provide additional support for the use of Karenitecin in multiple patient treatment settings, including for maintenance therapy, adjuvant and neoadjuvant therapy, and in the outpatient treatment setting.

Moreover, the mucinous adenocarcinoma sub-type of ovarian cancer represents a completely unaddressed indication where, presently, no effective treatment modality exists.

All patents, publications, scientific articles, web sites, and the like, as well as other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicant reserves the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicant reserves the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in the written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The present invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”. The letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicant reserves the right to revise the application or claims to refer specifically to any individual member or any subgroup of members of the Markush group.

Other embodiments are within the following claims. The patent may not be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. A method of treatment of a subject having advanced ovarian cancer, including the mucinous adenocarcinoma-subtype of ovarian cancer; wherein said method is comprised of the i.v. and/or oral administration of Karenitecin in an amount sufficient to provide a therapeutic benefit to the subject having advanced ovarian cancer, including the mucinous adenocarcinoma-subtype of ovarian cancer.

2. A method of treatment of a subject having advanced ovarian cancer, including the mucinous adenocarcinoma-subtype of ovarian cancer, and/or where the subject's advanced ovarian cancer is refractory or resistant to treatment with platinum and/or taxane cancer treating agents; wherein said method is comprised of the i.v. and/or oral administration of Karenitecin in an amount sufficient to provide a therapeutic benefit to the subject having advanced ovarian cancer, including the mucinous adenocarcinoma-subtype of ovarian cancer, and/or where the subject's advanced ovarian cancer is refractory or resistant to platinum and/or taxane cancer treating agents.

3. The method of claim 1 or claim 2, wherein said method consists of the administration of Karenitecin in a dosage of 1.0 mg/m²/day by a 60 minute i.v. infusion for the first 5 consecutive days of a treatment cycle that is comprised of 21 total days.

4. The method of claim 1 or claim 2, wherein the number of treatment cycles is at least 6 cycles.

5. The method of claim 1 or claim 2, wherein said method consists of the administration of Karenitecin in a dosage of 0.5 mg/m²/day in a single oral dose 3-times per week for 3 consecutive weeks, followed by a one week rest period from treatment.

6. A method for increasing the time period of Progression Free Survival (PFS) in a subject having advanced ovarian cancer, including the mucinous adenocarcinoma-subtype of ovarian cancer; wherein said method is comprised of the oral and/or i.v. administration of Karenitecin in an amount sufficient to provide an increase in the time period of Progression Free Survival (PFS) in the subject having advanced ovarian cancer, including the mucinous adenocarcinoma-subtype of ovarian cancer.

7. A method for increasing the time period of Progression Free Survival (PFS) in a subject having advanced ovarian cancer, including the mucinous adenocarcinoma-subtype of ovarian cancer, and/or where the subject's cancer is refractory or resistant to platinum and/or taxane cancer treating agents; wherein said method is comprised of the oral and/or i.v. administration of Karenitecin in an amount sufficient to provide a therapeutic benefit to the subject having advanced ovarian cancer, including the mucinous adenocarcinoma-subtype of ovarian cancer, and/or where the subject's cancer is refractory or resistant to platinum and/or taxane cancer treating agents.

8. A method for increasing the time period of Progression Free Survival (PFS) while concomitantly reducing cancer treating agent-related toxicities in a subject having advanced ovarian cancer, including the mucinous adenocarcinoma-subtype of ovarian cancer, and/or where the subject's advanced ovarian cancer is refractory or resistant to platinum and/or taxane cancer treating agents;
wherein said method is comprised of the oral and/or i.v. administration of Karenitecin in an amount sufficient to provide an increase in the time period of Progression Free Survival (PFS) while concomitantly reducing cancer treating agent-related toxicities in the subject having advanced ovarian cancer, including the mucinous adenocarcinoma-subtype of ovarian cancer, and/or where the subject's advanced ovarian cancer is refractory or resistant to platinum and/or taxane cancer treating agents.

9. The method of claim 8, wherein the cancer treating agent-related toxicities are selected from the group consisting of: hematological, gastrointestinal, neurological, and/or anorexia.

10. A method for treating a subject having advanced ovarian cancer, including the mucinous adenocarcinoma-subtype of ovarian cancer, and/or where the subject's advanced ovarian cancer is refractory or resistant to platinum and/or taxane cancer treating agents, while also concomitantly reducing the occurrence and/or grade of the occurrence of cancer treating agent-induced adverse effects to said subject;
wherein said method is comprised of the oral and/or i.v. administration of Karenitecin in an amount sufficient to provide a therapeutic benefit to the subject having advanced ovarian cancer, including the mucinous adenocarcinoma-subtype of ovarian cancer, and/or the subject's advanced ovarian cancer is refractory or resistant to platinum and/or taxane cancer treating agents, while also concomitantly reducing the occurrence and/or the grade of occurrence of cancer treating agent-induced adverse effects.

11. The method of claim 10, wherein said cancer treating agent-induced adverse effects are selected from the group consisting of: anemia, thrombocytopenia, and/or neutropenia.

12. A method for treating a subject having advanced ovarian cancer, including the mucinous subtype of ovarian cancer, and/or where the subject's advanced ovarian cancer is refractory or resistant to platinum and/or taxane cancer treating agents, while also concomitantly reducing the overall cumulative cancer treating agent-related toxicity to the subject undergoing treatment;
wherein said method is comprised of the oral and/or i.v. administration of Karenitecin in an amount sufficient to provide a therapeutic benefit to the subject having advanced ovarian cancer, including the mucinous adenocarcinoma-subtype of ovarian cancer, and/or where the subject's advanced ovarian cancer is refractory or resistant to platinum and/or taxane cancer treating agents, while also concomitantly reducing the overall cumulative cancer treating agent-related toxicity to the subject undergoing treatment.

13. A method for increasing the total number of cancer treating agent treatment cycles and/or the length of each individual cancer treating agent treatment cycle capable of being tolerated by a subject having advanced ovarian cancer, including the mucinous adenocarcinoma-subtype of ovarian cancer, and/or where the subject's advanced ovarian cancer is refractory or resistant to platinum and/or taxane cancer treating agents;
wherein said method is comprised of the oral and/or i.v. administration of Karenitecin in an amount sufficient to allow the increase of the total number of cancer treating agent treatment cycles and/or the length of each individual cancer treating agent treatment cycle capable of being tolerated by the subject having advanced ovarian cancer, including the mucinous adenocarcinoma-subtype of ovarian cancer, and/or where the subject's advanced ovarian cancer is refractory or resistant to platinum and/or taxane cancer treating agents.

14. A method for increasing the platinum-free time interval, the time that elapses after the completion of initial platinum-based therapy, in a subject having relapsed advanced ovarian cancer, including the mucinous adenocarcinoma-subtype of ovarian cancer, and/or where the subject's advanced ovarian cancer is refractory or resistant to platinum and/or taxane chemotherapeutic agents;
wherein said method is comprised of the oral and/or i.v. administration of Karenitecin in an amount sufficient to provide an increase in the platinum-free time interval by allowing the time to be extended before the subject receives another platinum-based therapy, in the subject having relapsed advanced ovarian cancer, including the mucinous adenocarcinoma-subtype of ovarian cancer, and/or where the subject's advanced ovarian cancer is refractory or resistant to platinum and/or taxane chemotherapeutic agents.

15. A method for increasing the platinum-free time interval, the time that elapses after the completion of initial platinum-based therapy, in a subject having relapsed advanced ovarian cancer which is refractory or resistant to platinum and/or taxane chemotherapeutic agents; wherein said method is comprised of the oral and/or i.v. administration of Karenitecin in an amount sufficient to provide a therapeutic benefit to the subject having relapsed advanced ovarian cancer which is refractory or resistant to platinum and/or taxane chemotherapeutic agents.

16. A method to decrease the cancer antigen 125 marker (CA-125) levels in a subject having advanced ovarian cancer, including the mucinous adenocarcinoma-subtype of ovarian cancer, and/or where the subject's advanced ovarian cancer is refractory or resistant to platinum and/or taxane chemotherapeutic agents, and/or where the subject is post-menopausal;
wherein said method is comprised of the oral and/or i.v. administration of Karenitecin in an amount sufficient to decrease the CA-125 marker levels in the subject having advanced ovarian cancer, including the mucinous adenocarcinoma-subtype of ovarian cancer, and/or where the subject's advanced ovarian cancer is refractory or resistant to platinum and/or taxane chemotherapeutic agents, and/or where the subject is post-menopausal; and
wherein the amount of CA-125 marker levels in said subject having advanced ovarian cancer, including the mucinous adenocarcinoma-subtype of ovarian cancer, is measured: (i) prior to beginning the treatment regimen with Karenitecin, and (ii) during the treatment regimen with Karenitecin, with both the time interval between CA-125 marker level measurements and the amount of Karenitecin administered to said subject being dependent upon the CA-125 marker levels which are found in said subject having advanced ovarian cancer.

17. A composition for the treatment of a subject having advanced ovarian cancer, including the mucinous adenocarcinoma-subtype of ovarian cancer, and/or where the subject's advanced ovarian cancer is refractory or resistant to platinum and/or taxane chemotherapeutic agents;
wherein said composition is comprised of Karenitecin, Germanium-substituted Karenitecin, deuterated Karenitecin, and/or “flipped” E-ring Karenitecin administered by oral and/or i.v. methodologies in an amount sufficient to provide a therapeutic benefit to the subject having advanced ovarian cancer, including the mucinous adenocarcinoma-subtype of ovarian cancer, and/or where the subject's advanced ovarian cancer is refractory or resistant to platinum and/or taxane chemotherapeutic agents.

18. A composition for the treatment of advanced ovarian cancer, including the mucinous adenocarcinoma-subtype of ovarian cancer, and/or where the subject's advanced ovarian cancer is refractory or resistant to platinum and/or taxane chemotherapeutic agents;
wherein said composition is comprised of Karenitecin and a specific protein-targeting monoclonal antibody or another cancer treating agent which is chemically-attached to or conjugated with the Karenitecin; and
wherein said composition is administered, either concomitantly or in series, via oral and/or i.v. means, in an amount sufficient to provide a therapeutic benefit to the subject having advanced ovarian cancer, including the mucinous adenocarcinoma-subtype of ovarian cancer and/or where the subject's cancer is refractory or resistant to platinum and/or taxane chemotherapeutic agents.

19. The composition of claim 18, wherein the specific protein-targeting monoclonal antibody which attached to or conjugated with the Karenitecin is selected from the group consisting of T-DM1, inotuzumar, and necitumumab.

20. A composition for the treatment of advanced ovarian cancer, including the mucinous adenocarcinoma-subtype of ovarian cancer, and/or where the advanced ovarian cancer is refractory or resistant to platinum and/or taxane chemotherapeutic agents;
wherein said composition is comprised of Karenitecin and one or more cancer treating agents administered either concomitantly or in series, via oral and/or i.v. means, in an amount sufficient to provide a therapeutic benefit to the subject having advanced ovarian cancer, including the mucinous adenocarcinoma-subtype of ovarian cancer, and/or where the subject's cancer is refractory or resistant to platinum and/or taxane chemotherapeutic agents.

21. The composition of claim 20, wherein the one or more cancer treating agents are selected from the group consisting of: fluropyrimidines; pyrimidine nucleosides; purine nucleosides; anti-folates, platinum agents; anthracyclines/anthracenediones; epipodophyllotoxins; camptothecins; vinca alkaloids; taxanes; epothilones; antimicrotubule agents; alkylating agents; antimetabolites; topoisomerase inhibitors; aziridine-containing compounds; antivirals; hormones; hormonal complexes; antihormonals; enzymes, proteins, chemoenhancing agents, chemosupportive agents, peptides and polyclonal and/or monoclonal antibodies and various other cytotoxic and cytostatic agents.

22. The composition of claim 20, wherein the cancer treating agent is disodium 2,2′-dithio-bis-ethane sulfonate (Tavocept, BNP7787, dimesna).

23. A method to mitigate or prevent the development of chemotherapeutic or biologic drug-resistance or cancer treating agent treatment-resistance in a subject having advanced ovarian cancer, including the mucinous adenocarcinoma-subtype of ovarian cancer, and/or where the subject's cancer is refractory or resistant to platinum and/or taxane chemotherapeutic agents;
wherein said method is comprised of the oral and/or i.v. administration of Karenitecin in an amount sufficient to mitigate or prevent the development of chemotherapeutic drug-resistance or biologic treatment resistance in the subject having advanced ovarian cancer, including the mucinous adenocarcinoma-subtype of ovarian cancer, and/or where the cancer is refractory or resistant to platinum and/or taxane chemotherapeutic agents.

24. A method for treating a subject having advanced ovarian cancer, including the mucinous adenocarcinoma-subtype of ovarian cancer, and/or where the advanced ovarian cancer is refractory or resistant to platinum and/or taxane chemotherapeutic agents, while also concomitantly reducing the occurrence and/or grade of the occurrence of toxicities induced by cancer treating agents, including cancer treating agent-induced toxicities and/or improving the adverse effects profile of chemotherapeutic administration;
wherein said method is comprised of the oral and/or i.v. administration of one or more camptothecin cancer treating agents in an amount sufficient to provide a therapeutic benefit to the subject having advanced ovarian cancer, including the mucinous adenocarcinoma-subtype of ovarian cancer, and/or the advanced ovarian cancer is refractory or resistant to platinum and/or taxane chemotherapeutic agents, while concomitantly reducing the occurrence and/or grade of the occurrence of cancer treatment agent-induced toxicities and/or improving the adverse effects profile of chemotherapeutic administration.

25. The method of claim 24, wherein the one or more camptothecin chemotherapeutic agents are selected from the group consisting of: Karenitecin, Germanium-substituted Karenitecin, deuterated Karenitecin, and “flipped” E-ring Karenitecin.

26. A method for the treatment of a subject having one or more cancer types selected from the group consisting of: (i) advanced solid tumors; (ii) refractory or recurrent solid tumors; (iii) recurrent malignant glioma; (iv) primary malignant glioma; (v) persistent or recurrent epithelial ovarian or primary peritoneal carcinoma; (vi) malignant melanoma; (vii) relapsed or refractory non-small cell lung cancer; (viii) neuroendocrine cancer; (ix) breast cancer; (x) pancreatic cancer; (xi) melanoma; (xii) peritoneal papillary adenocarcinoma; (xiii) leiomyosarcoma; (xiv) refractory small cell cancer; (xv) colorectal cancer; (xvi) prostate cancer; (xvii) sarcoma; and/or (xviii) carcinoma of the parotid gland;
wherein said method is comprised of the oral and/or i.v. administration of Karenitecin in an amount sufficient to provide a therapeutic benefit to the subject having one or more cancer types selected from the group consisting of: (i) advanced solid tumors; (ii) refractory or recurrent solid tumors; (iii) recurrent malignant glioma; (iv) primary malignant glioma; (v) persistent or recurrent epithelial ovarian or primary peritoneal carcinoma; (vi) malignant melanoma; (vii) relapsed or refractory non-small cell lung cancer; (viii) neuroendocrine cancer; (ix) breast cancer; (x) pancreatic cancer; (xi) melanoma; (xii) peritoneal papillary adenocarcinoma; (xiii) leiomyosarcoma; (xiv) refractory small cell cancer; (xv) colorectal cancer; (xvi) prostate cancer; (xvii) sarcoma; and/or (xviii) carcinoma of the parotid gland.

27. A method for the treatment of a subject having one or more cancer types selected from the group consisting of: (i) advanced solid tumors; (ii) refractory or recurrent solid tumors; (iii) recurrent malignant glioma; (iv) primary malignant glioma; (v) third-line treatment of persistent or recurrent epithelial ovarian or primary peritoneal carcinoma; (vi) malignant melanoma; (vii) relapsed or refractory non-small cell lung cancer; (viii) neuroendocrine cancer; (ix) breast cancer; (x) pancreatic cancer; (xi) melanoma; (xii) peritoneal papillary adenocarcinoma; (xiii) leiomyosarcoma; (xiv) refractory small cell cancer; (xv) colorectal cancer; (xvi) prostate cancer; (xvii) sarcoma; and/or (xviii) carcinoma of the parotid gland;
wherein said method is comprised of administering therapeutically-effective doses of Karenitecin and one or more cancer treating agents either concomitantly or in series, via oral and/or i.v. means, to the subject having one or more cancer types selected from the group consisting of: (i) advanced solid tumors; (ii) refractory or recurrent solid tumors; (iii) recurrent malignant glioma; (iv) primary malignant glioma; (v) third-line treatment of persistent or recurrent epithelial ovarian or primary peritoneal carcinoma; (vi) malignant melanoma; (vii) relapsed or refractory non-small cell lung cancer; (viii) neuroendocrine cancer; (ix) breast cancer; (x) pancreatic cancer; (xi) melanoma; (xii) peritoneal papillary adenocarcinoma; (xiii) leiomyosarcoma; (xiv) refractory small cell cancer; (xv) colorectal cancer; (xvi) prostate cancer; (xvii) sarcoma; and/or (xviii) carcinoma of the parotid gland.

28. The composition of claim 27, wherein the one or more cancer treating agents are selected from the group consisting of: fluropyrimidines; pyrimidine nucleosides; purine nucleosides; anti-folates, platinum agents; anthracyclines/anthracenediones; epipodophyllotoxins; camptothecins; vinca alkaloids; taxanes; epothilones; antimicrotubule agents; alkylating agents; antimetabolites; topoisomerase inhibitors; aziridine-containing compounds; antivirals; hormones; hormonal complexes; antihormonals; enzymes, proteins, chemoenhancing agents, chemosupportive agents, peptides and polyclonal and/or monoclonal antibodies, and various other cytotoxic and cytostatic agents.

29. The composition of claim 27, wherein the cancer treating agent is disodium 2,2′-dithio-bis-ethane sulfonate (Tavocept, BNP7787, dimesna).

30. A method to increase the progression-free survival (PFS) in a subject diagnosed with advanced epithelial ovarian cancer which exhibits evidence of being refractory or resistant to platinum-based and/or taxane-based cancer treating agents; wherein said method is comprised of treating said subjects with Karenitecin, rather than Topotecan.

31. A method to increase the progression-free survival (PFS) in a subject diagnosed with the mucinous adenocarcinoma subtype of ovarian cancer; wherein said method is comprised of treating said subjects with Karenitecin, rather than Topotecan; and
wherein said method further induces an increase in the overall survival (OS) of the subject, who is treated with Karenitecin, rather than Topotecan

32. A method to induce a reduction in grade 3 or 4 anemia events, and/or a reduction in grade 3 or 4 thrombocytopenia events, and/or a reduction in grade 4 neutropenia events during treatment of a subject with cancer, wherein said method is comprised of the i.v. and/or oral administration of Karenitecin in an amount sufficient to provide a therapeutic benefit to the subject.

33. A method for increasing the total number of cancer treating agent treatment cycles and/or the length of each individual cancer treating agent treatment cycle capable of being tolerated by a subject having cancer and/or where the subject's cancer is refractory or resistant to one or more cancer treating agents;
wherein said method is comprised of the oral and/or i.v. administration of Karenitecin in an amount sufficient to allow the increase of the total number of cancer treating agent treatment cycles and/or the length of each individual treatment cycle capable of being tolerated by the subject having cancer and/or where the subject's cancer is refractory or resistant to one or more cancer treating agents.

34. The method of claim 33, wherein the cancer is further selected from the group consisting of: colorectal cancer, gastric cancer, esophageal cancer, cancer of the biliary tract, gallbladder cancer, breast cancer, brain cancer and cancer of the Central Nervous System, cervical cancer, ovarian cancer, endometrial cancer, vaginal cancer, uterine cancer, prostate cancer, hepatic cancer, adenocarcinoma, pancreatic cancer, lung cancer, myeloma, lymphoma, and cancers of the blood.

35. A method to adjust the timing and dosage of Karenitecin administered to a subject having advanced ovarian cancer, including the mucinous adenocarcinoma-subtype of ovarian cancer, and/or where the subject's advanced ovarian cancer is resistant or refractory to platinum and/or taxane cancer treating agents;
wherein the adjustment of the timing and dosage of Karenitecin administration is based upon cancer antigen 125 (CA-125) marker levels in said subject having advanced ovarian cancer, including the mucinous adenocarcinoma-subtype of ovarian cancer, with the CA-125 marker levels being measured: (i) prior to beginning the treatment regimen with Karenitecin, and (ii) during the treatment regimen with Karenitecin, with both the time interval between CA-125 marker level measurements and the amount of Karenitecin administered to said subject being dependent upon the CA-125 marker levels which measured in said subject having advanced ovarian cancer, including the mucinous adenocarcinoma-subtype of ovarian cancer; and
wherein said method is comprised of the oral and/or i.v. administration of Karenitecin in an amount sufficient to decrease the CA-125 marker levels in the subject having advanced ovarian cancer, including the mucinous adenocarcinoma-subtype of ovarian cancer, and/or where the subject's advanced ovarian cancer is refractory or resistant to platinum and/or taxane cancer treating agents.

36. The method of claim 35, wherein the adjustment of the timing and dosage of Karenitecin administered is based upon the levels of the Mucin 16 marker (MUC16) levels in a subject having advanced ovarian cancer, including the mucinous adenocarcinoma-subtype of ovarian cancer, and/or where the subject's advanced ovarian cancer is refractory or resistant to platinum and/or taxane chemotherapeutic agents.

37. A method to treat cancers histologically-characterized as being of the mucinous type; wherein said method is comprised of the i.v. and/or oral administration of Karenitecin in a therapeutically-effective dose to the subject having one or more cancers which have been histologically-characterized as being of the mucinous type.

38. A method to treat a subject having advanced ovarian cancer, including the mucinous adenocarcinoma-subtype of ovarian cancer, and/or where the subject's advanced ovarian cancer is refractory or resistant to platinum and/or taxane chemotherapeutic agents, and where said subject is also suffering from cancer treating agent-associated toxicity or toxicities; and
wherein said method is comprised of the oral and/or i.v. administration of a therapeutically-effective dose of Karenitecin to the subject having advanced ovarian cancer, including the mucinous adenocarcinoma-subtype of ovarian cancer, and/or where the subject's advanced ovarian cancer is refractory or resistant to platinum and/or taxane chemotherapeutic agents, and where the subject is also suffering from cancer treating agent-associated toxicity or toxicities.

39. A method for the administration of Karenitecin on a continuous, ongoing or regular periodic basis, including by means of maintenance therapy, adjuvant therapy, outpatient treatment, and/or neoadjuvant therapy, to a subject having cancer; wherein said method is comprised of the oral and/or i.v. administration of a therapeutically-effective dose of Karenitecin to the subject in an amount sufficient to allow the administration of Karenitecin on a continuous, ongoing or regular periodic basis, including by means of maintenance therapy, adjuvant therapy, outpatient treatment, and/or neoadjuvant therapy.

40. A method for increasing Progression Free Survival (PFS) in a subject with advanced ovarian cancer; wherein the subject's advanced ovarian cancer has been classified into detailed ovarian cancer histological sub-categories selected from the group consisting of: (i) the “Histological Stage: G1-well differentiated” sub-category; (ii) the “ECOG Performance Status 2” sub-category; (iii) the “ECOG Performance Status 0” sub-category; (iv) the “Histopathology class: Adenocarcinoma (grade ≧2) not otherwise specified” sub-category; (v) the “Histopathology class: serous adenocarcinoma” sub-category; (vi) the “Histological Stage: G2-moderately differentiated” sub-category; (vii) the “FIGO Stage IV” sub-category; (viii) the “FIGO Stage IV” sub-category; (ix) the “FIGO Stage IIIB; Ovary as primary site of disease” sub-category; and (x) the “best response stable disease (SD) after 6 cycles in a first-line setting” sub-category; and
wherein said method is comprised of the oral and/or i.v. administration of a therapeutically-effective dose of Karenitecin to the subject with advanced ovarian cancer; wherein said subject's advanced ovarian cancer has been classified into detailed ovarian cancer histological sub-categories.

41. A method for increasing Overall Survival (OS) in a subject with advanced ovarian cancer; wherein the subject's advanced ovarian cancer has been classified into detailed ovarian cancer histological sub-categories selected from the group consisting of: (i) the “mucinous adenocarcinoma ovarian cancer” sub-category; (ii) the “advanced epithelial ovarian cancer” sub-category; (iii) the “FIGO Stage IV” sub-category; (iv) the “Histopathology class: Adenocarcinoma (grade ≧2) not otherwise specified” subcategory; (iv) the “Histological Stage: G1-well differentiated” sub-category; and (v) the “Histopathology Class: Undifferentiated carcinoma” sub-category; and
wherein said method is comprised of the oral and/or i.v. administration of a therapeutically-effective dose of Karenitecin to the subject with advanced ovarian cancer; wherein said subject's advance ovarian cancer has been classified into detailed ovarian cancer histological sub-categories.

42. A method to reduce or prevent cancer treating agent-induced toxicities in a subject having cancer, including where the subject's cancer is refractory or resistant to one or more cancer treating agents;
wherein said method is comprised of the oral and/or i.v. administration of a therapeutically-effective dose of Karenitecin to the subject having cancer, including where the subject's cancer is refractory or resistant to one or more cancer treating agents, and where the subject is also suffering from cancer treating agent-associated toxicity or toxicities.

43. A method to improve the Quality of Life (QOL) in a subject having cancer, and/or where the subject's cancer is refractory or resistant to one or more cancer treating agents, wherein said method is comprised of the oral and/or i.v. administration of a therapeutically-effective dose of Karenitecin to the subject in an amount sufficient to allow an improvement in the Quality of Life (QOL) in the subject having cancer and/or where the subject's cancer is refractory or resistant to one or more cancer treating agents.

44. A method to reduce or prevent cancer treating agent-induced toxicities in a subject having advanced ovarian cancer, including the mucinous adenocarcinoma-subtype of ovarian cancer, and/or where the subject's advanced ovarian cancer is refractory or resistant to platinum and/or taxane chemotherapeutic agents, and where said subject is also suffering from cancer treating agent-associated toxicity or toxicities; and
wherein said method is comprised of the oral and/or i.v. administration of a therapeutically-effective dose of Karenitecin to the subject having advanced ovarian cancer, including the mucinous adenocarcinoma-subtype of ovarian cancer, and/or where the subject's advanced ovarian cancer is refractory or resistant to platinum and/or taxane chemotherapeutic agents, and where the subject is also suffering from cancer treating agent-associated toxicity or toxicities.
